(19) United States
(12) Patent Application Publication
(10) Pub. No.: US 2011/0201629 A1
(43) Pub. Date: Aug. 18, 2011

(54) CYCLOHEXYL AMIDE DERIVATIVES AS CRF RECEPTOR ANTAGONISTS

(76) Inventors: Benjamin Atkinson, Claverton Down (GB); David Beattie, Horsham (GB); Andrew James Culshaw, Greenford (GB); James Dale, Hosrsham (GB); Nicholas James Devereux, Ash (GB); Jeffrey McKenna, Horsham (GB)

(21) Appl. No.: 13/017,399

(22) Filed: Jan. 31, 2011

Related U.S. Application Data
(60) Provisional application No. 61/300,200, filed on Feb. 1, 2010.
(60) Provisional application No. 61/424,272, filed on Dec. 17, 2010.

(51) Int. Cl. A61K 31/522; C07D 209/34; A61K 31/404; A61P 25/18; A61P 25/28; A61P 29/00; A61P 25/30; A61P 1/08; A61P 25/24; C07D 401/12; A61K 31/4439; C07D 471/04; A61K 31/437; C07D 471/10; C07D 217/24; A61K 31/472; C07D 498/04; C07D 473/28

(52) U.S. Cl. 514/263.22; 548/486; 514/418; 546/277.7; 514/339; 546/113; 514/300; 546/273.7; 514/338; 546/15; 514/278; 546/17; 546/141; 514/309; 546/116; 514/302; 544/276

(57) ABSTRACT

There are described cyclohexyl amide derivatives of Formula I,

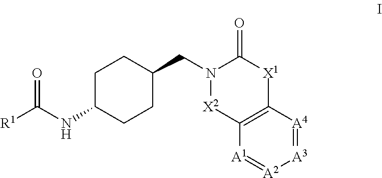

which are useful as corticotropin releasing factor (CRF) receptor antagonists and as pharmaceuticals.

### CROSS-REFERENCES TO RELATED APPLICATIONS

This application claims the benefit of U.S. Provisional Application No. 61/300,200, filed Feb. 1, 2010, and U.S. Provisional Application No. 61/424,272, filed Dec. 17, 2010; the contents of those applications are incorporated herein by reference.

### FIELD OF THE INVENTION

The present invention relates to cyclohexyl amide derivatives, their preparation, their use as pharmaceuticals and pharmaceutical compositions containing them. More particularly the present invention relates to their use as corticotropin releasing factor (CRF) receptor antagonists.

### SUMMARY OF THE INVENTION

In a first aspect of the invention we provide a compound of formula I;

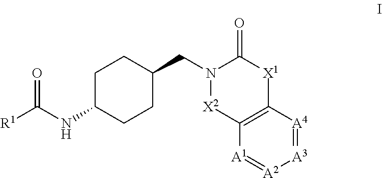

in which R¹ is phenyl or a 6-membered heteroaryl each of which may be optionally substituted by one or more substituents selected from the group alkyl C1 to 10, alkoxy C1 to 10, halogen and haloalkyl C1 to 10;

X¹ is a bond or is —CR²R³—, —NR⁴—, —O— or —CR⁵R⁶CR⁷R⁸—;

X² is a bond or is —CR⁹R¹⁰— or —CR¹¹R¹²CR¹³R¹⁴—;

provided that when X¹ is —CR⁵R⁶CR⁷R⁸— then X² is not —CR¹¹R¹²CR¹³R¹⁴— and only one of X¹ and X² may be a bond;

A¹ is —N— or CR¹⁵;

A² is CR¹⁶;

A³ is —N— or CR¹⁷;

A⁴ is —N— or CR¹⁸, provided that no more than two of A¹, A³ and A⁴ is —N—; or

R², R³, R⁵, R⁶, R⁷, R⁸, R⁹, R¹⁰, R¹¹, R¹², R¹³ and R⁶, which may be the same or different, are each hydrogen, alkyl C1 to 10 or halogen, or a pair of R² and R³, R⁵ and R⁶, R⁷ and R⁸, R⁹ and R¹⁰, R¹¹ and R¹², and R¹³ and R¹⁴, together form a 3- to 6-membered saturated carbocyclic or heterocyclic ring containing 1 or 2 heteroatoms;

R⁴ is hydrogen or alkyl C1 to 10;

R¹⁵, R¹⁶, R¹⁷ and R¹⁸, which may be the same or different, are each hydrogen, alkyl C1 to 10, alkoxy C1 to 10, halogen or haloalkoxy C1 to 10;

and isomers thereof;

in free form or in salt form.

For purposes of interpreting this specification, the following definitions will apply and whenever appropriate, terms used in the singular will also include the plural and vice versa.

As used herein, the term “alkyl” refers to a fully saturated, branched or unbranched hydrocarbon moiety, i.e. primary, secondary or tertiary alkyl or, where appropriate, cycloalkyl or alkyl substituted by cycloalkyl, they may also be saturated or unsaturated alkyl groups. Where not otherwise identified, preferably the alkyl comprises 1 to 20 carbon atoms, more preferably 1 to 16 carbon atoms, 1 to 10 carbon atoms, 1 to 7 carbon atoms, or 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl and the like.

As used herein, the term “haloalkyl” refers to an alkyl as defined herein, that is substituted by one or more halo groups as defined herein. Preferably the haloalkyl can be monohaloalkyl, dihaloalkyl or polyhaloalkyl including perhaloalkyl. A monohaloalkyl can have one iodo, bromo, chloro or fluoro within the alkyl group. Dihaloalkyl and polyhaloalkyl groups can have two or more of the same halo atoms or a combination of different halo groups within the alkyl. Preferably, the polyhaloalkyl contains up to 12, or 10, or 8, or 6, or 4, or 3, or 2 halo groups. Non-limiting examples of haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhaloalkyl refers to an alkyl having all hydrogen atoms replaced with halo atoms.

As used herein, the term “alkoxy” refers to alkyl-O—, wherein alkyl is defined herein above. Representative examples of alkoxy include, but are not limited to, methoxy, ethoxy, propoxy, 2-propoxy, butoxy, tert-butoxy, pentyloxy, hexyloxy, cyclopropyloxy-, cyclohexyloxy- and the like. Preferably, alkoxy groups have about 1-7, more preferably about 1-4 carbons.

The term “heterocyclyl” or “heterocyclic” further refers to heterocyclic groups as defined herein substituted with 1, 2 or 3 substituents selected from the groups consisting of the following:

(a) alkyl;

(b) hydroxy (or protected hydroxy);

(c) halo;

(d) haloalkyl;

(e) oxo, i.e., ═O;

(f) amino, alkylamino or dialkylamino;

(g) alkoxy;

(h) cycloalkyl;

(i) carboxyl;

(j) heterocyclooxy, wherein heterocyclooxy denotes a heterocyclic group bonded through an oxygen bridge;

(k) alkyl-O—C(O)—;

(l) mercapto;

(m) nitro;

(n) cyano;

(o) sulfamoyl or sulfonamido;

(p) aryl;

(q) alkyl-C(O)—O—;

(r) aryl-C(O)—O—;

(s) aryl-S—;

(t) aryloxy;

(u) alkyl-S—;

(v) formyl, i.e., HC(O)—;

(w) carbamoyl;

(x) aryl-alkyl-; and

(y) aryl substituted with alkyl, cycloalkyl, alkoxy, hydroxy, amino, alkyl-C(O)—NH—, alkylamino, dialkylamino or halogen.

As used herein, the term “cycloalkyl” refers to saturated or unsaturated monocyclic, bicyclic or tricyclic hydrocarbon groups of 3-12 carbon atoms, preferably 3-9, or 3-7 carbon atoms, each of which can be optionally substituted by one, or two, or three, or more substituents, such as alkyl, halo, oxo, hydroxy, alkoxy, alkyl-C(O)—, acylamino, carbamoyl, alkyl-NH—, (alkyl)₂N—, thiol, alkyl-S—, nitro, cyano, carboxy, alkyl-O—C(O)—, sulfonyl, sulfonamido, sulfamoyl, heterocyclyl and the like. Exemplary monocyclic hydrocarbon groups include, but are not limited to, cyclopropyl, cyclobutyl, cyclopentyl, cyclopentenyl, cyclohexyl and cyclohexenyl and the like. Exemplary bicyclic hydrocarbon groups include bornyl, indyl, hexahydroindyl, tetrahydronaphthyl, decahydronaphthyl, bicyclo[2.1.1]hexyl, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, bicyclo[2.2.2]octyl and the like. Exemplary tricyclic hydrocarbon groups include adamantyl and the like.

As used herein, the term “aryl” refers to an aromatic carbocyclic ring system containing 6 to 14 ring carbon atoms, which may be unsubstituted or substituted as defined.

As used herein, the term “aryloxy” refers to both an —O-aryl and an —O-heteroaryl group, wherein aryl and heteroaryl are defined herein.

As used herein, the term “heteroaryl” refers to a 5-14 membered monocyclic- or bicyclic- or polycyclic-aromatic ring system, having 1 to 8 heteroatoms selected from N, O or S. Preferably, the heteroaryl is a 5-10 or 5-7 membered ring system. Typical heteroaryl groups include 2- or 3-thienyl, 2- or 3-furyl, 2- or 3-pyrrolyl, 2-, 4-, or 5-imidazolyl, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-1,2,4-triazolyl, 4- or 5-1,2,3-triazolyl, tetrazolyl, 2-, 3-, or 4-pyridyl, 3- or 4-pyridazinyl, 3-, 4-, or 5-pyrazinyl, 2-pyrazinyl, 2-, 4-, or 5-pyrimidinyl.

The term “heteroaryl” also refers to a group in which a heteroaromatic ring is fused to one or more aryl, cycloaliphatic, or heterocyclyl rings, where the radical or point of attachment is on the heteroaromatic ring. Nonlimiting examples include but are not limited to 1-, 2-, 3-, 5-, 6-, 7-, or 8-indolizinyl, 1-, 3-, 4-, 5-, 6-, or 7-isoindolyl, 2-, 3-, 4-, 5-, 6-, or 7-indolyl, 2-, 3-, 4-, 5-, 6-, or 7-indazolyl, 2-, 4-, 5-, 6-, 7-, or 8-purinyl, 1-, 2-, 3-, 4-, 6-, 7-, 8-, or 9-quinolizinyl, 2-, 3-, 4-, 5-, 6-, 7-, or 8-quinolinyl, 1-, 3-, 4-, 5-, 6-, 7-, or 8-isoquinolinyl, 1-, 4-, 5-, 6-, 7-, or 8-phthalazinyl, 2-, 3-, 4-, 5-, or 6-naphthyridinyl, 2-, 3-, 5-, 6-, 7-, or 8-quinazolinyl, 3-, 4-, 5-, 6-, 7-, or 8-cinnolinyl, 2-, 4-, 6-, or 7-pteridinyl, 1-, 2-, 3-, 4-, 5-, 6-, 7-, or 8-4aH carbazolyl, 1-, 2-, 3-, 4-, 5-, 6-, 7-, or 8-carbazolyl, 1-, 3-, 4-, 5-, 6-, 7-, 8-, or 9-carbolinyl, 1-, 2-, 3-, 4-, 6-, 7-, 8-, 9-, or 10-phenanthridinyl, 1-, 2-, 3-, 4-, 5-, 6-, 7-, 8-, or 9-acridinyl, 1-, 2-, 4-, 5-, 6-, 7-, 8-, or 9-perimidinyl, 2-, 3-, 4-, 5-, 6-, 8-, 9-, or 10-phenathrolinyl, 1-, 2-, 3-, 4-, 6-, 7-, 8-, or 9-phenazinyl, 1-, 2-, 3-, 4-, 6-, 7-, 8-, 9-, or 10-phenothiazinyl, 1-, 2-, 3-, 4-, 6-, 7-, 8-, 9-, or 10-phenoxazinyl, 2-, 3-, 4-, 5-, 6-, or 1-, 3-, 4-, 5-, 6-, 7-, 8-, 9-, or 10-benzisoqinolinyl, 2-, 3-, 4-, or thieno[2,3-b]furanyl, 2-, 3-, 5-, 6-, 7-, 8-, 9-, 10-, or 11-7H-pyrazino[2,3-c]carbazolyl,2-, 3-, 5-, 6-, or 7-2H-furo[3,2-b]-pyranyl, 2-, 3-, 4-, 5-, 7-, or 8-5H-pyrido[2,3-d]-o-oxazinyl, 1-, 3-, or 5-1H-pyrazolo[4,3-d]-oxazolyl, 2-, 4-, or 5-1H-imidazo[4,5-d]thiazolyl, 3-, 5-, or 8-pyrazino[2,3-d]pyridazinyl, 2-, 3-, 5-, or 6-imidazo[2,1-b]thiazolyl, 1-, 3-, 6-, 7-, 8-, or 9-furo[3,4-c]cinnolinyl, 1-, 2-, 3-, 4-, 5-, 6-, 8-, 9-, 10, or 11-4H-pyrido[2,3-c]carbazolyl, 2-, 3-, 6-, or 7-imidazo[1,2-b][1,2,4]triazinyl, 7-benzo[b]thienyl, 2-, 4-, 5-, 6-, or 7-benzoxazolyl, 2-, 4-, 5-, 6-, or 7-benzimidazolyl, 2-, 4-, 4-, 5-, 6-, or 7-benzothiazolyl, 1-, 2-, 4-, 5-, 6-, 7-, 8-, or 9-benzoxapinyl, 2-, 4-, 5-, 6-, 7-, or 8-benzoxazinyl, 1-, 2-, 3-, 5-, 6-, 7-, 8-, 9-, 10-, or 11-1H-pyrrolo[1,2-b][2]benzazapinyl. Typical fused heteroaryl groups include, but are not limited to 2-, 3-, 4-, 5-, 6-, 7-, or 8-quinolinyl, 1-, 3-, 4-, 5-, 6-, 7-, or 8-isoquinolinyl, 2-, 3-, 4-, 5-, 6-, or 7-indolyl, 2-, 3-, 4-, 5-, 6-, 7-benzofuranyl, 2-, 4-, 5-, 6-, or 7-benzo[b]thienyl, 2-, 4-, 5-, 6-, or 7-benzoxazolyl, 2-, 4-, 5-, 6-, or 7-benzimidazolyl, 2-, 4-, 5-, 6-, or 7-benzothiazolyl.

A heteroaryl group may be mono-, bi-, tri-, or polycyclic, preferably mono-, bi-, or tricyclic, more preferably mono- or bicyclic.

As used herein, the term “halogen” or “halo” refers to fluoro, chloro, bromo, and iodo.

The trans arrangement of the 1,4-cyclohexyl substituents —N(R²)C(O)R¹ and

—CH₂(oxindole) is preferred.

The term alkyl includes straight chain, branched or cyclic alkyl groups. The term haloalkyl includes mono- and poly-substituted e.g. mono-, di- or tri-halo substituted alkyl groups.

Specific compounds of formula I which may be mentioned include:
trans-2-chloro-N-[4-(6-chloro-2-oxo-2,3-dihydro-indol-1-ylmethyl)-cyclohexyl]-5-trifluoromethyl-benzamide;
trans-2-chloro-N-[4-(2-oxo-2,3-dihydro-indol-1-ylmethyl)-cyclohexyl]-5-trifluoromethyl-benzamide;
trans-5-chloro-N-[4-(3,3-dimethyl-2-oxo-2,3-dihydro-indol-1-ylmethyl)-cyclohexyl]-2-methyl-icotinamide;
trans-2-chloro-N-[4-(6-chloro-3,3-difluoro-2-oxo-2,3-dihydro-indol-1-ylmethyl)-cyclohexyl]-5-trifluoromethyl-benzamide;
trans-2-chloro-N-[4-(3,3-dimethyl-2-oxo-2,3-dihydro-pyrrolo[2,3-b]pyridin-1-ylmethyl)-cyclohexyl]-5-trifluoromethyl-benzamide;
trans-5-chloro-N-[4-(3,3-dimethyl-2-oxo-2,3-dihydro-pyrrolo[2,3-b]pyridin-1-ylmethyl)-cyclohexyl]-2-methyl-nicotinamide;

### trans-2-chloro-N-[4-(2-oxo-2,3-dihydro-pyrrolo[2,3-b]pyridin-1-ylmethyl)-cyclohexyl]-5-trifluoromethyl-benzamide;

trans-2-chloro-N-{-4-((5′-fluoro-2′-oxospiro[cyclopropane-1,3′-indoline]-1′-yl)methyl)cyclohexyl}-5-(trifluoromethyl)benzamide;
trans-5-chloro-N-[-4-((5′-fluoro-2′-oxospiro[cyclopropane-1,3′-indoline]-1′-yl)methyl)cyclohexyl]-2-methyl nicotinamide;
trans-2-chloro-N-[4-(5-methoxy-3,3-dimethyl-2-oxo-2,3-dihydro-indol-1-ylmethyl)-cyclohexyl]-5-trifluoromethyl-benzamide;
trans-2-chloro-N-[4-(6-methoxy-3,3-dimethyl-2-oxo-2,3-dihydro-pyrrolo[3,2-c]pyridin-1-ylmethyl)-cyclohexyl]-5-trifluoromethyl-benzamide;
trans-2-chloro-N-[4-((R)-3-fluoro-3-methyl-2-oxo-2,3-dihydro-indol-1-ylmethyl)-cyclohexyl]-5-trifluoromethyl-benzamide;
trans-2-chloro-N-[4-((S)-3-fluoro-3-methyl-2-oxo-2,3-dihydro-indol-1-ylmethyl)-cyclohexyl]-5-trifluoro methyl-benzamide;
trans-2-chloro-N-[4-(3,3-dimethyl-2-oxo-2,3-dihydro-indol-1-ylmethyl)-cyclohexyl]-5-trifluoromethyl-benzamide;
trans-2-chloro-N-[4-(3,3-dimethyl-2-oxo-5-trifluoromethoxy-2,3-dihydro-indol-1-ylmethyl)-cyclohexyl]-5-trifluoromethyl-benzamide;
trans-5-chloro-N-[4-(5-fluoro-3,3-dimethyl-2-oxo-2,3-dihydro-indol-1-ylmethyl)-cyclohexyl]-2-methyl-nicotinamide;
trans-5-chloro-N-[4-(4-methoxy-3,3-dimethyl-2-oxo-2,3-dihydro-indol-1-ylmethyl)-cyclohexyl]-2-methyl-nicotinamide;
trans-5-chloro-N-[4-(7-chloro-3,3-dimethyl-2-oxo-2,3-dihydro-indol-1-ylmethyl)-cyclohexyl]-2-methyl-nicotinamide;
trans-5-chloro-N-[4-(3,3-difluoro-2-oxo-2,3-dihydro-indol-1-ylmethyl)-cyclohexyl]-2-methyl-nicotinamide;
trans-2-chloro-N-[4-(3,3-difluoro-2-oxo-2,3-dihydro-indol-1-ylmethyl)-cyclohexyl]-5-trifluoromethyl-benzamide;
trans-2-chloro-N-[4-(7-methoxy-3,3-dimethyl-2-oxo-2,3-dihydro-indol-1-ylmethyl)-cyclohexyl]-5-trifluoromethyl-benzamide;
trans-5-chloro-N-[4-((R)-3-fluoro-3-methyl-2-oxo-2,3-dihydro-indol-1-ylmethyl)-cyclohexyl]-2-methyl-nicotinamide;
trans-5-chloro-N-[4-((S)-3-fluoro-3-methyl-2-oxo-2,3-dihydro-indol-1-ylmethyl)-cyclohexyl]-2-methyl-nicotinamide;
trans-5-chloro-2-methyl-N-(-4-((2-oxospiro[indoline-3,4′-piperidine]-1-yl)methyl)cyclohexyl)nicotinamide;
trans-2-chloro-N-[4-(6-methoxy-3,3-dimethyl-2-oxo-2,3-dihydro-indol-1-ylmethyl)-cyclohexyl]-5-trifluoromethyl-benzamide;
trans-2-chloro-5-trifluoromethyl-N-[4-(3,3,7-trimethyl-2-oxo-2,3-dihydro-indol-1-ylmethyl)-cyclohexyl]-benzamide;
trans-2-chloro-5-trifluoromethyl-N-[4-(3,3,4-trimethyl-2-oxo-2,3-dihydro-indol-1-ylmethyl)-cyclohexyl]-benzamide;
trans-5-chloro-N-[4-(4-chloro-3,3-dimethyl-2-oxo-2,3-dihydro-indol-1-ylmethyl)-cyclohexyl]-2-methyl-nicotinamide;
trans-5-chloro-N-[4-(6-chloro-3,3-difluoro-2-oxo-2,3-dihydro-indol-1-ylmethyl)-cyclohexyl]-2-methyl-nicotinamide;
trans-5-chloro-2-methyl-N-[4-(3,3,4-trimethyl-2-oxo-2,3-dihydro-indol-1-ylmethyl)-cyclohexyl]-nicotinamide;
trans-5-chloro-N-[4-(6-methoxy-3,3-dimethyl-2-oxo-2,3-dihydro-indol-1-ylmethyl)-cyclohexyl]-2-methyl-nicotinamide;
trans-5-chloro-N-[4-(6-chloro-3,3-dimethyl-2-oxo-2,3-dihydro-indol-1-ylmethyl)-cyclohexyl]-2-methyl-nicotinamide;
trans-5-chloro-N-[4-(6-methoxy-3,3-dimethyl-2-oxo-2,3-dihydro-pyrrolo[3,2-c]pyridin-1-ylmethyl)-cyclohexyl]-2-methyl-nicotinamide;
trans-5-chloro-N-[4-(-3-fluoro-3,5-dimethyl-2-oxo-2,3-dihydro-indol-1-ylmethyl)-cyclohexyl]-2-methyl-nicotinamide;
trans-5-chloro-N-[4-(-3-fluoro-3,5-dimethyl-2-oxo-2,3-dihydro-indol-1-ylmethyl)-cyclohexyl]-2-methyl-nicotinamide;
trans-5-chloro-N-[4-(-6-chloro-3-fluoro-3-methyl-2-oxo-2,3-dihydro-indol-1-ylmethyl)-cyclohexyl]-2-methyl-nicotinamide;
trans-5-chloro-N-[4-(-6-chloro-3-fluoro-3-methyl-2-oxo-2,3-dihydro-indol-1-ylmethyl)-cyclohexyl]-2-methyl-nicotinamide;
trans-2-chloro-N-[4-(5-methoxy-1-oxo-3,4-dihydro-1H-isoquinolin-2-ylmethyl)-cyclohexyl]-5-trifluoromethyl-benzamide;
trans-2-chloro-N-[4-(3-oxo-3,4-dihydro-1H-isoquinolin-2-ylmethyl)-cyclohexyl]-5-trifluoromethyl-benzamide;
trans-5-chloro-2-methyl-N-[4-(-3,5,6-trifluoro-3-methyl-2-oxo-2,3-dihydro-indol-1-ylmethyl)-cyclohexyl]-nicotinamide;
trans-5-chloro-2-methyl-N-[4-(-3,5,6-trifluoro-3-methyl-2-oxo-2,3-dihydro-indol-1-ylmethyl)-cyclohexyl]-nicotinamide;
trans-5-chloro-2-methyl-N-[4-(2-oxo-oxazolo[4,5-b]pyridin-3-ylmethyl)-cyclohexyl]-nicotinamide;
trans-5-chloro-2-methyl-N-[4-(2-oxo-benzooxazol-3-ylmethyl)-cyclohexyl]-nicotinamide;
trans-5-chloro-N-[4-(3,6-dimethyl-2-oxo-2,3-dihydro-imidazo[4,5-b]pyridin-1-ylmethyl)-cyclohexyl]-2-methyl-nicotinamide;
trans-5-chloro-N-[4-(3-ethyl-2-oxo-2,3-dihydro-imidazo[4,5-c]pyridin-1-ylmethyl)-cyclohexyl]-2-methyl-nicotinamide;
trans-5-chloro-N-[4-(3,7-dimethyl-2-oxo-2,3-dihydro-imidazo[4,5-b]pyridin-1-ylmethyl)-cyclohexyl]-2-methyl-nicotinamide;
trans-5-chloro-N-[4-(3,5-dimethyl-2-oxo-2,3-dihydro-imidazo[4,5-b]pyridin-1-ylmethyl)-cyclohexyl]-2-methyl-nicotinamide;
trans-5-chloro-N-[4-(1,5-dimethyl-2-oxo-1,2-dihydro-imidazo[4,5-b]pyridin-3-ylmethyl)-cyclohexyl]-2-methyl-nicotinamide;
trans-5-chloro-N-[4-(3-ethyl-2-oxo-2,3-dihydro-benzoimidazol-1-ylmethyl)-cyclohexyl]-2-methyl-nicotinamide;
trans-5-chloro-N-[4-(3-isobutyl-2-oxo-2,3-dihydro-benzoimidazol-1-ylmethyl)-cyclohexyl]-2-methyl-nicotinamide;
trans-5-chloro-N-[4-(5-methoxy-3-methyl-2-oxo-2,3-dihydro-imidazo[4,5-b]pyridin-1-ylmethyl)-cyclohexyl]-2-methyl-nicotinamide;
trans-5-chloro-2-methyl-N-[4-(3,3,5-trimethyl-2-oxo-2,3-dihydro-indol-1-ylmethyl)-cyclohexyl]-nicotinamide;
trans-5-chloro-2-methyl-N-[4-(3-methyl-2-oxo-2,3-dihydro-benzoimidazol-1-ylmethyl)-cyclohexyl]-nicotinamide;
trans-5-chloro-2-methyl-N-[4-(1-methyl-2-oxo-1,2-dihydro-imidazo[4,5-b]pyridin-3-ylmethyl)-cyclohexyl]-nicotinamide;
trans-N-[4-(3,3-Dimethyl-2-oxo-2,3-dihydro-indol-1-ylmethyl)-cyclohexyl]-2-methyl-5-trifluoromethyl-nicotinamide;
trans-5-chloro-2-methyl-N-[4-(2-oxo-2,3-dihydro-benzoimidazol-1-ylmethyl)-cyclohexyl]-nicotinamide;
Enantiomer 1 of trans-5-chloro-N-[4-(3-fluoro-3,5,6-trimethyl-2-oxo-2,3-dihydro-indol-1-ylmethyl)-cyclohexyl]-2-methyl-nicotinamide;
Enantiomer 2 of trans-5-chloro-N-[4-(3-fluoro-3,5,6-trimethyl-2-oxo-2,3-dihydro-indol-1-ylmethyl)-cyclohexyl]-2-methyl-nicotinamide;
trans-5-chloro-N-[4-(7-methoxy-3,5-dimethyl-2-oxo-2,3-dihydro-imidazo[4,5-b]pyridin-1-ylmethyl)-cyclohexyl]-2-methyl-nicotinamide;
trans-5-chloro-N-[4-(3,3-dimethyl-2-oxo-2,3-dihydro-pyrrolo[3,2-b]pyridin-1-ylmethyl)-cyclohexyl]-2-methyl-nicotinamide;
trans-5-chloro-N-[4-(2-methoxy-9-methyl-8-oxo-8,9-dihydro-purin-7-ylmethyl)-cyclohexyl]-2-methyl-nicotinamide;
trans-5-chloro-N-[4-(2-chloro-9-methyl-8-oxo-8,9-dihydro-purin-7-ylmethyl)-cyclohexyl]-2-methyl-nicotinamide;
trans-2-chloro-N-[4-(5-chloro-2-oxo-2,3-dihydro-indol-1-ylmethyl)-cyclohexyl]-5-trifluoromethyl-benzamide;
trans-2-chloro-N-[4-(6-fluoro-2-oxo-2,3-dihydro-indol-1-ylmethyl)-cyclohexyl]-5-trifluoromethyl-benzamide;
trans-2-chloro-N-[4-(5-fluoro-3,3-dimethyl-2-oxo-2,3-dihydro-indol-1-ylmethyl)-cyclohexyl]-5-trifluoromethyl-benzamide;
trans-2-chloro-N-[4-(3-ethyl-2-oxo-2,3-dihydro-benzoimidazol-1-ylmethyl)-cyclohexyl]-5-trifluoromethyl-benzamide;
trans-2-chloro-N-[4-(3-methyl-2-oxo-2,3-dihydro-imidazo[4,5-b]pyridin-1-ylmethyl)-cyclohexyl]-5-trifluoromethyl-benzamide;
trans-2-chloro-N-[4-(1-methyl-2-oxo-1,2-dihydro-imidazo[4,5-b]pyridin-3-ylmethyl)-cyclohexyl]-5-trifluoromethyl-benzamide;
trans-2-chloro-N-[4-(2-oxo-2,3-dihydro-benzoimidazol-1-ylmethyl)-cyclohexyl]-5-trifluoromethyl-benzamide;
trans-2-chloro-N-[4-(3-methyl-2-oxo-2,3-dihydro-benzoimidazol-1-ylmethyl)-cyclohexyl]-5-trifluoromethyl-benzamide;
trans-4-Fluoro-N-[4-(3-methyl-2-oxo-2,3-dihydro-benzoimidazol-1-ylmethyl)-cyclohexyl]-3-trifluoromethyl-benzamide trifluoroacetate;
trans-2,5-Dichloro-N-[4-(3-methyl-2-oxo-2,3-dihydro-benzoimidazol-1-ylmethyl)-cyclohexyl]-benzamide trifluoroacetate;
trans-N-[4-(3,3-Dimethyl-2-oxo-2,3-dihydro-indol-1-ylmethyl)-cyclohexyl]-4-fluoro-3-trifluoromethyl-benzamide;
trans-2,5-Dichloro-N-[4-(3,3-dimethyl-2-oxo-2,3-dihydro-indol-1-ylmethyl)-cyclohexyl]-benzamide; and
trans-N-[4-(3,3-Dimethyl-2-oxo-2,3-dihydro-indol-1-ylmethyl)-cyclohexyl]-3-methoxy-benzamide;
and isomers thereof;
in free or in salt form.

Therefore, according to a further aspect of the invention we provide a compound of formula I as hereinbefore described as a medicament. More particularly, we provide a compound of formula I as hereinbefore described as a corticotropin releasing factor (CRF) receptor antagonist.

According to a further aspect of the invention we provide the use of a compound of formula I as hereinbefore described in the manufacture of a medicament. More particularly, we provide the use as hereinbefore described in the manufacture of a medicament for a corticotropin releasing factor (CRF) receptor antagonist.

Furthermore it has now been found that the compounds of formula I, or a salt thereof, behave as CRF receptor antagonists. Representative compounds of the invention have no significant agonist or antagonist activity at melanin concentrating hormone receptor 1 (MCH-1) or MCH-2.

Certain compounds of formula I show antagonistic activity at both the corticotropin releasing factor receptor 1 (CRF-1) and 2 (CRF-2) and are thus dual CRF-1 and CRF-2 antagonists.

The activity of a compound according to the present invention can be assessed by the following in vitro & in vivo methods.

The CRF-1 and CRF-2 receptor antagonistic activity of the agents of the invention has been determined in vitro in the following assay:

Chinese hamster ovary (CHO) cells expressing either the human or rat recombinant CRF-1 or human CRF-2α (Chen et al, Proc Natl Acad Sci USA 90, 8967-8971, 1993; Liaw et al, Endocrinology 137, 72-77, 1996) are propagated in Dulbecco's modified Eagle medium supplemented with 10% foetal calf serum, non-essential amino acids, 100 U/ml penicillin, 100 mg/l streptomycin and 1 g/l geneticin. CHO cells expressing the rat CRF-2β receptor (Wu et al, Endocrinology 148, 1675-1687, 2007) are propagated in HAM's-F12 Glutamax supplemented with 10% foetal calf serum, 100 IU/ml penicillin, 100 mg/l streptomycin, 600 μg/ml hygromycin, 10 μg/ml blasticidin and induced with 1 μg/ml of tetracyclin for 24 hours prior to experimentation. For cyclic AMP determinations the Homogeneous Time-Resolved Fluoresce (HTRF) cAMP dynamic 2 kit (Cisbio International, France) was used as per manufacturers' instructions. CHO cells, previously cryopreserved at 3×10⁶ viable cells per ml of cell recovery media (Cat no. 12648-010, Invitrogen), were thawed, centrifuged for 7 mins at 1200 rpm and resuspended in serum free media to give a concentration of 0.5×10⁶ cells per/ml. Compounds of the invention, prepared in DMSO, and subsequently diluted 50 fold in assay buffer (1× Hanks balanced salt solution, 0.2% (w/v) bovine serum albumin, 1.7 mM isobutylmethylxanthine and 10 mM Hepes, pH7.4) were then added onto the 384 well low volume black assay plate (Corning Inc, US, Cat. 3676). 2000 cells/well were then added to the assay plate further diluting the compound 2 fold and then the plate was incubated for 15 mins at room temperature. Following incubation, buffer containing a 5 times final concentration of agonist, typically r/h CRF is added to the plate and incubated for 30 min at room temperature. Finally, d2 dye labeled cAMP and cryptate labeled anti-cAMP antibody, both made in lysis buffer, are added to the plate followed by a settling period of 1 hour at room temperature. During the settling period cAMP produced by the cells competes with the d2 labelled cAMP for the anti-cAMP cryptate. The plate is read on the Pherastar (BMG, Germany). Increasing levels of endogenous cAMP produced by cells can be followed by a decrease of FRET fluorescent signal and vice versa. Values represented by a change in arbitrary fluorescence units are converted into cAMP concentrations by use of a standard curve, the reagents for which are supplied with the kit. Antagonist dose response curves (1 nM-31.6 μM) are constructed and tested in the presence of an EC₅₀ concentration of CRF relevant to the receptor (hCRF-1=3 nM, hCRF-2α=2 nM, rCRF-1=1 nM and rCRF-2β=0.1 nM). IC₅₀ values of antagonists are calculated by fitting the percent inhibition of CRF induced cAMP response by increasing concentrations of the antagonists. The fit is performed using the nonlinear logistic function of the Activitybase software package v 5.4.5.27 (IDBS, UK).

In this test, the agents of the invention show CRF1 antagonistic activity with IC50 CRF1 values of about 1 nM to 30 μM, preferably about 1 to 500 nM. Specific data are provided in the section ‘Biological data’ . . . .

Compounds of the invention are useful for the treatment of any state with increased endogenous levels of CRF (corticotropin releasing factor) or in which the HPA (hypothalamic pituitary axis) is disregulated, or of various diseases induced or facilitated by CRF.

Compounds of the invention are in particular useful for the treatment or prevention of gastrointestinal disorders including irritable bowel syndrome with or without diarrhea, inflammatory bowel diseases, post-operative ileus, reflux disease and infectious diarrhea.

Compounds of the invention are also in particular useful for the treatment or prevention of major depressive disorders including bipolar depression, unipolar depression, single or recurrent major depressive episodes with or without psychotic features, catatonic features, melancholic features, atypical features or postpartum onset, the treatment of anxiety and the treatment of panic disorders. Other mood disorders encompassed within the term major depressive disorders include fatigue syndrome and dysthymic disorder with early or late onset and with or without atypical features, neurotic depression, post traumatic stress disorders, post operative stress and social phobia; dementia of the Alzheimer's type, with early or late onset, with depressed mood; vascular dementia with depressed mood; mood disorders induced by alcohol, amphetamines, cocaine, hallucinogens, inhalants, opioids, phencyclidine, sedatives, hypnotics, anxiolytics and other substances; schizoaffective disorder of the depressed type; and adjustment disorder with depressed mood. Major depressive disorders may also result from a general medical condition including, but not limited to, myocardial infarction, diabetes, miscarriage or abortion, etc.

Compounds of the invention are also useful in the treatment or prevention of schizophrenic disorders including paranoid schizophrenia, disorganised schizophrenia, catatonic schizophrenia, undifferentiated schizophrenia, residual schizophrenia.

Compounds of the invention are also useful in the treatment or prevention of neurodegenerative diseases such as Alzheimer's disease, Parkinson's disease, Huntington's disease, senile dementia of the Alzheimer's type, and multiinfarct dementia.

Compounds of the invention are useful as analgesics. In particular they are useful in the treatment of traumatic pain such as postoperative pain; traumatic avulsion pain such as brachial plexus; chronic pain such as arthritic pain such as occurring in osteo-, rheumatoid or psoriatic arthritis; neuropathic pain such as post-herpetic neuralgia, trigeminal neuralgia, segmental or intercostal neuralgia, fibromyalgia, causalgia, peripheral neuropathy, diabetic neuropathy, chemotherapy-induced neuropathy, AIDS related neuropathy, occipital neuralgia, geniculate neuralgia, glossopharyngeal neuralgia, reflex sympathetic dystrophy, phantom limb pain; various forms of headache such as migraine, acute or chronic tension headache, temporomandibular pain, maxillary sinus pain, cluster headache; odontalgia; cancer pain; pain of visceral origin; gastrointestinal pain; nerve entrapment pain; sport's injury pain; dysmennorrhoea; menstrual pain; meningitis; arachnoiditis; musculoskeletal pain; low back pain e.g. spinal stenosis; prolapsed disc; sciatica; angina; ankylosing spondyolitis; gout; burns; scar pain; itch; and thalamic pain such as post stroke thalamic pain.

Compounds of the invention are also useful for the treatment of dysfunction of appetite and food intake and in circumstances such as anorexia, anorexia nervosa, bulimia, obesity and metabolic syndrome.

Compounds of the invention are also useful in the treatment of sleep disorders including dysomnia, insomnia, sleep apnea, narcolepsy, and circadian rhythmic disorders.

Compounds of the invention are also useful in the treatment or prevention of cognitive disorders. Cognitive disorders include dementia, amnestic disorders and cognitive disorders not otherwise specified.

Furthermore compounds of the invention are also useful as memory and/or cognition enhancers in healthy humans with no cognitive and/or memory deficit.

Compounds of the invention are also useful in the treatment of tolerance to and dependence on a number of substances. For example, they are useful in the treatment of dependence on nicotine, alcohol, caffeine, phencyclidine (phencyclidine like compounds), or in the treatment of tolerance to and dependence on opiates (e.g. cannabis, heroin, morphine) or benzodiazepines; in the treatment of cocaine, sedative ipnotic, amphetamine or amphetamine-related drugs (e.g. dextroamphetamine, methylamphetamine) addiction or a combination thereof.

Compounds of the invention are also useful as anti-inflammatory agents. In particular they are useful in the treatment of inflammation in asthma, influenza, chronic bronchitis and rheumatoid arthritis; in the treatment of inflammatory diseases of the gastrointestinal tract such as Crohn's disease, ulcerative colitis, postoperative gastric ileus (POI), inflammatory bowel disease (IBD) and non-steroidal anti-inflammatory drug induced damage; inflammatory diseases of the skin such as herpes and eczema; inflammatory diseases of the bladder such as cystitis and urge incontinence; and eye and dental inflammation.

Compounds of the invention are also useful in the treatment of fertility problems, sexual dysfunctions and pre-term birth and non-inflammatory urogenital disorders such as overactive bladder and related urinary incontinence.

Compounds of the invention are also useful in the treatment of allergic disorders, in particular allergic disorders of the skin such as urticaria, and allergic disorders of the airways such as rhinitis.

Compounds of the invention are also useful in the treatment of mast cell activation disorders such as mastocytosis.

Compounds of the invention are also useful the treatment of Cushing's syndrome induced by drugs such as steroids or cancer such as pituitary adenoma.

Compounds of the invention are also useful in the treatment of emesis, i.e. nausea, retching and vomiting. Emesis includes acute emesis, delayed emesis and anticipatory emesis. The compounds of the invention are useful in the treatment of emesis however induced. For example, emesis may be induced by drugs such as cancer chemotherapeutic agents such as alkylating agents, e.g. cyclophosphamide, carmustine, lomustine and chlorambucil; cytotoxic antibiotics, e.g. dactinomycin, doxorubicin, mitomycin-C and bleomycin; anti-metabolites, e.g. cytarabine, methotrexate and 5-fluorouracil; vinca alkaloids, e.g. etoposide, vinblastine and vincristine; and others such as cisplatin, dacarbazine, procarbazine and hydroxyurea; and combinations thereof; radiation sickness; radiation therapy, e.g. irradiation of the thorax or abdomen, such as in the treatment of cancer; poisons; toxins such as toxins caused by metabolic disorders or by infection, e.g. gastritis, or released during bacterial or viral gastrointestinal infection; pregnancy; vestibular disorders, such as motion sickness, vertigo, dizziness and Meniere's disease; post-operative sickness; gastrointestinal obstruction; reduced gastrointestinal motility; visceral pain, e.g. myocardial infarction or peritonitis; migraine; increased intercranial pressure; decreased intercranial pressure (e.g. altitude sickness); opioid analgesics, such as morphine; and gastro-oesophageal reflux disease, acid indigestion, over-indulgence of food or drink, acid stomach, sour stomach, regurgitation, heartburn, such as episodic heartburn, nocturnal heartburn, and meal-induced heartburn and dyspepsia.

Compounds of the invention are of particular use in the treatment of gastrointestinal disorders such as irritable bowel syndrome; skin disorders such as psoriasis, pruritis and sunburn; vasospastic diseases such as angina, vascular headache and Reynaud's disease; cerebral ischeamia such as cerebral vasospasm following subarachnoid haemorrhage; fibrosing and collagen diseases such as scleroderma and eosinophilic fascioliasis; disorders related to immune enhancement or suppression such as systemic lupus erythematosus and rheumatic diseases such as fibrositis; and cough.

Compounds of the invention are useful for the treatment of neurotoxic injury which follows cerebral stroke, thromboembolic stroke, hemorrhagic stroke, cerebral ischemia, cerebral vasospam, hypoglycemia, hypoxia, anoxia, perinatal asphyxia cardiac arrest.

The utility of the agents of the invention in the above indicated diseases can be confirmed in a range of standard tests. (1) The anxiolytic activity of the agents of the invention can be confirmed in the mouse elevated plus-maze [see for example Rodgers R. J., Behavioural Pharmacology 8: 477-496 (1997) where the relevance of the elevated plus-maze is discussed on p. 486; for the method, see Rodgers R. J. et al. Ethology and Psychopharmacology (Eds S J Cooper and C A Hendrie), pp 9-44 (1994), J. Wiley, Chichester]. (2) The analgesic activity of the agents of the invention can be confirmed in rat visceral hyperalgesia models following colorectal distension [see for example Schwetz I, Am J Physiology 286: G683-G691 (2004); for the method, see Ness T. J., Brain Research 450:153-169 (1988)]. (3) The anti-diarrheal activity of the agents of the invention can be confirmed in rat defecation models during stress or CRF challenge [see for example Maillot C., Gastroenterology 119:1569-1579 (2002)].

In these tests, the agents of the invention show anxiolytic-like, visceral analgesic and anti-diarrheal effects following oral administration of 0.1 to 30 mg/kg.

Furthermore, it has surprisingly been found that CRF induced intestinal barrier dysfunction in vivo can be successfully reversed using a dual CRF receptor 1 and 2 antagonist.

Hence, in a further aspect, there is provided a dual corticotropin releasing factor receptor 1 (CRF-1) and 2 (CRF-2) antagonist for use in the treatment, alleviation or prophylaxis of a condition characterized by a barrier dysfunction of mucous epithelia, epidermis or endothelia.

In another aspect, there is provided a method of treatment, alleviation or prophylaxis of a condition characterized by a barrier dysfunction of mucous epithelia, epidermis or endothelia which comprises administering to a mammal a therapeutically effective amount of a dual corticotropin releasing factor receptor 1 (CRF-1) and 2 (CRF-2) antagonist.

According to another aspect, there is provided the use of a dual corticotropin releasing factor receptor 1 (CRF-1) and 2 (CRF-2) antagonist in the manufacture of a medicament for use in the treatment, alleviation or prophylaxis of a condition characterized by a barrier dysfunction of mucous epithelia, epidermis or endothelia.

In one embodiment, the condition is characterized by a barrier dysfunction of mucous epithelia.

In one particular embodiment, the condition is characterized by a barrier dysfunction of gastrointestinal mucous epithelia. Barrier dysfunctions of gastrointestinal mucous epithelia may be induced by radiation therapy and by drugs such as non-steroidal anti-inflammatory drugs, cancer chemotherapeutic agents, cytotoxic antibiotics, anti-metabolites, vinca alkaloids and others such as cisplatin, dacarbazine, procarbazine and hydroxyurea and combinations thereof. Barrier dysfunctions of gastrointestinal mucous epithelia may also be induced by malnutrition, total parenteral nutrition, food allergens or toxins such as toxins caused by metabolic disorders or liver diseases or by infection or released during bacterial or viral infection. More particularly, conditions characterized by a barrier dysfunction of gastrointestinal mucous epithelia for which dual corticotropin releasing factor receptor 1 (CRF-1) and 2 (CRF-2) antagonists may be useful include but are not limited to inflammatory bowel disease, irritable bowel syndrome with or without diarrhea, short bowel syndrome, chronic enteropathy such as celiac disease, postoperative ileus, cystic fibrosis, reflux disease, heartburn, infectious diarrhea, intestinal neoplasms, intestinal adenocarcinomas, diabetes, sepsis, chronic heart failure and AIDS.

In one particular embodiment, the condition is characterized by a barrier dysfunction of respiratory mucous epithelia. Barrier dysfunctions of respiratory mucous epithelia may be induced by allergens, or toxins such as toxins caused by infection or released during bacterial or viral infection. More particularly, conditions characterized by a barrier dysfunction of respiratory mucous epithelia for which dual corticotropin releasing factor receptor 1 (CRF-1) and 2 (CRF-2) antagonists may be useful include but are not limited to asthma, chronic bronchitis, rhinitis, rhinosinusitis, chronic obstructive pulmonary disease, cystic fibrosis, pneumonia, sepsis, chronic heart failure and AIDS.

In one embodiment, the condition is characterized by a barrier dysfunction of the epidermis. Barrier dysfunctions of epidermis may be induced by allergens, or toxins such as toxins caused by infection or released during bacterial or viral infection. More particularly, conditions characterized by a barrier dysfunction of epidermis for which dual corticotropin releasing factor receptor 1 (CRF-1) and 2 (CRF-2) antagonists may be useful include but are not limited to dermatitis, ichthyosis, and psoriasis.

In one embodiment, the condition is characterized by a barrier dysfunction of endothelia. Barrier dysfunctions of endothelia may be induced by allergens or toxins such as toxins caused by metabolic disorders or liver diseases or by infection or released during bacterial or viral infection. More particularly, conditions characterized by a barrier dysfunction of endothelia for which dual corticotropin releasing factor receptor 1 (CRF-1) and 2 (CRF-2) antagonists may be useful include but are not limited to ischemic injury, hypoxia, diabetes, sepsis, chronic heart failure, edema, acute lung injury, acute respiratory distress syndrome, thrombosis and cancer.

In one particular embodiment, the condition is characterized by a barrier dysfunction of the brain-blood barrier. More particularly, conditions characterized by a barrier dysfunction of the brain-blood barrier for which dual corticotropin releasing factor receptor 1 (CRF-1) and 2 (CRF-2) antagonists may be useful include but are not limited to ischemic stroke, migraine, multiple sclerosis, Alzheimer's disease, epilepsy, cancer brain metastases and encephalopathy.

Conditions characterized by a barrier dysfunction of mucous epithelia, epidermis or endothelia for which dual corticotropin releasing factor receptor 1 (CRF-1) and 2 (CRF-2) antagonists may be useful include but are not limited to inflammatory bowel disease, irritable bowel syndrome, short bowel syndrome, postoperative ileus, allergy, dermatitis, sepsis, ischemic injury, multiple sclerosis and encephalopathy (Elias and Schmuth, Curr Opin Allergy Clin Immunol 9, 437-446, 2009; Lindsberg et al., J Cerebral Blood Flow & Metabolism 30, 689-702. 2010; Marchiando et al., Annu Rev Pathol Mech Dis 5, 119-144, 2010; Öhman and Simrén, Nat Rev Gastroenterol Hepatol 7, 163-173, 2010).

For the above-mentioned indications, the appropriate dosage will of course vary depending upon, for example, the compound employed, the host, the mode of administration and the nature and severity of the condition being treated. However, in general, satisfactory results in animals are indicated to be obtained at a daily dosage of from about 0.1 to about 100 mg/kg, preferably from about 1 to about 30 mg/kg animal body weight. In larger mammals, for example humans, an indicated daily dosage is in the range from about 1 to about 500 mg, preferably from about 1 to about 100 mg of an agent of the invention, conveniently administered, for example, in divided doses up to three times a day or in sustained release form.

The agents of the invention may be administered by any conventional route, in particular enterally, preferably orally, for example in the form of tablets or capsules, or parenterally, for example in the form of injectable solutions or suspensions.

In accordance with the foregoing, the present invention also provides an agent of the invention, for use as a pharmaceutical, e.g. for the treatment of diseases induced or facilitated by CRF, such as these indicated above.

Therefore, according to a further aspect of the invention we provide a compound of formula I, or a salt thereof, for the treatment or alleviation of treatment of any state with increased endogenous level of CRF or in which the HPA (hypothalamic pituitary axis) is disregulated, or of various diseases induced or facilitated by CRF.

The agents of the invention can be administered in vivo either alone or in combination with other pharmaceutical agents, e.g. agents effective in the treatment of diseases and conditions in which an increased endogenous level of CRF plays a role or is implicated. A suitable combination consists of a compound of the present invention with one or more compounds selected from the group consisting of dopamine D2 receptor antagonists, serotonin 5-HT4 receptor agonists, serotonin 5-HT3 receptor agonists, serotonin 5-HT3 receptor antagonists, CCK1 receptor antagonists, motilin receptor agonists, g-opioid receptor antagonists, opioid receptor agonists and opiates, other CRF receptor antagonists, glutamate receptor antagonists, neurokinin receptor antagonists, histamine H2 receptor antagonists, histamine H4 receptor antagonists, proton pump inhibitors, chloride channel activators, guanylate cyclase-c activators, muscarinic receptor antagonists, antispasmodics, stimulant laxatives, osmotic laxatives, faecal softeners, absorbents and fibre supplements, antacids, GI relaxants, bismuth compounds, vanilloid receptor antagonists, anticonvulsants, NSAIDS, COX-2 inhibitors, GABAb receptor modulators, CB receptor ligands, calcium channel blockers, sodium channel blockers, tricyclic antidepressants, serotonin and noradrenaline re-uptake inhibitors, benzodiazepines, alpha-2 receptor agonists and ghrelin receptor agonists.

More specifically, a compound of the present invention may be administered as a combination with one or more compounds selected from the group consisting of dopamine D2 receptor antagonists, such as, chlorpromazine, prochlorperazine, haloperidol, alizapride, domperidone, metoclopramide and itopride; serotonin 5-HT4 receptor agonists, such as, cisapride, cinitapride, mosapride, renzapride, prucalopride, tegaserod, velusetrag, ATI-7505 and compounds described in WO 2005068461, US 2005228014, WO 2005080389, US 2006100426, US 2006100236, US 2006135764, US 2005277671, WO 2005092882, WO 2005073222, JP 2005104896, JP 2005082508, WO 2005021539, JP 2004277319, JP 2004277318, WO 2004026869, EP 1362857, WO 2006108127, US 20060183901, WO 2006127815, US 20060276482, WO 2007005951, WO 2007010390, WO 2007005951, WO 2007048643, WO 2007096352, WO 2007068739 and WO 20070117796; serotonin 5-HT3 receptor agonists, such as, pumesotrag and compounds described in WO 2007004041; serotonin 5-HT3 receptor antagonists, such as, alosetron, cilansetron, ramosetron, azasetron, ondansetron, granisetron, tropisetron, DDP225 and compounds described in WO 2006183769, WO 2006105117 and WO 2007004041; CCK1 receptor antagonists, such as, JNJ-17156516, devazepide, loxiglumide and dexloxiglumide; motilin receptor agonists, such as, motilin, atilmotin, erythromycin, alemcinal, mitemcinal, KOS-2187, 1-[4-(3-fluoro-phenylamino)-piperidin-1-yl]-2-[4-((S)-3-methyl-piperazin-1-ylmethyl)-phenyl]-ethanone and compounds described in WO 2005060693, WO 2006127252, WO 2007007018, WO 2007012479 and WO 2008000729; m-opioid receptor antagonists, such as, naxolone, alvimopan, methylnaltrexone and compounds described in US 20050203123, US 2006063792, WO 2007050802, US 2007103187, WO 2009029252, WO 2009029256, WO 2009029257 and WO 2009029253; opioid receptor agonists and opiates, such as, morphine, buprenorphine, diamorphine, dihydrocodeine, fentanyl, pethidine, asimadoline, loperamide and codeine; CRF receptor antagonists, such as, GSK876008, pexacerfont and compounds described in WO 2004069257, WO 9940089, U.S. Pat. No. 6,844,351, WO 2005013997, WO 2005014557, WO 2005023806, WO 2005026126, WO 2005028480, WO 005044793, WO 2005051954, WO 2005051954, WO 2005115399, WO 2005028480, WO 2005023806, WO 2006044958, WO 2006044821 and US 20060211710; glutamate receptor antagonists, such as, AZD9272, AZD2066, AFQ056, ADX-48621 and compounds described in WO 9902497, WO 2000020001, WO 200304758 and WO 2005030723, WO 2005077345, US 2006009443, EP 1716152, WO 2005080397, US 2006019997, WO 2005066155, WO 2005082884, WO 2005044266, WO 2005077373, EP 1713791, EP 1720860, WO 2005080379, EP 1716130, US 2006235024, WO 2005080363 WO 2006114264, WO 2006114260, WO 2006089700, WO 2006114262, WO 2006123257, US 2005272779, WO 2006048771, WO 2006123249, US 2006009477, WO 2006014185, EP 1723144, US 2006025414, US 2006004021, US 2006160857, WO 2006074884, WO 2006129199, WO 2006123244, WO 2006123255, WO 2007040982, WO 2007023290, WO 2007023242, WO 2007050050, WO 2007039781, WO 2007039782 and WO 2007023245; neurokinin receptor antagonists, such as, taletant, osanetant, casopitant, nepadutrent, saredutant, DNK-333, SLV-317, SLV321, SLV317 and compounds described in EP 96-810237, WO 2006137790, WO 2006137791, WO 2006094934, WO 2007037742 and WO 2007037743; histamine H2 receptor antagonists, such as, famotidine, cimetidine, ranitidine and nizatidine; histamine H4 receptor antagonists, such as, JNJ7777120, JNJ10191584 and compounds described in US 2006111416, WO 2006050965, WO 2005092066, WO 2005054239 US 2005070550, US 2005070527, EP 1505064, WO 2007090852, WO 2007090853, WO 2007090854, US 20070232616, US 20070238771, WO 2007117399, WO 2007031529 and WO2007072163; proton pump inhibitors, such as, omeprazole, lansoprazole, rabeprazole, tentoprazole, pantoprazole, esomeprazole, revaprazan, soraprazan and AGN201904; chloride channel activators, such as, lubiprostone; guanylate cyclase-2c activators, such as, linaclotide, guanilib, guanylin, uroguanylin and compounds described in WO 2005087797, WO 2005016244, WO 2007022531, WO 2007101158, WO 2007101161 and U.S. Pat. No. 7,041,786; muscarinic receptor antagonists, such as, darifenacin, solifenacin, atropine, dicycloverine, hycosine butyl bromide, propantheline, oxybutinin, cimetropium bromide and pinaverium bromide; antispasmodics, such as, mebeverine, octylonium bromide, trimebutine, tiropramide, alverine and peppermint oil; stimulant laxatives, such as, bisacodyl; osmotic laxatives, such as, activated charcoal with sorbitol, lactulose, magnesium hydroxide and phosphate buffered saline; faecal softeners, such as, senna concentrate, liquid paraffin and arachis oil; absorbents and fibre supplements; bulk fibre laxatives such as bran, methylcellulose, ispaghula husk and sterculia; antacids, such as, aluminium, magnesium and calcium antacids, simeticone and alginate containing preparations; GI relaxants, such as, cholestyramine resin; bismuth compounds, such as, bismuth subsalicylate; vanilloid receptor antagonists, such as, SB-705498, ABT-102, AZD1386, GRC-6211, MK-2295 and compounds described in WO 2002076946, WO 2004033435, WO 2005121116, WO 2005120510, WO 2006006740, WO 2006006741, WO 2006010445, WO 2006016218, US 2006058308, WO 2006033620, WO 2006038871, US 2006084640, US 2006089360, WO 2006058338, WO 2006063178, US 2006128689, WO 2006062981, WO 2006065646, WO 2006068618, WO 2006068592, WO 2006068593, WO 2006076646, US 2006160872, WO 200608082, US 2006183745, WO 2006095263, WO 2006102645, WO 2006100520, US 2006241296, WO 2006122200, WO 2006120481, WO 2006122250, DE 102005044814, WO 2006122772, WO 2006122777, WO 2006124753, WO 2006122799, WO 2006122770, WO 2006122769, WO 2006136245, WO 2007030761, US 20070088072, US 20070088073, US 20070105920, WO 2007042906, WO 2007045462, WO 2007050732; anticonvulsants, such as, carbemazepine, oxcarbemazepine, lamotrigine, gabapentin and pregabalin; NSAIDS, such as, aspirin, acetometaphen, ibuprofen, diclofenac, naproxen, flurbiprofen, indomethacin, piroxicam, ketoprofen, sulindac and diflunisal; COX-2 inhibitors, such as, celecoxib, rofecoxib, lumiracoxib, valdecoxib, etoricoxib and compounds described in WO 2004048314; GABAb receptor modulators, such as, racemic and (R)-baclofen, AZD3355, XP19986 and compounds described in WO 2006001750 and WO 2004000856; CB receptor ligands, such as, dronabinol, nabilone, cannabidiol, rimonabant and compounds described in WO 2002042248 and WO 2003066603; calcium channel blockers, such as, ziconotide, AGI0-003, PD-217014 and compounds described in WO 2006038594, WO 2006030211 and WO 2005068448; sodium channel blockers, such as, lamotrigine and compounds described in WO 2006023757, WO 2005097136, JP 2005206590 and WO 2005047270; tricyclic antidepressants, such as, clomipramine, amoxapine, nortripyline, amitriptyline, imipramine, desipramine, doxepin, trimipramine and protripyline; serotonin and noradrenaline re-uptake inhibitors, such as, milnacipran, desvenlafaxine, sibutramine, duloxetine, fluoxetine, paroxetine, citalopram, sertraline and fluvoxamine; benzodiazepines, such as, levotofisopam, diazepam, lorazepam, clonazepam and alprazolam; alpha-2 receptor agonists, such as, clonidine, tizanidine and guanfacine; ghrelin receptor agonists, such as, ghrelin, ibutamoren, capromorelin, tabimorelin, ipamorelin, 2-Methylalanyl-N-[1(R)-formamido-2-(1H-indol-3-yl)ethyl]-D-tryptophanamide, TZP-101, TZP-102, LY-444711, EX-1314 and compounds described in U.S. Pat. No. 6,525,203, US 20050154043, WO 2005097788, WO2006036932, WO 2006135860, US 20060079562, WO 2006010629, WO 2006009674, WO 2006009645, US 20070021331, WO 2007020013, US 20070037857, WO 2007014258, WO 2007113202, WO 2007118852, US 20080194672, US 20080051383 and US 20080051383; corticosteroids, such as, hydrocortisone, cortisone, dexamethasone, betamethasone, beclomethasone, prednisolone, 6-methylprednisolone, budesonide, mometasone furoate, ciclesonide, fluticasone propionate and fluticasone furoate; aminosalicylates, such as, mesalazine, ipsalazide, olsalazine and balsalazide; immunomodulators, such as, azathioprine, 6-mercaptopurine, methotrexate, mycophenolate mofetil, ciclosporin and tacrolimus; PDE4 inhibitors, such as, tetomilast, cilomilast, roflumilast and arofylline; antibiotics, such as, metronidazole, ornidazole and ciprofloxacin; anti-adhesion molecule agents, such as, natalizumab and MLN02; anti IL-2 agents, such as, daclizumab and basilixumab; anti CD-3 agents, such as, visilizumab; and anti-TNF agents, such as, infliximab, adalimumab, fontolizumab and certolizumab pegol; psychiatric medications comprising compounds selected from the group consisting of agomelatine, azapirones, alprazolam, amitriptyline, aniracetam, acetyl-L-carnitine, aripiprazol, acetophenazine, benzodiazepines, barbiturate, buspirone, bupropione, chlordiazepoxide, chlorazepate, clonazepam, chlorpromazine, clozapine, CX614, CX516, chlorprothixene, diphenhydramine hydroxyzine, demoxepam, diazepam, droperidol, duloxetine, donezepil, doxepine, desipramine, flurazepam, fluphenazine, fluoxetine, flupentixol, gabapentin, melatonin, ginkgo-derived compounds, galantamine, haloperidol, Hydergine (ergoloid mesylates), huperzine, isocarboxazid, imipramine, lorazepam, loxapine, meprobamate, medazepam, moclobemide, molindone, maprotiline, modafinil, memantine, methylphenicate, mesoridazine, methotrimeprazine, nortriptyline, naproxen, oxazepam, oxiracetam, olanzapine, prazepam, paroxetine, phenelzine, pipotiazine, perphenazine, promazine, pimozide, PDE4 inhibitors, quazepam, quetiapine, reboxetine, rivastigmine, prochlorperazine, risperidone, sertraline, sertindole, temazepam, triazolam, tranylcypromine, tomoxetine, thiotixene, trifluoperazine, thioridazine, zolpidem and ziprasidone.

Preferably, when X¹ is a bond X² is —CR¹¹R¹²CR¹³R¹⁴—.

Preferably, when X¹ is —CR⁵R⁶CR⁷R⁸—X² is —CR¹¹R¹²CR¹³R¹⁴—.

Preferably, when X¹ is —CR²R³X² is a bond or is —CR⁹R¹⁰—.

Preferably, when X¹ is —NR⁴— X² is a bond.

Preferably, when X¹ is —O—X² is a bond.

A group of compounds which may be mentioned are compounds of formula II;

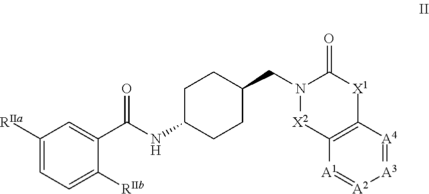

in which R^{IIa} and R^{IIb}, which may be the same or different, are each alkyl C1 to 10, halo or haloalkyl C1 to 10;

X¹, X², A¹, A², A³ and A⁴ are each as hereinbefore described;

and isomers thereof;

in free form or in salt form.

A group of compounds which may be mentioned are compounds of formula III;

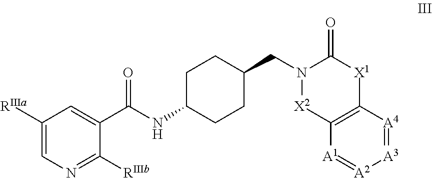

in which R^{IIIa} and R^{IIIb}, which may be the same or different, are each alkyl C1 to 10, halo or haloalkyl C1 to 10;

X¹, X², A¹, A², A³ and A⁴ are each as hereinbefore described;

and isomers thereof;

in free form or in salt form.

A group of compounds which may be mentioned are compounds of formula IV;

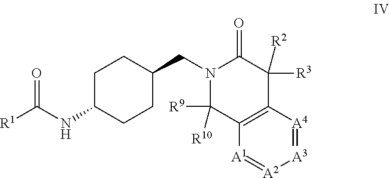

in which R¹, R², R³, R⁹, R¹⁰, A¹, A², A³ and A⁴ are each as hereinbefore described;

and isomers thereof;

in free form or in salt form.

A group of compounds which may be mentioned are compounds of formula V;

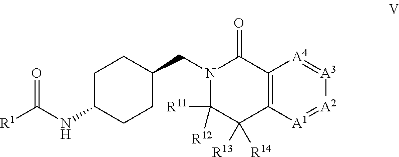

in which R¹, R², R³, R⁹, R¹⁰, A¹, A², A³ and A⁴ are each as hereinbefore described;

and isomers thereof;

in free form or in salt form.

A group of compounds which may be mentioned are compounds of formula VI;

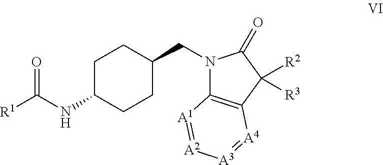

in which R¹, R², R³, A¹, A², A³ and A⁴ are each as hereinbefore described;

and isomers thereof;

in free form or in salt form.

Acid addition salts may be produced from the free bases in known manner, and vice-versa. A pharmaceutically acceptable salt is any salt of the parent compound that is suitable for administration to an animal or human. A pharmaceutically acceptable salt also refers to any salt which may form in vivo as a result of administration of an acid, another salt, or a prodrug which is converted into an acid or salt. A salt comprises one or more ionic forms of the compound, such as a conjugate acid or base, associated with one or more corresponding counter-ions. Salts can form from or incorporate one or more deprotonated acidic groups (e.g. carboxylic acids) one or more protonated basic groups (e.g. amines), or both (e.g. zwitterions).

As used herein, the term “pharmaceutically acceptable salts” refers to salts that retain the biological effectiveness and properties of the compounds of this invention and, which are not biologically or otherwise undesirable. In many cases, the compounds of the present invention are capable of forming acid and/or base salts by virtue of the presence of amino and/or carboxyl groups or groups similar thereto. Pharmaceutically acceptable acid addition salts can be formed with inorganic acids and organic acids, e.g., acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. The pharmaceutically acceptable salts of the present invention can be synthesized from a parent compound, a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

A prodrug is a compound which is converted to a therapeutically active compound after administration. For example, conversion may occur by hydrolysis of an ester group or some other biologically labile group. Prodrug preparation is well known in the art. For example “Prodrugs and Drug Delivery Systems,” which is a chapter in Richard B. Silverman, Organic Chemistry of Drug Design and Drug Action, 2d Ed., Elsevier Academic Press: Amsterdam, 2004, pp. 496-557, provides further detail on the subject.

As used herein, the term “isomers” refers to different compounds that have the same molecular formula but differ in arrangement and configuration of the atoms. Also as used herein, the term “an optical isomer” or “a stereoisomer” refers to any of the various stereo isomeric configurations which may exist for a given compound of the present invention and includes geometric isomers. It is understood that a substituent may be attached at a chiral center of a carbon, sulfur or phosphorus atom. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R—S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and may thus give rise to enantiomers, diastereomers, and other stereoisomeric forms that may be defined, in terms of absolute stereochemistry, as (R)- or (S)-. The present invention is meant to include all such possible isomers, including racemic mixtures, optically pure forms and intermediate mixtures. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

Compounds of formula (I) in optically pure form, where appropriate, can be obtained from the corresponding racemates according to well-known procedures, e.g., HPLC with chiral matrix. Alternatively, optically pure starting materials can be used.

Stereoisomeric mixtures, e.g., mixtures of diastereomers, can be separated into their corresponding isomers in a manner known per se by means of suitable separation methods. Diastereomeric mixtures, e.g., may be separated into their individual diastereomers by means of fractionated crystallisation, chromatography, solvent distribution and similar procedures. This separation may take place either at the level of a starting compound or in a compound of formula (I) itself. Enantiomers may be separated through the formation of diastereomeric salts, e.g., by salt formation with an enantiomer-pure chiral acid, or by means of chromatography, e.g., by HPLC, using chromatographic substrates with chiral ligands.

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated bonds may, if possible, be present in cis-(Z)- or trans-(E)-form.

Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

According to a further aspect of the invention we provide a method of treatment or alleviation of any state with increased endogenous level of CRF or in which the HPA (hypothalamic pituitary axis) is disregulated, or of various diseases induced or facilitated by CRF which comprises administering to a mammal a therapeutically effective amount of a compound of formula I as hereinbefore described, or a salt thereof.

We further provide a pharmaceutical composition comprising a compound of formula I as hereinbefore described, in free form or in pharmaceutically acceptable salt form, in association with a pharmaceutically acceptable adjuvant, diluent or carrier.

The pharmaceutical compositions for separate administration of the combination partners and for the administration in a fixed combination, i.e., a single galenical composition comprising at least two combination partners, according to the invention can be prepared in a manner known per se and are those suitable for enteral, such as oral or rectal, and parenteral administration to mammals, including man, comprising a therapeutically effective amount of at least one pharmacologically active combination partner alone or in combination with one or more pharmaceutically acceptable carriers, especially suitable for enteral or parenteral application.

Pharmaceutical compositions contain, e.g., from about 0.1% to about 99.9%, preferably from about 20% to about 60%, of the active ingredients. Pharmaceutical preparations for the combination therapy for enteral or parenteral administration are, e.g., those in unit dosage form, such as tablets including sugar-coated tablets, capsules, suppositories and ampoules. These are prepared in a manner known, per se, e.g., by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

The pharmaceutical composition can be formulated for particular routes of administration such as oral administration, parenteral administration, and rectal administration, etc. In addition, the pharmaceutical compositions of the present invention can be made up in a solid form including capsules, tablets, pills, granules, powders or suppositories, or in a liquid form including solutions, suspensions or emulsions. The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers etc.

Typically, the pharmaceutical compositions are tablets and gelatin capsules comprising the active ingredient together with

a) diluents, e.g., lactose, dextrose, sucrose, mannitol, sorbitol, cellulose and/or glycine;

b) lubricants, e.g., silica, talcum, stearic acid, its magnesium or calcium salt and/or polyethyleneglycol; for tablets also

c) binders, e.g., magnesium aluminum silicate, starch paste, gelatin, tragacanth, methylcellulose, sodium carboxymethylcellulose and/or polyvinylpyrrolidone; if desired

d) disintegrants, e.g., starches, agar, alginic acid or its sodium salt, or effervescent mixtures; and/or

e) absorbents, colorants, flavors and sweeteners.

Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the invention with carrier. Carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They are conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

The pharmaceutical composition or combination of the present invention can be in unit dosage of about 1-1000 mg of active ingredient(s) for a subject of about 50-70 kg, or about 1-500 mg or about 1-250 mg or about 1-150 mg or about 0.5-100 mg, or about 1-50 mg of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., preferably aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration, between about 0.1-500 mg/kg, or between about 1-100 mg/kg.

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18^{th} Ed. Mack Printing Company, 1990, pp. 1289-1329). Except insofar as any conventional carrier is incompatible with the active ingredient, its use in the therapeutic or pharmaceutical compositions is contemplated.

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to (1) at least partially alleviating, inhibiting, preventing and/or ameliorating a condition, or a disorder or a disease (i) mediated by CRF, or (ii) associated with CRF activity, or (iii) characterized by abnormal activity of CRF; or (2) reducing or inhibiting the activity of CRF; or (3) reducing or inhibiting the expression of CRF. In another non-limiting embodiment, the term “a therapeutically effective amount” refers to the amount of the compound of the present invention that, when administered to a cell, or a tissue, or a non-cellular biological material, or a medium, is effective to at least partially reducing or inhibiting the activity of CRF; or at least partially reducing or inhibiting the expression of CRF. The meaning of the term “a therapeutically effective amount” as illustrated in the above embodiment for CRF also applies by the same means to any other relevant proteins/peptides/enzymes.

As used herein, the term “subject” refers to an animal. Preferably, the animal is a mammal. A subject also refers to for example, primates (e.g., humans), cows, sheep, goats, horses, dogs, cats, rabbits, rats, mice, fish, birds and the like. In a preferred embodiment, the subject is a human.

As used herein, the term “inhibition” or “inhibiting” refers to the reduction or suppression of a given condition, symptom, or disorder, or disease, or a significant decrease in the baseline activity of a biological activity or process.

As used herein, the term “treating” or “treatment” of any disease or disorder refers in one embodiment, to ameliorating the disease or disorder (i.e., slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder.

As used herein, the term “a,” “an,” “the” and similar terms used in the context of the present invention (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

Compounds of the present invention are either obtained in the free form, as a salt thereof, or as prodrug derivatives thereof.

When both a basic group and an acid group are present in the same molecule, the compounds of the present invention may also form internal salts, e.g., zwitterionic molecules.

The present invention also provides pro-drugs of the compounds of the present invention that converts in vivo to the compounds of the present invention. A pro-drug is an active or inactive compound that is modified chemically through in vivo physiological action, such as hydrolysis, metabolism and the like, into a compound of this invention following administration of the prodrug to a subject. The suitability and techniques involved in making and using pro-drugs are well known by those skilled in the art. Prodrugs can be conceptually divided into two non-exclusive categories, bioprecursor prodrugs and carrier prodrugs. See The Practice of Medicinal Chemistry, Ch. 31-32 (Ed. Wermuth, Academic Press, San Diego, Calif., 2001). Generally, bioprecursor prodrugs are compounds, which are inactive or have low activity compared to the corresponding active drug compound, that contain one or more protective groups and are converted to an active form by metabolism or solvolysis. Both the active drug form and any released metabolic products should have acceptably low toxicity.

Carrier prodrugs are drug compounds that contain a transport moiety, e.g., that improve uptake and/or localized delivery to a site(s) of action. Desirably for such a carrier prodrug, the linkage between the drug moiety and the transport moiety is a covalent bond, the prodrug is inactive or less active than the drug compound, and any released transport moiety is acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g., stability, water solubility, suppression of an undesirable organoleptic or physiochemical property). For example, lipophilicity can be increased by esterification of (a) hydroxyl groups with lipophilic carboxylic acids (e.g., a carboxylic acid having at least one lipophilic moiety), or (b) carboxylic acid groups with lipophilic alcohols (e.g., an alcohol having at least one lipophilic moiety, for example aliphatic alcohols).

Exemplary prodrugs are, e.g., esters of free carboxylic acids and S-acyl derivatives of thiols and O-acyl derivatives of alcohols or phenols, wherein acyl has a meaning as defined herein. Preferred are pharmaceutically acceptable ester derivatives convertible by solvolysis under physiological conditions to the parent carboxylic acid, e.g., lower alkyl esters, cycloalkyl esters, lower alkenyl esters, benzyl esters, mono- or di-substituted lower alkyl esters, such as the α-(amino, mono- or di-lower alkylamino, carboxy, lower alkoxycarbonyl)-lower alkyl esters, the α-(lower alkanoyloxy, lower alkoxycarbonyl or di-lower alkylaminocarbonyl)-lower alkyl esters, such as the pivaloyloxymethyl ester and the like conventionally used in the art. In addition, amines have been masked as arylcarbonyloxymethyl substituted derivatives which are cleaved by esterases in vivo releasing the free drug and formaldehyde (Bundgaard, J. Med. Chem. 2503 (1989)). Moreover, drugs containing an acidic NH group, such as imidazole, imide, indole and the like, have been masked with N-acyloxymethyl groups (Bundgaard, Design of Prodrugs, Elsevier (1985)). Hydroxy groups have been masked as esters and ethers. EP 039,051 (Sloan and Little) discloses Mannich-base hydroxamic acid prodrugs, their preparation and use.

Furthermore, the compounds of the present invention, including their salts, can also be obtained in the form of their hydrates, or include other solvents used for their crystallization.

The present invention includes all pharmaceutically acceptable isotopically-labeled compounds of the invention, i.e. compounds of formula (I), wherein (1) one or more atoms are replaced by atoms having the same atomic number, but an atomic mass or mass number different from the atomic mass or mass number usually found in nature, and/or (2) the isotopic ratio of one or more atoms is different from the naturally occurring ratio.

Examples of isotopes suitable for inclusion in the compounds of the invention comprises isotopes of hydrogen, such as ²H and ³H, carbon, such as ¹¹C, ¹³C and ¹⁴C, chlorine, such as ³⁶Cl, fluorine, such as ¹⁸ iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labeled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances and deuterium analogues are included within the scope of the compounds of the present invention.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

Compounds of the invention, i.e. compounds of formula I that contain groups capable of acting as donors and/or acceptors for hydrogen bonds may be capable of forming co-crystals with suitable co-crystal formers. These co-crystals may be prepared from compounds of formula I by known co-crystal forming procedures. Such procedures include grinding, heating, co-subliming, co-melting, or contacting in solution compounds of formula I with the co-crystal former under crystallization conditions and isolating co-crystals thereby formed. Suitable co-crystal formers include those described in WO 2004/078163. Hence the invention further provides co-crystals comprising a compound of formula I.

The invention further includes any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure antipodes.

Compounds of the invention and intermediates can also be converted into each other according to methods generally known per se.

Within the scope of this text, only a readily removable group that is not a constituent of the particular desired end product of the compounds of the present invention is designated a “protecting group”, unless the context indicates otherwise. The protection of functional groups by such protecting groups, the protecting groups themselves, and their cleavage reactions are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosäuren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide and Derivate” (Chemistry of Carbohydrates: Monosaccharides and Derivatives), Georg Thieme Verlag, Stuttgart 1974. A characteristic of protecting groups is that they can be removed readily (i.e. without the occurrence of undesired secondary reactions) for example by solvolysis, reduction, photolysis or alternatively under physiological conditions (e.g. by enzymatic cleavage).

Salts of compounds of the present invention having at least one salt-forming group may be prepared in a manner known per se. For example, salts of compounds of the present invention having acid groups may be formed, for example, by treating the compounds with metal compounds, such as alkali metal salts of suitable organic carboxylic acids, e.g. the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of the present invention are obtained in customary manner, e.g. by treating the compounds with an acid or a suitable anion exchange reagent. Internal salts of compounds of the present invention containing acid and basic salt-forming groups, e.g. a free carboxy group and a free amino group, may be formed, e.g. by the neutralisation of salts, such as acid addition salts, to the isoelectric point, e.g. with weak bases, or by treatment with ion exchangers.

Salts can be converted in customary manner into the free compounds; metal and ammonium salts can be converted, for example, by treatment with suitable acids, and acid addition salts, for example, by treatment with a suitable basic agent.

Mixtures of isomers obtainable according to the invention can be separated in a manner known per se into the individual isomers; diastereoisomers can be separated, for example, by partitioning between polyphasic solvent mixtures, recrystallisation and/or chromatographic separation, for example over silica gel or by e.g. medium pressure liquid chromatography over a reversed phase column, and racemates can be separated, for example, by the formation of salts with optically pure salt-forming reagents and separation of the mixture of diastereoisomers so obtainable, for example by means of fractional crystallisation, or by chromatography over optically active column materials.

Intermediates and final products can be worked up and/or purified according to standard methods, e.g. using chromatographic methods, distribution methods, (re-) crystallization, and the like.

The following applies in general to all processes mentioned herein before and hereinafter.

All the above-mentioned process steps can be carried out under reaction conditions that are known per se, including those mentioned specifically, in the absence or, customarily, in the presence of solvents or diluents, including, for example, solvents or diluents that are inert towards the reagents used and dissolve them, in the absence or presence of catalysts, condensation or neutralizing agents, for example ion exchangers, such as cation exchangers, e.g. in the H+ form, depending on the nature of the reaction and/or of the reactants at reduced, normal or elevated temperature, for example in a temperature range of from about −100° C. to about 190° C., including, for example, from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

At all stages of the reactions, mixtures of isomers that are formed can be separated into the individual isomers, for example diastereoisomers or enantiomers, or into any desired mixtures of isomers, for example racemates or mixtures of diastereoisomers, for example analogously to the methods described under “Additional process steps”.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofuran or dioxane, liquid aromatic hydrocarbons, such as benzene or toluene, alcohols, such as methanol, ethanol or 1- or 2-propanol, nitriles, such as acetonitrile, halogenated hydrocarbons, such as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, methycyclohexane, or mixtures of those solvents, for example aqueous solutions, unless otherwise indicated in the description of the processes. Such solvent mixtures may also be used in working up, for example by chromatography or partitioning.

The compounds, including their salts, may also be obtained in the form of hydrates, or their crystals may, for example, include the solvent used for crystallization. Different crystalline forms may be present.

The invention relates also to those forms of the process in which a compound obtainable as an intermediate at any stage of the process is used as starting material and the remaining process steps are carried out, or in which a starting material is formed under the reaction conditions or is used in the form of a derivative, for example in a protected form or in the form of a salt, or a compound obtainable by the process according to the invention is produced under the process conditions and processed further in situ.

All starting materials, building blocks, reagents, acids, bases, dehydrating agents, solvents and catalysts utilized to synthesize the compounds of the present invention are either commercially available or can be produced by organic synthesis methods known to one of ordinary skill in the art (Houben-Weyl 4^{th} Ed. 1952, Methods of Organic Synthesis, Thieme, Volume 21).

Certain of the intermediates used in the processes as hereinbefore described are novel per se. Therefore, according to a further aspect of the invention there is provided a compound of formula VII;

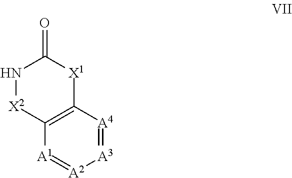

in which X¹, X², A¹, A², A³ and A⁴ are each as hereinbefore defined;

and isomers thereof;

in free form or in salt form.

Referring to the examples that follow, compounds of the preferred embodiments were synthesized using the methods described herein, or other methods, which are known in the art.

It should be understood that the organic compounds according to the preferred embodiments may exhibit the phenomenon of tautomerism. As the chemical structures within this specification can only represent one of the possible tautomeric forms, it should be understood that the preferred embodiments encompasses any tautomeric form of the drawn structure.

The following examples are intended to illustrate the invention and are not to be construed as being limitations thereon. Temperatures are given in degrees centigrade. If not mentioned otherwise, all evaporations are performed under reduced pressure, preferably between about 15 mm Hg and 100 mm Hg (=20-133 mbar). The structure of final products, intermediates and starting materials was confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

In addition various trade reagents and materials available from have been utilized. Such reagents and materials include IST PE-AX/SCX-2 and SCX-2 cartridges and can be readily obtained from the suppliers indicated.

### General Conditions:

¹H-NMR: Spectra were run on either a Bruker AVANCE 400 (400 MHz) spectrometer or on a Bruker AVANCE 500 (500 MHz) NMR spectrometer using ICON-NMR. Spectra are measured at 298K and are referenced using the solvent peak, chemical shifts (δ-values) are reported in ppm, where included, coupling constants (J) are given in Hz, spectra splitting pattern are designated as singlet (s), doublet (d), triplet (t), quadruplet (q), multiplet or more overlapping signals (m), broad signal (br), (app) apparent and solvent is given in parentheses.

MS: These are either Agilent 1100 HPLC/Micromass Platform Mass Spectrometer combinations or Waters Acquity HPLC with SQD Mass Spectrometer or Waters Alliance HT HPLC system equipped with a MS detector Waters MicromassZQ or Waters Micromass Plattform LCZ system. Mass spectra are run on LC-MS systems using electrospray ionization. [M+H]+ refers to mono-isotopic molecular weights.

The various starting materials, intermediates, and compounds of the preferred embodiments may be isolated and purified, where appropriate, using conventional techniques such as precipitation, filtration, crystallization, evaporation, distillation, catch and release, and chromatography. Unless otherwise stated, all starting materials are obtained from commercial suppliers and used without further purification. Salts may be prepared from compounds by known salt-forming procedures.

Where a mixture of products was obtained that was inseparable by conventional techniques, these were separated using Supercritical Fluids Chromatography (SFC). The general conditions for screening and preparative chiral separations by SFC were as follows:

Approximately 1.0 mg of sample is dissolved in 1.0 ml ethanol and screened on a Thar Minigram SFC system using the following chromatographic conditions:

### Columns:

### Chiralpak AD-H, 250×10 mm id, 5 μm

### Chiralpak AS-H, 250×10 mm id, 5 μm

### Chiralpak IC, 250×10 mm id, 5 μm

### Chiralcel OD-H, 250×10 mm id, 5 μm

### Chiralcel OJ-H, 250×10 mm id, 5 μm

### Mobile Phase A:

Methanol (with the addition of 0.1% v/v DEA or TFA depending on the compound)

### Mobile Phase B:

2-Propanol (with the addition of 0.1% v/v DEA or TFA depending on the compound)

Mobile Phase C: CO₂

Screen 1 Conditions:

| | | | | | |
|---|---|---|---|---|---|
| Gradient: | Time 0-3 min | 10% | A | 90% | C |
| | Time 3-10 min | 10-50% | A | 90-50% | C |
| | Time 10-13 min | 50% | A | 50% | C |
| | Time 13-14 min | 50-10% | A | 50-90% | C |
| | Time 14-15 min | 10% | A | 90% | C |

Screen 2 Conditions:

As screen 1 but with mobile phase B replacing mobile phase A

| | |
|---|---|
| Detection: | UV @ 220 nm |
| Flow rate: | 10 ml/min |
| Sample concentration: | 1.0 mg in 1 ml ethanol |
| Injection volume: | 30 μl |

The resulting chromatograms are examined for the best resolution of the sample. The optimum column and modifier are identified.

Optimisation of an isocratic method is then carried out to find a method suitable for the preparative separation.

The preparative separation is carried out on one of the five columns listed above and with either methanol or 2-propanol (with addition of DEA or TFA if necessary for optimum separation) and CO₂.

The total amount of sample is dissolved in ethanol, and multiple injections are carried out until all the sample solution is used. Injection volumes range from 50 μl to 200 μl depending on sample concentration and limit of loading on the column.

For the examples below as well as throughout the application, the following abbreviations have the following meanings. If not defined, the terms have their generally accepted meanings.

### ABBREVIATIONS

aq. aqueous
DCM dichloromethane
DIPEA N,N-diisopropylethylamine
DMF N,N-dimethylformamide
Et₂O diethylether
EtOAc ethyl acetate
h hour
HATU 2-(1H-7-azabenzotriazol-1-yl)-1,1,3,3-tetramethyl uronium hexafluorophosphate
LDA lithium diisopropylamide
MeCN acetonitrile
MeOH methanol
min minute
ppt precipitate
Rt retention time
RT room temperature
sat. saturated
^{t}BuOH tert-butanol
TFA trifluoroacetic acid
SEM-Cl 2-(trimethylsilyl)ethoxymethyl chloride

If not indicated otherwise, the analytical HPLC conditions were as follows:

### Method LowpH_v002

| | |
|---|---|
| Column | Phenomenex Gemini C18 50 × 4.6 mm, 3.0 μm |
| Column Temperature | 50° C. |
| Eluents | A: H₂O, B: methanol, both containing 0.1% TFA |
| Flow Rate | 1.0 ml/min |
| Gradient | 5% to 95% B in 2.0 min, 0.2 min 95% B |

Method 2minLC_v002

| | |
|---|---|
| Column | Waters BEH C18 50 × 2.1 mm, 1.7 μm |
| Column Temperature | 50° C. |
| Eluents | A: H₂O, B: methanol, both containing 0.1% TFA |
| Flow Rate | 0.8 ml/min |
| Gradient | 0.20 min 5% B; 5% to 95% B in 1.30 min, |
| | 0.25 min 95% B |

Method 2minLC_{—}30_v002

| | |
|---|---|
| Column | Waters BEH C18 50 × 2.1 mm, 1.7 μm |
| Column Temperature | 50° C. |
| Eluents | A: H₂O, B: methanol, both containing 0.1% TFA |
| Flow Rate | 0.8 ml/min |
| Gradient | 0.25 min 30% B; 30% to 95% B in 1.00 min, |
| | 0.25 min 95% B |

### PREPARATION OF EXAMPLES

### Example 1.1

### Trans-2-Chloro-N-[4-(6-chloro-2-oxo-2,3-dihydro-indol-1-ylmethyl)-cyclohexyl]-5-trifluoromethyl-benzamide

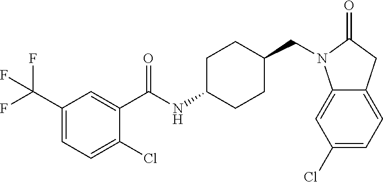

To a stirring solution of 6-chloro-2-oxindole (commercially available) (34.2 mg, 0.204 mmol) in DMF (1 ml) was added NaH (8.16 mg, 0.204 mmol). The mixture was left to stir for 1.5 hours at RT and then treated with toluene-4-sulfonic acid 4-(2-chloro-5-trifluoromethyl-benzoylamino)-cyclohexylmethyl ester (Intermediate B) (50 mg, 0.102 mmol) in DMF (1 ml). After stirring at 50° C. overnight, the reaction mixture was partitioned between EtOAc and water. The aqueous portion was separated and extracted with EtOAc (3×20 ml). The combined organic extracts were washed with water, brine, dried (MgSO₄) and concentrated in vacuo to yield an orange oil. Purification of the oil by preparative LC-MS eluting with water:MeCN (0.1% TFA) afforded the title compound as a light purple solid; LC-MS Rt 1.37 mins; MS m/z 485.2 [M+H]⁺; Method 2minLC_{—}30_v002.

¹H NMR (400 MHz, CDCl3) δ7.90 (1H, s), 7.60 (1H, dd), 7.53 (1H, d), 7.17 (1H, d), 7.03 (1H, dd), 6.82 (1H, d), 5.99 (1H, d), 4.00 (1H, m), 3.56 (4H, m), 2.19 (2H, m), 1.81 (3H, m), 1.27 (2H, m).

The compounds of the following tabulated Examples (Table 1) were prepared by a similar method to that of Example 1.1 using the appropriate tosylate and oxindole starting compounds, the preparations of which are described hereinafter (see ‘Intermediates’ section.)

TABLE 1

| | | | Retention Time | |
|---|---|---|---|---|
| | | | (min), [M + H]⁺ | |
| | | | (Method | |
| Ex. | Structure | Name | 2minLC_30_v002) | ¹H NMR |
| 1.2 | 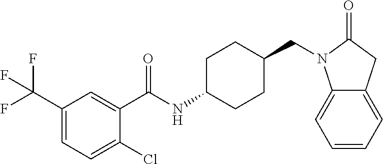 | Trans-2-chloro-N- [4-(2-oxo-2,3- dihydro-indol-1- ylmethyl)- cyclohexyl]-5- trifluoromethyl- benzamide | Rt 1.3 min [M + H]⁺ 451.2 | (400 MHz, CDCl3) δ 7.90 (1H, d), 7.60 (1H, dd), 7.53 (1H, d), 7.38 (2H, m), 7.07 (1H, t), 6.85 (1H, d), 5.98 (1H, d), 4.00 (1H, m), 3.60 (2H, m), 3.58 (2H, m), 2.19 (2H, m), 1.86 (3H, m), 1.26 (4H, m). |
| 1.3 | 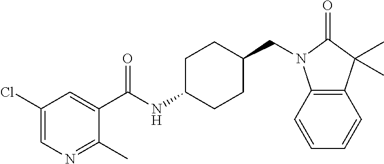 | Trans-5-chloro-N- [4-(3,3-dimethyl-2- oxo-2,3-dihydro- indol-1-ylmethyl)- cyclohexyl]-2- methyl-icotinamide | Rt 1.29 min [M + H]⁺ 426.2 | (400 MHz, CDCl3) δ 8.5 (1H, s), 7.7 (1H, s), 7.2 (2H, m), 7.05 (1H, t), 6.85 (1H, d), 5.75 (1H, br), 3.9 (1H, m), 3.6 (2H, d), 2.65 (3H, s), 2.15 (2H, br), 1.8 (3H, m), 1.4 (6H, s), 1.25 (4H, m). |

### Example 2.1

### Trans-2-chloro-N-[4-(6-chloro-3,3-difluoro-2-oxo-2,3-dihydro-indol-1-ylmethyl)-cyclohexyl]-5-trifluoromethyl-benzamide

A solution of 6-chloro-3,3-difluoro-1,3-dihydro-indol-2-one (Intermediate F) (108 mg, 2 eq) in dry DMF (2 ml) was treated with NaH (22 mg, 2 eq) and the vial was flushed with N₂. To this was added trans-methanesulfonic acid 4-(2-chloro-5-trifluoromethyl-benzoylamino)-cyclohexyl methyl ester (Intermediate C) (110 mg, 0.265 mmol) and the reaction heated to 50° C. for 2 days. After cooling to RT, the mixture was diluted with EtOAc/H₂O (20 ml) and transferred to a separating funnel. The organic layer was separated and washed with brine, dried (MgSO₄) and concentrated in vacuo. The crude product was purified by chromatography on silica eluting in a 0% to 20% EtOAc in iso-hexane to afford the title product; LC-MS Rt 2.64 mins; [M+H]+ 521. Method LowpH_v002. ¹H NMR (400 MHz, DMSO) δ 8.48 (1H, d), 7.80 (1H, dd), 7.72 (3H, d), 7.55 (1H, s), 7.3 (1H, d), 3.70 (1H, m), 3.58 (2H, d), 1.9 (2H, m), 1.65-1.8 (3H, m), 1.1-1.3 (4H, m).

### Example 2.2

### Trans-2-chloro-N-[4-(3,3-dimethyl-2-oxo-2,3-dihydro-pyrrolo[2,3-b]pyridin-1-ylmethyl)-cyclohexyl]-5-trifluoromethyl-benzamide

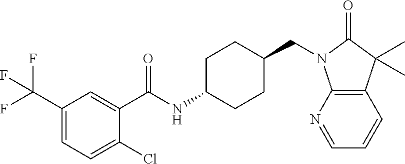

A solution of 3,3-dimethyl-1,3-dihydro-pyrrolo[2,3-b]pyridin-2-one (Intermediate G) (150 mg, 2 eq) in dry DMF (2 ml) was treated with NaH (37 mg, 2 eq) and the vial was flushed with N₂. To this was added trans-methanesulfonic acid 4-(2-chloro-5-trifluoromethyl-benzoylamino)-cyclohexyl methyl ester (Intermediate C) (191 mg, 0.462 mmol) and the reaction heated to 50° C. overnight. After cooling to RT, the mixture was diluted with EtOAc and transferred to a separating funnel. The organic layer was separated and washed with brine, dried (MgSO₄) and concentrated in vacuo. The crude product was purified by chromatography on silica eluting in a 0% to 30% EtOAc in iso-hexane to afford the title product; LC-MS Rt=2.54 min; [M+H]+ 480.37. Method LowpH_v002. ¹H NMR (400 MHz, DMSO) δ 8.48 (1H, d), 8.15 (1H, d), 7.80 (1H, d), 7.72 (3H, d), 7.05 (1H, t), 3.70 (1H, m), 3.58 (2H, d), 2-1.8 (3H, m), 1.65 (2H, d), 1.3 (6H, s), 1.1-1.3 (4H, m).

### Example 2.3

### Trans-5-chloro-N-[4-(3,3-dimethyl-2-oxo-2,3-dihydro-pyrrolo[2,3-b]pyridin-1-ylmethyl)-cyclohexyl]-2-methyl-nicotinamide

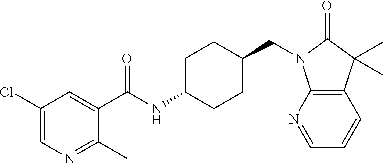

A solution of 3,3-dimethyl-1,3-dihydro-pyrrolo[2,3-b]pyridin-2-one (Intermediate G) (50 mg, 1 eq) in dry DMF (1 ml) was treated with NaH (12.5 mg, 1 eq) was added and the vial was then flushed with N₂. To this was trans-methanesulfonic acid 4-[(5-chloro-2-methyl-pyridine-3-carbonyl)-amino]-cyclohexylmethyl ester (Intermediate D) (83 mg, 0.231 mmol) and the reaction heated to 50° C. overnight. After cooling to RT, the mixture was diluted with EtOAc and transferred to a separating funnel. The organic layer was separated and washed with brine, dried (MgSO₄) and concentrated in vacuo. The crude product was purified by preparative LC-MS to afford the title product; LC-MS Rt=2.38; [M+H]+ 427.46. Method LowpH_v002.

### Example 2.4

### Trans-2-chloro-N-[4-(2-oxo-2,3-dihydro-pyrrolo[2,3-b]pyridin-1-ylmethyl)-cyclohexyl]-5-trifluoromethyl-benzamide

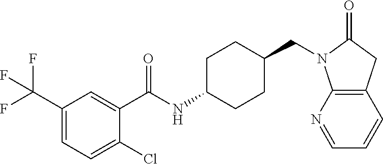

1,3-Dihydro-pyrrolo[2,3-b]pyridin-2-one (Intermediate G step 2) (162 mg, 2 eq) in dry DMF (2 ml) was treated with NaH (48.5 mg, 2 eq) was added and the vial was then flushed with N₂. To this was then added trans-methanesulfonic acid 4-(2-chloro-5-trifluoromethyl-benzoylamino)-cyclohexyl methyl ester (Intermediate C) (250 mg, 0.362 mmol) and the reaction heated to 50° C. overnight. After cooling to RT, the mixture was diluted with EtOAc/H₂O (20 ml) and transferred to a separating funnel. The organic layer was separated and washed with brine, dried (MgSO₄) and concentrated in vacuo. The crude product was purified by chromatography on silica eluting in a 0% to 100% EtOAc in iso-hexane followed by preparative LC-MS to afford the title product; LC-MS Rt 2.45 min; [M+H]+ 452.4; Method LowpH_v002. ¹H NMR (400 MHz, DMSO) δ 8.45 (1H, d), 8.15 (1H, d), 7.8 (1H, d), 7.75 (2H, d), 7.6 (1H, d), 7.0 (1H, t), 3.70 (1H, m), 3.58 (2H, s), 3.55 (2H, d), 1.9 (2H, m), 1.8 (1H, m), 1.65 (2H, m), 1.1-1.3 (4H, m).

### Example 2.5

### Trans-2-chloro-N-{-4-((5′-fluoro-2′-oxospiro[cyclopropane-1,3′-indoline]-1′-yl)methyl)cyclohexyl}-5-(trifluoromethyl)benzamide

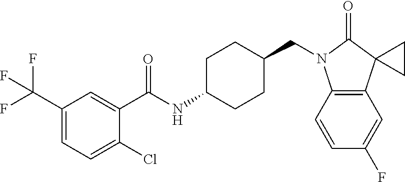

5′-Fluorospiro[cyclopropane-1,3′-indolin]-2′-one (Intermediate H) (67 mg, 0.378 mmol) in dry DMF (3 ml) was treated with NaH (60% in mineral oil) (15.12 mg, 0.378 mmol) under N₂ and the contents left stirring for ˜10 mins. After this time, trans-methanesulfonic acid 4-(2-chloro-5-trifluoromethyl-benzoylamino)-cyclohexyl methyl ester (Intermediate C) (105 mg, 0.253 mmol) was added and the solution heated to 70° C. for 2.5 h. After cooling to RT overnight, the mixture was diluted with EtOAc/H2O (40 ml) and transferred to a separating funnel. The organic layer was separated and washed with brine, dried (MgSO₄), and concentrated in vacuo to give a light brown oil. The crude oil was chromatographed on silica eluting with a gradient of 0%-30% EtOAc/iso-hexane followed by an isocratic gradient of 30% EtOAc/iso-hexane to afford the title compound as a white solid; LC-MS Rt=1.36 mins; [M+H]+ 495.3; Method=2minLC_{—}30_v002. ¹H NMR (400 MHz, CDCl3). δ 7.90 (1H, s), 7.55 (1H, d), 7.50 (1H, d), 6.95 (1H, m), 6.80 (1H, m), 6.60 (1H, m), 5.95 (1H, d), 4.0 (1H, m), 3.65 (2H, d), 2.20 (2H, m), 1.85 (5H, m), 1.55 (2H, m), 1.25 (4H, m).

The compounds of the following tabulated Examples (Table 2) were prepared by a similar method to that of Example 2.1 from trans-methanesulfonic acid 4-(2-chloro-5-trifluoromethyl-benzoylamino)-cyclohexyl methyl ester (Intermediate C) or trans-methanesulfonic acid 4-[(5-chloro-2-methyl-pyridine-3-carbonyl)-amino]-cyclohexylmethyl ester (Intermediate D) and the appropriate oxindole/azaoxindole (the preparations of which are described in the ‘Preparation of Intermediates’ section.

### Example 2.33

### Trans-5-Chloro-N-[4-(-6-chloro-3-fluoro-3-methyl-2-oxo-2,3-dihydro-indol-1-ylmethyl)-cyclohexyl]-2-methyl-nicotinamide

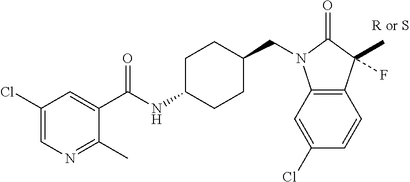

An Enantiomer of 6-chloro-3-fluoro-3-methylindolin-2-one (Intermediate KF) (86 mg, 0.431 mmol) was dissolved in DMF (2.5 ml) and treated with NaH (18.97 mg, 0.474 mmol) added in one portion. The mixture was stirred for 10 minutes and trans-methanesulfonic acid 4-[(5-chloro-2-methyl-pyridine-3-carbonyl)-amino]-cyclohexylmethyl ester (Intermediate D) (140 mg, 0.388 mmol) was added in one portion and the mixture was heated at 50° C. overnight. The solvent was removed in vacuo and the residue was partitioned between DCM and water/brine. The organic portion was removed using a phase separator and concentrated in vacuo. Purification by reverse phase chromatography afforded the title product; ¹H NMR (400 MHz, DMSO) δ 8.53 (1H, d), 8.38 (1H, d), 7.78 (1H, d), 7.60 (1H, dd), 7.38 (1H, s), 7.18 (1H, dd), 3.61-3.77 (1H, m), 3.54 (2H, d), 2.46 (3H, s), 1.82-98 (2H, m), 1.6-1.8 (6H, m), 1.04-1.3 (4H, m)

¹⁹F NMR 148.76 ppm.

### Example 2.46

### Trans-5-Chloro-N-[4-(3-ethyl-2-oxo-2,3-dihydro-benzoimidazol-1-ylmethyl)-cyclohexyl]-2-methyl-nicotinamide

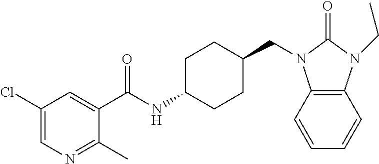

A stirred solution of 1-ethyl-1,3-dihydro-benzoimidazol-2-one (Intermediate RE) (44.9 mg, 0.277 mmol) in DMF (2 ml) under N₂ was treated with NaH (60% in oil) (13.30 mg, 0.333 mmol) and the reaction mixture was stirred at RT for 10 minutes. Trans-methanesulfonic acid 4-[(5-chloro-2-methyl-pyridine-3-carbonyl)-amino]-cyclohexylmethyl ester (Intermediate D) (100 mg, 0.277 mmol) was added and the mixture was heated at 80° C. for 3 hr 30 mins. The solvent was removed in vacuo and purification by chromatography on silica eluting with EtOAc/iso-hexane afforded the title product; LC-MS Rt=2.53; [M+H]+ 427.25. Method LowpH_v002. ¹H NMR (400 MHz, DMSO) δ 8.53 (1H, s), 8.38 (1H, d), 7.78 (1H, s), 7.21 (2H, m), 7.06 (2H, m), 3.88 (2H, m), 3.70 (3H, m), 2.46 (3H, s), 1.88 (2H, m), 1.76 (1H, m), 1.67 (2H, m), 1.20 (7H, m).

### Example 2.47

### Trans-5-Chloro-N-[4-(3-isobutyl-2-oxo-2,3-dihydro-benzoimidazol-1-ylmethyl)-cyclohexyl]-2-methyl-nicotinamide

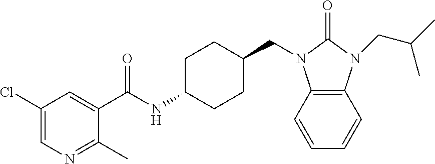

1-Isobutyl-1,3-dihydro-benzoimidazol-2-one (Intermediate RO) (52.7 mg, 0.277 mmol) in DMF (3 ml) was treated with NaH (60% in oil) (13.30 mg, 0.333 mmol) and stirred at RT for 10 minutes. Trans-methanesulfonic acid 4-[(5-chloro-2-methyl-pyridine-3-carbonyl)-amino]-cyclohexylmethyl ester (Intermediate D) (100 mg, 0.277 mmol) was added and the mixture was heated at 75° C. 3 hr 30 mins. The solvent was removed in vacuo and the reaction mixture was partitioned between EtOAc (˜40 ml) and water (˜10 ml). The aqueous portion was separated and extracted with EtOAc (20 ml). The combined organic portions were washed with sat. brine and dried (MgSO₄). The solvent was removed in vacuo and purification by chromatography on silica eluting with EtOAc/iso-hexane afforded the title product; LC-MS Rt=2.64; M+H]+ 455.33. Method LowpH_v002. ¹H NMR (400 MHz, DMSO) δ 8.53 (1H, d), 8.37 (1H, d), 7.78 (1H, s), 7.20 (2H, m), 7.05 (2H, m), 3.67 (5H, m), 2.46 (3H, s), 2.11 (1H, m), 1.88 (2H, m), 1.78 (1H, m), 1.67 (2H, m), 1.17 (4H, m), 0.88 (6H, d).

TABLE 2

| | | | Rention Time | |
|---|---|---|---|---|
| | | | (Method | |
| | | | LowpH_v002) | |
| | | | unless | |
| | | | otherwise | |
| Ex. | Structure | Name | specified | NMR |
| 2.6 | 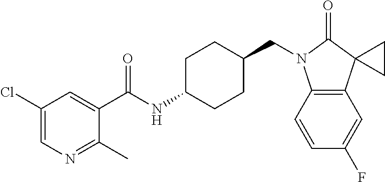 | Trans-5-chloro-N-[-4- ((5′-fluoro-2′-oxospiro [cyclopropane-1,3′- indoline]-1′-yl)methyl) cyclohexyl]-2-methyl nicotinamide | Rt = 5.08 min; [M + H]⁺ 442.2 Method 2minLC_30_v002 | 1H NMR (400 MHz, CDCl3) δ 8.50 (1H, s), 7.65 (1H, s), 6.95 (1H, t), 6.80 (1H, m), 6.60 (1H, d), 5.60 (1H, d), 3.95 (1H, m), 3.65 (2H, d), 2.65 (3H, s), 2.15 (2H, m), 1.85 (5H, m), 1.50 (2H, d), 1.25 (4H, m). |
| 2.7 | 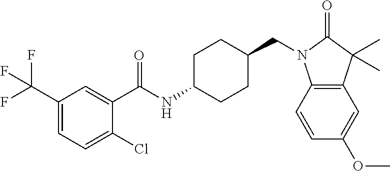 | Trans-2-Chloro-N-[4-(5- methoxy-3,3-dimethyl-2- oxo-2,3-dihydro-indol-1- ylmethyl)-cyclohexyl]-5- trifluoromethyl- benzamide | Rt = 1.12 min; [M + H]⁺ 509.4 Method 2minLC_30_v002 | 1H NMR (400 MHz, CDCl3) δ 7.9 (1H, s), 7.60 (1H, d), 7.50 (1H, d), 6.85 (1H, s), 6.75 (2H, m), 6.0 (1H, d), 3.95 (1H, m), 3.80 (3H, s), 3.55 (2H, d), 2.20 (2H, m), 1.85 (3H, m), 1.40 (6H, s), 1.25 (4H, m). |
| 2.8 | 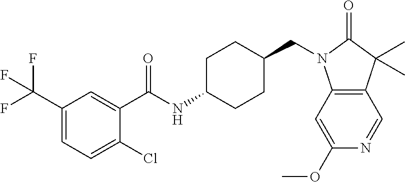 | Trans-2-Chloro-N-[4-(6- methoxy-3,3-dimethyl-2- oxo-2,3-dihydro- pyrrolo[3,2-c] pyridin-1-ylmethyl)- cyclohexyl]-5- trifluoromethyl- benzamide | Rt 1.04 min; [M + H]⁺ 510.3 Method 2minLC_30_v002 | 1H NMR (400 MHz, CDCl3) δ 7.9 (2H, m), 7.60 (1H, d), 7.55 (1H, d), 6.20 (1H, s), 5.95 (1H, d), 3.95 (4H, m), 3.55 (2H, d), 2.20 (2H, m), 1.80 (3H, m), 1.40 (6H, s), 1.25 (4H, m). |
| 2.9 | 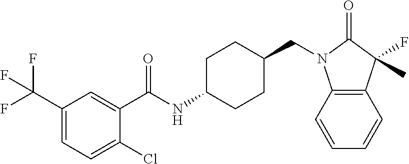 | Trans-2-Chloro-N-[4- ((R)-3-fluoro-3-methyl- 2-oxo-2,3-dihydro- indol-1-ylmethyl)- cyclohexyl]-5- trifluoromethyl- benzamide | Rt = 2.55 min; [M − F]⁺ 463.42 | 1H NMR (400 MHz, DMSO) δ 8.47 (1H, d), 7.81 (1H, dd), 7.73 (2H, app d), 7.56 (1H, d), 7.43 (1H, t), 7.08-7.25 (2H, m), 3.69 (1H, m), 3.53 (2H, d), 1.83-2.0 (2H, m), 1.6-1.8 (6H, m), 1.05-1.3 (4H, m) |
| 2.10 | 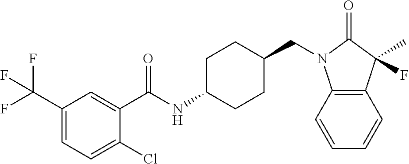 | Trans-2-Chloro-N-[4- ((S)-3-fluoro-3-methyl- 2-oxo-2,3-dihydro- indol-1-ylmethyl)- cyclohexyl]-5- trifluoromethyl- benzamide | Rt = 2.55 min; [M + H]⁺ 483.4 | 1H NMR (400 MHz, DMSO) δ 8.47 (1H, d), 7.79 (1H, dd), 7.73 (2H, app d), 7.58 (1H, d), 7.43 (1H, t), 7.08-7.15 (2H, m), 3.69 (1H, app m), 3.53 (2H, d), 1.85-2.0 (2H, m), 1.60-1.8 (6H, m), 1.05-1.3 (4H, m) |
| 2.11 | 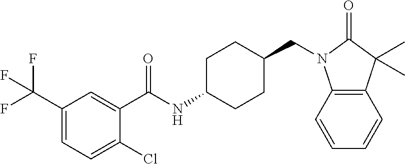 | Trans-2-Chloro-N-[4- (3,3-dimethyl-2-oxo- 2,3-dihydro-indol-1- ylmethyl)-cyclohexyl]- 5-trifluoromethyl- benzamide | Rt = 1.38 min; [M + H]⁺ 479.2 Method 2minLC_30_v002 | 1H NMR (400 MHz, CDCl3) δ 7.9 (1H, s), 7.6 (1H, d), 7.5 (1H, d), 7.2 (2H, m), 7.1 (1H, d), 6.85 (1H, d), 5.95 (1H, d), 4.0 (1H, m), 3.6 (2H, d), 2.2 (2H, br), 1.85 (3H, m), 1.4 (6H, s), 1.25 (4H, m). |
| 2.12 | 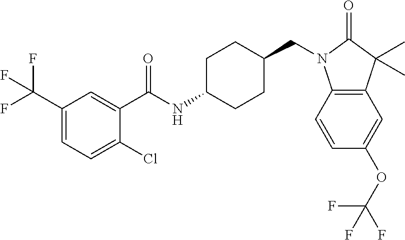 | Trans-2-Chloro-N-[4- (3,3-dimethyl-2-oxo-5- trifluoromethoxy-2,3- dihydro-indol-1- ylmethyl)-cyclohexyl]- 5-trifluoromethyl- benzamide | Rt = 1.35 min; [M + H]⁺ 563.3 Method 2minLC_30_v002 | 1H NMR (400 MHz, CDCl3) δ 7.9 (1H, s), 7.4 (1H, d), 7.5 (1H, d), 7.1 (2H, m), 6.8 (1H, d), 5.95 (1H, d), 4.0 (1H, m), 3.6 (2H, d), 2.2 (2H, m), 1.8 (3H, m), 1.4 (6H, s), 1.3 (4H, m). |
| 2.13 | 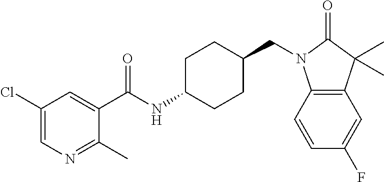 | Trans-5-Chloro-N-[4-(5- fluoro-3,3-dimethyl-2- oxo-2,3-dihydro-indol- 1-ylmethyl)- cyclohexyl]-2-methyl- nicotinamide | Rt = 1.29 min; [M + H]⁺ 444.3 Method 2minLC_30_v002 | 1H NMR (400 MHz, CDCl3) δ 8.50 (1H, s), 7.65 (1H, s), 6.95 (2H, m), 6.75 (1H, m), 5.6 (1H, br), 3.95 (1H, m), 3.55 (2H, d), 2.60 (3H, s), 2.15 (2H, m), 1.80 (3H, m), 1.40 (6H, s), 1.20 (4H, m). |
| 2.14 | 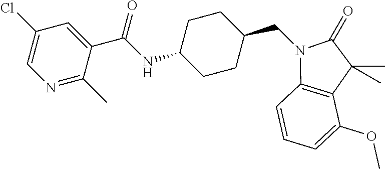 | Trans-5-Chloro-N-[4-(4- methoxy-3,3-dimethyl-2- oxo-2,3-dihydro-indol-1- ylmethyl)-cyclohexyl]-2- methyl-nicotinamide | Rt = 2.66 min; [M + H]⁺ 456.44 | 1H NMR (400 MHz, CDCl3) δ 8.48 (1H, s), 7.75 (1H, s), 7.2 (1H, t), 6.65 (1H, d), 6.5 (1H, d), 5.9 (1H, br), 3.9 (1H, m), 3.85 (3H, s), 3.55 (2H, d), 2.70 (3H, s), 2.15 (2H, m), 1.80 (3H, m), 1.45 (6H, s), 1.25 (4H, m). |
| 2.15 | 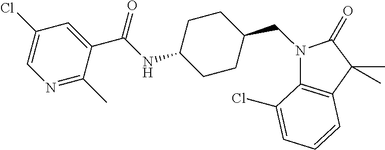 | Trans-5-Chloro-N-[4-(7- chloro-3,3-dimethyl-2- oxo-2,3-dihydro-indol- 1-ylmethyl)-cyclohexyl]- 2-methyl-nicotinamide | Rt = 2.6 min; [M + H]⁺ 460.38 | 1H NMR (400 MHz, TFA: TFA acid set to 12.00 ppm) δ 12.95 (1H, s), 12.75 (1H, s), 11.55 (1H, d), 11.45 (1H, d), 11.4 (1H, t), 8.35 (2H, d), 8.25 (1H, m), 7.15 (3H, s), 6.4 (2H, s), 6.25 (2H, s), 6.15 (2H, s), 5.65 (8H, m), 5.3 (2H, s) |
| 2.16 | 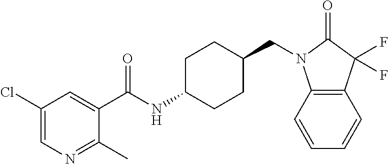 | Trans-5-Chloro-N-[4- (3,3-difluoro-2-oxo-2,3- dihydro-indol-1- ylmethyl)-cyclohexyl]- 2-methyl-nicotinamide | Rt = 2.43 min; [M + H]⁺ 434.4 | 1H NMR (400 MHz, DMSO) δ 8.53 (1H, d), 8.39 (1H, d), 7.78 (1H, d), 7.71 (1H, d) 7.62 (1H, t), 7.34 (1H, d), 7.24 (1H, t), 3.62-3.78 (1H, m), 3.58 (2H, d), 2.46 (3H, s), 1.8- 2.0 (2H, m), 1.6-1.8 (3H, m), 1.05-1.3 (4H, m) |
| 2.17 | 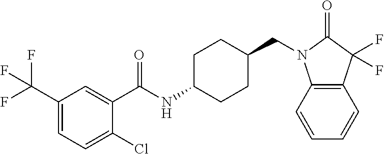 | Trans-2-Chloro-N-[4- (3,3-difluoro-2-oxo-2,3- dihydro-indol-1- ylmethyl)-cyclohexyl]- 5-trifluoromethyl- benzamide | Rt = 2.56 min; [M + H]⁺ 487.3 | 1H NMR (400 MHz, DMSO) δ 8.48 (1H, d), 7.80 (1H, dd), 7.72 (3H, app t), 7.62 (1H, t), 7.33 (1H, d), 7.24 (1H, t), 3.70 (1H, m), 3.58 (2H, d), 1.85-2.00 (2H, m), 1.65-1.8 (3H, m), 1.1-1.3 (5H, m). |
| 2.18 | 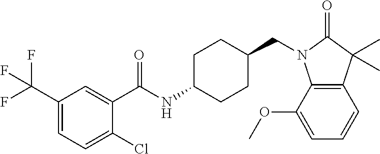 | Trans-2-Chloro-N-[4-(7- methoxy-3,3-dimethyl-2- oxo-2,3-dihydro-indol- 1-ylmethyl)-cyclohexyl]- 5-trifluoromethyl- benzamide | Rt = 2.64 min; [M + H]⁺ 509.45 | 1H NMR (400 MHz, DMSO) δ 8.45 (1H, d), 7.79 (1H, dd), 7.70-7.78 (3H, m), 6.95-7.08 (3H, m), 3.85 (3H, s), 3.6-3.8 (3H, m), 1.85-2.0 (2H, m), 1.6-1.7 (3H, m), 1.0-1.3 (11H, m) |
| 2.19 | 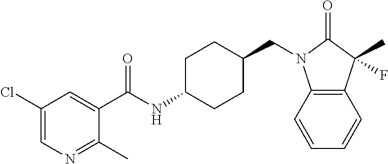 | Trans-5-Chloro-N-[4- ((R)-3-fluoro-3-methyl- 2-oxo-2,3-dihydro- indol-1-ylmethyl)- cyclohexyl]-2-methyl- nicotinamide | Rt = 2.42 min; [M + H]⁺ 430.5 | 1H NMR (400 MHz, DMSO) δ 8.53 (1H, d), 8.35 (1H, d), 7.78 (1H, d), 7.56 (1H, d), 7.43 (1H, t), 7.1-7.24 (2H, m), 3.69 (1H, m), 3.53 (2H, d), 2.46 (3H, s), 1.8-2.0 (2H, m), 1.6-1.8 (6H, m), 1.05-1.3 (4H, m). |
| 2.20 | 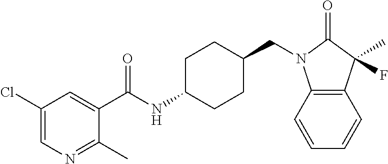 | Trans-5-Chloro-N-[4- ((S)-3-fluoro-3-methyl- 2-oxo-2,3-dihydro- indol-1-ylmethyl)- cyclohexyl]-2-methyl- nicotinamide | Rt = 2.41 min; [M + H]⁺ 430.5 | 1H NMR (400 MHz, DMSO) δ 8.53 (1H, d), 8.38 (1H, d), 7.78 (1H, d), 7.56 (1H, d), 7.43 (1H, t), 7.10-7.23 (2H, m), 3.69 (1H, m), 2.46 (3H, s), 1.8-1.97 (2H, m), 1.6-1.8 (6H, m), 1.05-1.3 (4H, m). |
| 2.21* | 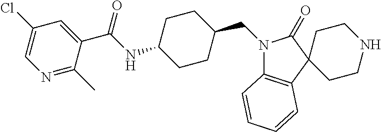 | Trans-5-chloro-2- methyl-N-(-4-((2- oxospiro[indoline- 3,4′-piperidine]-1- yl)methyl) cyclohexyl) nicotinamide | Rt = 1.99 min; [M + H]⁺ 467.48 | 1H NMR (400 MHz, DMSO) δ 8.55 (1H, d), 8.38 (1H, d), 7.78 (1H, d), 7.56 (1H, d), 7.25 (1H, t), 7.0-7.1 (2H, m), 3.69 (1H, m), 3.55 (2H, d), 3.1 (2H, m), 2.9 (2H, m), 2.45 (3H, s), 1.9 (2H, m), 1.7 (6H, m), 1.5 (2H, m) 1.15 (4H, m). |
| 2.22 | 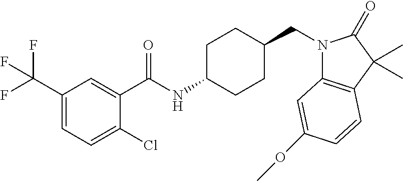 | Trans-2-Chloro-N-[4-(6- methoxy-3,3-dimethyl-2- oxo-2,3-dihydro-indol- 1-ylmethyl)-cyclohexyl]- 5-trifluoromethyl- benzamide | Rt = 1.15 min; [M + H]⁺ 509.2 Method 2minLC_30_v002 | 1H NMR (400 MHz, CDCl3) δ 7.90 (1H, s), 7.60 (1H, d), 7.50 (1H, d), 7.10 (1H, d), 6.55 (1H, d), 6.45 (1H, s), 5.95 (1H, d), 3.95 (1H, m), 3.80 (3H, s), 3.55 (2H, d), 2.15 (2H, d), 1.80 (3H, m), 1.35 (6H, s), 1.25 (4H, m). |
| 2.23 | 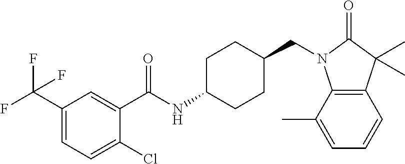 | Trans-2-Chloro-5- trifluoromethyl-N-[4- (3,3,7-trimethyl-2-oxo- 2,3-dihydro-indol-1- ylmethyl)-cyclohexyl]- benzamide | Rt = 2.71 min; [M + H]⁺ 493.26 | 1H NMR (400 MHz, CDCl3) δ 7.9 (1H, s), 7.6 (1H, d), 7.5 (1H, d), 7.1 (1H, d), 6.95 (2H, m), 5.95 (1H, d), 4.0 (1H, m), 3.85 (2H, d), 2.55 (3H, s), 2.2 (2H, d), 1.8 (3H, m), 1.4 (6H, s), 1.25 (4H, m). |
| 2.24 | 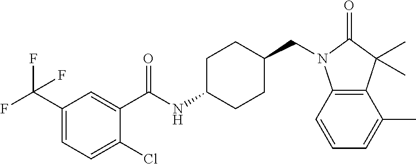 | Trans-2-Chloro-5- trifluoromethyl-N-[4- (3,3,4-trimethyl-2-oxo- 2,3-dihydro-indol-1- ylmethyl)-cyclohexyl]- benzamide | Rt = 2.71 min; [M + H]⁺ 493.32 | 1H NMR (400 MHz, DMSO) δ 8.45 (1H, d), 7.8 (1H, d), 7.7 (2H, d), 7.15 (1H, t), 6.9 (1H, d), 6.8 (1H, d), 3.7 (1H, m), 3.5 (2H, d), 2.35 (3H, s), 1.9 (2H, m), 1.7 (3H, m), 1.35 (6H, s), 1.15 (4H, m). |
| 2.25 | 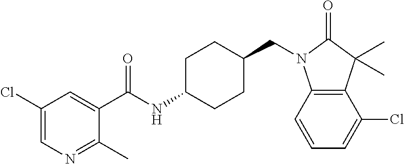 | Trans-5-Chloro-N-[4-(4- chloro-3,3-dimethyl-2- oxo-2,3-dihydro-indol- 1-ylmethyl)-cyclohexyl]-2- methyl-nicotinamide | Rt = 2.67 min; [M + H]⁺ 460.32 | 1H NMR (400 MHz, DMSO) δ 8.55 (1H, d), 8.35 (1H, d), 7.75 (1H, d), 7.3 (1H, t), 7.1 (1H, d), 7.05 (1H, d), 3.65 (1H, m), 3.55 (2H, d), 2.45 (3H, s), 1.9 (2H, m), 1.7 (3H, m), 1.4 (6H, s), 1.15 (4H, m). |
| 2.26 | 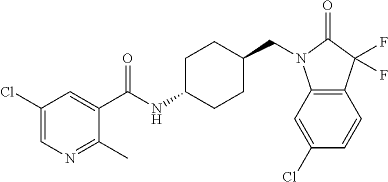 | Trans-5-Chloro-N-[4-(6- chloro-3,3-difluoro-2- oxo-2,3-dihydro-indol- 1-ylmethyl)-cyclohexyl]-2- methyl-nicotinamide | Rt = 2.6 min; [M + H]⁺ 468.23 | 1H NMR (400 MHz, DMSO) δ 8.55 (1H, d), 8.4 (1H, d), 7.8 (1H, d), 7.75 (1H, d), 7.55 (1H, s), 7.3 (1H, d), 3.7 (1H, m), 3.55 (2H, d), 2.45 (3H, s), 1.9 (2H, m), 1.7 (3H, m), 1.15 (4H, m). |
| 2.27 | 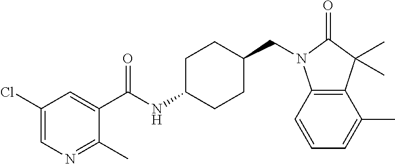 | Trans-5-Chloro-2- methyl-N-[4-(3,3,4- trimethyl-2-oxo-2,3- dihydro-indol-1- ylmethyl)-cyclohexyl]- nicotinamide | Rt = 2.62 min; [M + H]⁺ 440.35 | 1H NMR (400 MHz, DMSO) δ 8.55 (1H, d), 8.35 (1H, d), 7.8 (1H, d), 7.25 (1H, t), 6.95 (1H, d), 6.8 (1H, d), 3.65 (1H, m), 3.55 (2H, d), 2.45 (3H, s), 2.35 (3H, s), 1.9 (2H, m), 1.7 (3H, m), 1.35 (6H, s), 1.15 (4H, m). |
| 2.28 | 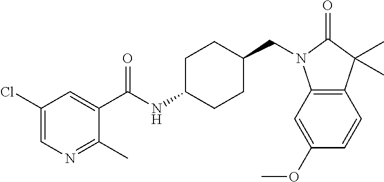 | Trans-5-Chloro-N-[4-(6- methoxy-3,3-dimethyl-2- oxo-2,3-dihydro-indol- 1-ylmethyl)-cyclohexyl]-2- methyl-nicotinamide | Rt = 0.88 min; [M + H]⁺ 456 Method 2minLC_30_v002 | 1H NMR (400 MHz, CDCl3) δ 8.50 (1H, s), 7.65 (1H, s), 7.10 (1H, d), 6.60 (1H, d), 6.45 (1H, s), 5.65 (1H, d), 3.95 (1H, m), 3.85 (3H, s), 3.55 (2H, d), 2.65 (3H, s), 2.15 (2H, m), 1.80 (3H, m), 1.35 (6H, s), 1.25 (4H, m). |
| 2.29 | 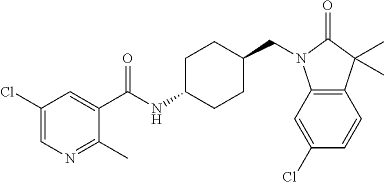 | Trans-5-Chloro-N-[4-(6- chloro-3,3-dimethyl-2- oxo-2,3-dihydro-indol- 1-ylmethyl)-cyclohexyl]-2- methyl-nicotinamide | Rt = 1 min; [M + H]⁺ 460.1 Method 2minLC_30_v002 | 1H NMR (400 MHz, CDCl3) δ 8.50 (1H, s), 7.65 (1H, s), 7.15 (1H, d), 7.05 (1H, d), 6.85 (1H, s), 5.65 (1H, d), 3.95 (1H, m), 3.55 (2H, d), 2.65 (3H, m), 2.15 (2H, m), 1.80 (3H, m), 1.40 (6H, s), 1.25 (4H, m). |
| 2.30 | 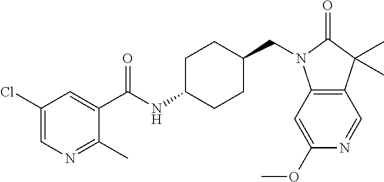 | Trans-5-Chloro-N-[4-(6- methoxy-3,3-dimethyl-2- oxo-2,3-dihydro- pyrrolo[3,2-c]pyridin- 1-ylmethyl)-cyclohexyl]-2- methyl-nicotinamide | Rt = 0.87 min; [M + H]⁺ 457.4 Method 2minLC_30_v002 | 1H NMR (400 MHz, CDCl3) δ 8.50 (1H, s), 7.90 (1H, s), 7.60 (1H, s), 6.20 (1H, s), 5.55 (1H, d), 4.0 (4H, m), 3.50 (2H, d), 2.60 (3H, s), 2.15 (2H, m), 1.80 (3H, m), 1.40 (6H, s), 1.25 (4H, m). |
| 2.31 | 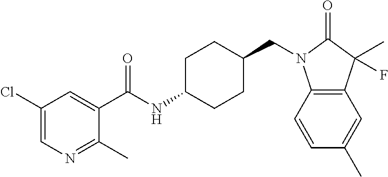 | Trans-5-Chloro-N-[4-(-3- fluoro-3,5-dimethyl-2- oxo-2,3-dihydro-indol-1- ylmethyl)-cyclohexyl]-2- methyl-nicotinamide | Rt = 2.54 min; [M − F]⁺ 424.4 | 1H NMR (400 MHz, DMSO) δ 8.53 (1H, d), 8.37 (1H, d), 7.78 (1H, d), 7.39 (1H, s), 7.23 (1H, d), 7.06 (1H, d), 3.6- 3.76 (1H, m), 3.51 (2H, d), 2.46 (3H, s), 2.30 (3H, s), 1.85-1.98 (2H, m), 1.6-1.8 (6H, m), 1.05-1.3 (4H, m) |
| 2.32 | 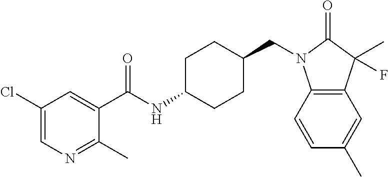 | Trans-5-Chloro-N-[4-(-3- fluoro-3,5-dimethyl-2- oxo-2,3-dihydro-indol-1- ylmethyl)-cyclohexyl]-2- methyl-nicotinamide | Rt = 2.53 min; [M − F]⁺ 424.4 | 1H NMR (400 MHz, DMSO) δ 8.53 (1H, d), 8.37 (1H, d), 7.78 (1H, d), 7.39 (1H, s), 7.23 (1H, d), 7.06 (1H, d), 3.6- 3.8 (3H, m), 2.46 (3H, s), 2.30 (3H, s), 1.82-1.98 (2H, m), 1.58-1.8 (6H, m), 1.04-1.3 (4H, m) |
| 2.33 | 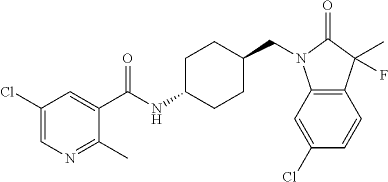 | Trans-5-Chloro-N-[4-(-6- chloro-3-fluoro-3- methyl-2-oxo-2,3- dihydro-indol-1- ylmethyl)-cyclohexyl]- 2-methyl-nicotinamide | Rt = 2.56 min; [M − F]⁺ 444.3 | 1H NMR (400 MHz, DMSO) δ 8.53 (1H, d), 8.38 (1H, d), 7.78 (1H, d), 7.60 (1H, dd), 7.38 (1H, s), 7.18 (1H, dd), 3.61-3.77 (1H, m), 3.54 (2H, d), 2.46 (3H, s), 1.82-98 (2H, m), 1.6-1.8 (6H, m), 1.04-1.3 (4H, m) |
| 2.34 | 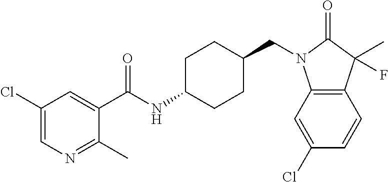 | Trans-5-Chloro-N-[4-(-6- chloro-3-fluoro-3- methyl-2-oxo-2,3- dihydro-indol-1- ylmethyl)-cyclohexyl]- 2-methyl-nicotinamide | Rt = 2.56 min; [M − F]⁺ 444.3 | 1H NMR (400 MHz, DMSO) δ 8.53 (1H, d), 8.38 (1H, d), 7.78 (1H, d), 7.60 (1H, dd), 7.38 (1H, s), 7.18 (1H, dd), 3.61-3.77 (1H, m), 3.54 (2H, d), 2.46 (3H, s), 1.84-98 (2H, m), 1.6-1.8 (6H, m), 1.05-1.3 (4H, m) |
| 2.35 | 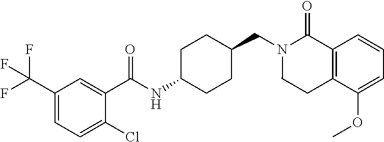 | Trans-2-Chloro-N-[4-(5- methoxy-1-oxo-3,4- dihydro-1H-isoquinolin- 2-ylmethyl)- cyclohexyl]-5- trifluoromethyl- benzamide | Rt = 1.33 min; [M + H]⁺ 495.3 Method 2minLC_30_v002. | 1H NMR (400 MHz, CDCl3) δ 7.99 (1H, d), 7.68 (1H, d), 7.61 (1H, dd), 7.53 (1H, d), 7.31 (1H, t), 7.00 (1H, d), 6.04 (1H, d), 4.00 (1H, m), 3.87 (3H, s), 3.57 (2H, t), 3.44 (2H, d), 2.99 (2H, t), 2.19 (2H, m), 1.90 (2H, m), 1.83 (1H, m), 1.25 (4H, m). |
| 2.36 | 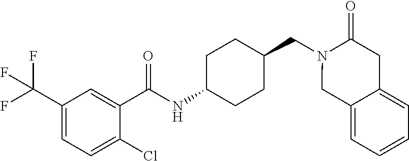 | Trans-2-Chloro-N-[4-(3- oxy-3,4-dihydro-1H- isoquinolin-2-ylmethyl)- cyclohexyl]-5- trifluoromethyl- benzamide | Rt = 1.28 min; [M + H]⁺ 465.3 Method 2minLC_30_v002 | 1H NMR (400 MHz, CDCl3) δ 7.9 (1H, s), 7.6 (1H, d), 7.55 (1H, d), 7.25 (3H, m), 7.2 (2H, m), 5.95 (1H, br), 4.5 (2H, s), 3.95 (1H, m), 3.65 (2H, s), 3.4 (2H, d), 2.15 (2H, br), 1.8 (3H, br), 1.2 (4H, m). |
| 2.37 | 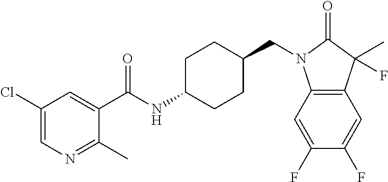 | Trans-5-Chloro-2- methyl-N-[4-(-3,5,6- trifluoro-3-methyl-2- oxo-2,3-dihydro- indol-1-ylmethyl)- cyclohexyl]- nicotinamide | Rt = 2.49 min; [M − F]⁺ 446.4 | 1H NMR (400 MHz, DMSO) δ 8.53 (1H, d), 8.38 (1H, d), 7.86 (1H, app t), 7.78 (1H, d), 7.48 (1H, dd), 3.6-3.77 (1H, m), 3.51 (2H, d), 2.46 (3H, s), 1.82-1.97 (2H, m), 1.6-1.8 (6H, m), 1.03-1.32 (4H, m). |
| 2.38 | 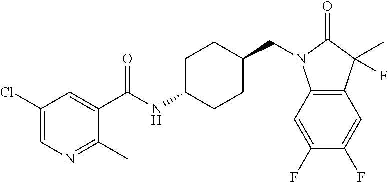 | Trans-5-Chloro-2- methyl-N-[4-(-3,5,6- trifluoro-3-methyl-2- oxo-2,3-dihydro- indol-1-ylmethyl)- cyclohexyl]- nicotinamide | Rt = 2.49 min; [M − F]⁺ 446.3 | 1H NMR (400 MHz, DMSO) δ 8.53 (1H, d), 8.38 (1H, d), 7.86 (1H, app t), 7.78 (1H, d), 7.48 (1H, dd), 3.6-3.75 (1H, m), 3.51 (2H, d), 2.46 (3H, s), 1.84-1.96 (2H, m), 1.6-1.8 (6H, m), 1.03-1.3 (4H, m). |
| 2.39 | 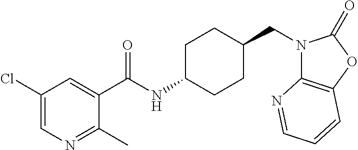 | Trans-5-Chloro-2- methyl-N-[4-(2-oxo- oxazolo[4,5-b]pyridin-3- ylmethyl)-cyclohexyl]- nicotinamide | Rt = 2.37 min; [M + H]⁺ 401.31 | 1H NMR (400 MHz, DMSO) δ 8.52 (1H, d), 8.39 (1H, d), 8.12 (1H, q), 7.79 (1H, d), 7.71 (1H, q), 7.19 (1H, q), 3.70 (3H, m), 2.47 (3H, s), 1.90 (3H, m), 1.72 (2H, m), 1.20 (4H, m). |
| 2.40 | 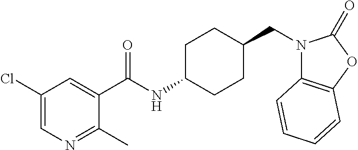 | Trans-5-Chloro-2- methyl-N-[4-(2-oxo- benzooxazol-3- ylmethyl)-cyclohexyl]- nicotinamide | Rt = 2.47 min; [M + H]⁺ 400.34 | 1H NMR (400 MHz, DMSO) δ 8.52 (1H, d), 8.39 (1H, d), 7.79 (1H, d), 7.32 (2H, d), 7.21 (1H, t), 7.12 (1H, t), 3.70 (3H, m), 2.48 (3H, s), 1.90 (2H, m), 1.80 (1H, m), 1.71 (2H, m), 1.20 (4H, m). |
| 2.41 | 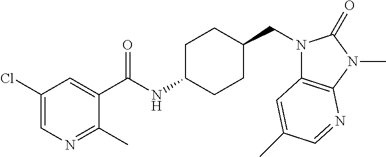 | Trans-5-Chloro-N-[4- (3,6-dimethyl-2-oxo-2,3- dihydro-imidazo[4,5- b]pyridin-1-ylmethyl)- cyclohexyl]-2-methyl- nicotinamide | Rt = 2.41 min; [M + H]⁺ 428.54 | 1H NMR (400 MHz, DMSO) δ 8.52 (1H, d), 8.39 (1H, d), 7.81 (1H, s), 7.79 (1H, d), 7.42 (1H, s), 3.67 (3H, m), 3.31 (3H, s), 2.48 (3H, s), 2.31 (3H, s), 1.90 (2H, m), 1.74 (1H, m), 1.67 (2H, m), 1.18 (4H, m). |
| 2.42 | 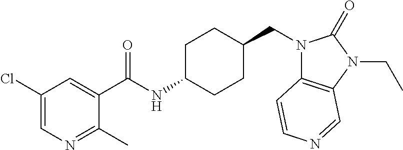 | Trans-5-Chloro-N-[4-(3- ethyl-2-oxo-2,3-dihydro- imidazo[4,5-c]pyridin-1- ylmethyl)-cyclohexyl]-2- methyl-nicotinamide | Rt = 1.85 min; [M + H]⁺ 428.33 | 1H NMR (400 MHz, DMSO) δ 8.89 (1H, br), 8.59 (1H, br), 8.52 (1H, d), 8.40 (1H, d), 7.90 (1H, br), 7.79 (1H, d), 4.01 (2H, q), 3.88 (2H, d), 3.69 (1H, m), 2.48 (3H, s), 1.90 (2H, m), 1.79 (1H, m), 1.69 (2H, m), 1.29 (3H, t), 1.20 (4H, m). |
| 2.43 | 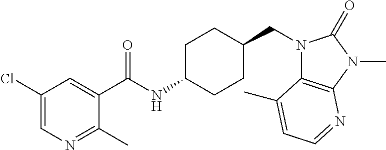 | Trans-5-Chloro-N-[4- (3,7-dimethyl-2-oxo-2,3- dihydro-imidazo[4,5- b]pyridin-1-ylmethyl)- cyclohexyl]-2-methyl- nicotinamide | Rt = 2.38 min; [M + H]⁺ 428.32 | 1H NMR (400 MHz, DMSO) δ 8.52 (1H, s), 8.39 (1H, d), 7.89 (1H, d), 7.79 (1H, s), 6.89 (1H, d), 3.85 (2H, d), 3.70 (1H, m), 3.31 (3H, s), 2.51 (3H, s), 2.48 (3H, s), 1.90 (2H, m), 1.68 (3H, m), 1.20 (4H, m). |
| 22.44 | 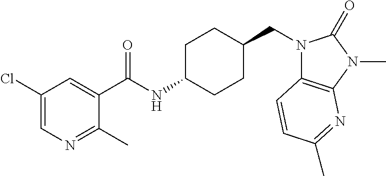 | Trans-5-Chloro-N-[4- (3,5-dimethyl-2-oxo-2,3- dihydro-imidazo[4,5- b]pyridin-1-ylmethyl)- cyclohexyl]-2-methyl- nicotinamide | Rt = 2.41 min; [M + H]⁺ 428.33 | 1H NMR (400 MHz, DMSO) δ 8.52 (1H, s), 8.39 (1H, d), 7.79 (1H, d), 7.45 (1H, d), 6.91 (1H, d), 3.69 (3H, m), 3.31 (3H, s), 2.47 (3H, s), 2.42 (3H, s), 1.89 (2H, m), 1.73 (1H, m), 1.68 (2H, m), 1.19 (4H, m). |
| 2.45 | 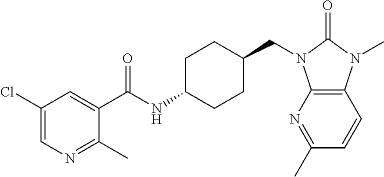 | Trans-5-Chloro-N-[4- (1,5-dimethyl-2-oxo-1,2- dihydro-imidazo[4,5- b]pyridin-3-ylmethyl)- cyclohexyl]-2-methyl- nicotinamide | Rt = 2.43 min; [M + H]⁺ 428.27 | 1H NMR (400 MHz, DMSO) δ 8.53 (1H, s), 8.37 (1H, d), 7.78 (1H, s), 7.38 (1H, d), 6.93 (1H, d), 3.70 (3H, m), 3.33 (3H, s), 2.47 (3H, s), 2.45 (3H, s), 1.86 (3H, m), 1.66 (2H, m), 1.17 (4H, m). |
| 2.46 | 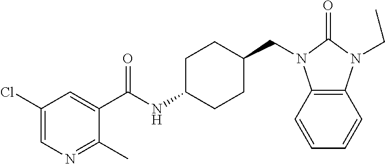 | Trans-5-Chloro-N-[4-(3- ethyl-2-oxo-2,3-dihydro- benzoimidazol-1- ylmethyl)-cyclohexyl]-2- methyl-nicotinamide | Rt = 2.53 min; [M + H]⁺ 427.25 | 1H NMR (400 MHz, DMSO) δ 8.53 (1H, s), 8.38 (1H, d), 7.78 (1H, s), 7.21 (2H, m), 7.06 (2H, m), 3.88 (2H, m), 3.70 (3H, m), 2.46 (3H, s), 1.88 (2H, m), 1.76 (1H, m), 1.67 (2H, m), 1.20 (7H, m). |
| 2.47 | 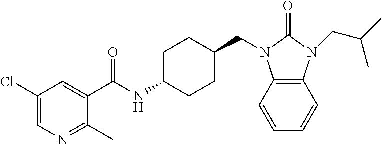 | Trans-5-Chloro-N-[4-(3- isobutyl-2-oxo-2,3- dihydro-benzoimidazol- 1-ylmethyl)-cyclohexyl]- 2-methyl-nicotinamide | Rt = 2.64 min; [M + H]⁺ 455.33 | 1H NMR (400 MHz, DMSO) δ 8.53 (1H, d), 8.37 (1H, d), 7.78 (1H, s), 7.20 (2H, m), 7.05 (2H, m), 3.67 (5H, m), 2.46 (3H, s), 2.11 (1H, m), 1.88 (2H, m), 1.78 (1H, m), 1.67 (2H, m), 1.17 (4H, m), 0.88 (6H, d). |
| 2.48 | 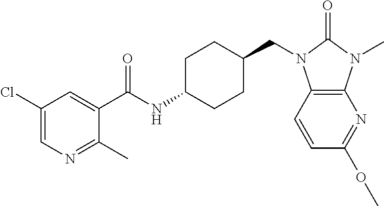 | Trans-5-Chloro-N-[4-(5- methoxy-3-methyl-2- oxo-2,3-dihydro- imidazo[4,5-b]pyridin-1- ylmethyl)-cyclohexyl]-2- methyl-nicotinamide | Rt = 2.48 min; [M + H]⁺ 444.27 | 1H NMR (400 MHz, DMSO) δ 8.53 (1H, s), 8.38 (1H, d), 7.78 (1H, s), 7.56 (1H, d), 6.48 (1H, d), 3.86 (3H, s), 3.67 (3H, m), 3.31 (3H, s, N—CH3 assumed under water signal), 2.46 (3H, s), 1.89 (2H, m), 1.73 (1H, m), 1.66 (2H, m), 1.17 (4H, m). |
| 2.49 | 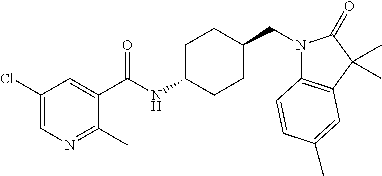 | Trans-5-Chloro-2- methyl-N-[4-(3,3,5- trimethyl-2-oxo-2,3- dihydro-indol-1- ylmethyl)-cyclohexyl]- nicotinamide | Rt = 2.64 min; [M + H]⁺ 440.41 | 1H NMR (400 MHz, DMSO) δ 8.52 (1H, s), 8.39 (1H, d), 8.39 (1H, d), 7.78 (1H, s), 7.18 (1H, s), 7.03 (1H, d), 6.96 (1H, d), 3.68 (1H, m), 3.51 (2H, d), 2.48 (3H, s), 2.30 (3H, s), 1.90 (2H, m), 1.71 (1H, m), 1.68 (2H, m), 1.29 (6H, s), 1.14 (4H, m). |
| 2.50 | 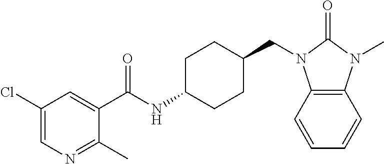 | Trans-5-Chloro-2- methyl-N-[4-(3-methyl- 2-oxo-2,3-dihydro- benzoimidazol-1- ylmethyl)-cyclohexyl]- nicotinamide | Rt = 2.47 min; [M + H]⁺ 413.33 | 1H NMR (400 MHz, DMSO) δ 8.53 (1H, s), 8.38 (1H, d), 7.78 (1H, s), 7.21 (1H, m), 7.14 (1H, m), 7.06 (2H, m), 3.68 (3H, m), 3.32 (3H, s), 2.46 (3H, s), 1.89 (2H, m), 1.76 (1H, m), 1.67 (2H, m), 1.18 (4H, m). |
| 2.51 | 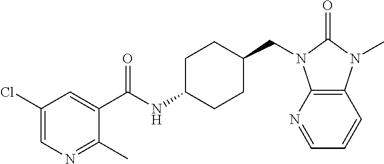 | Trans-5-Chloro-2- methyl-N-[4-(1-methyl- 2-oxo-1,2-dihydro- imidazo[4,5-b]pyridin-3- ylmethyl)-cyclohexyl]- nicotinamide | Rt = 2.5 min; [M + H]⁺ 414.4 | 1H NMR (400 MHz, DMSO) δ 8.54 (1H, s), .8.38 (1H, d), 7.99 (1H, d), 7.79 (1H, s), 7.50 (1H, d), 7.08 (1H, t), 3.74 (2H, d), 3.70 (1H, m), 3.36 (3H, s), 2.47 (3H, s) 1.88 (3H, m), 1.67 (2H, m), 1.17 (4H, m). |
| 2.52 | 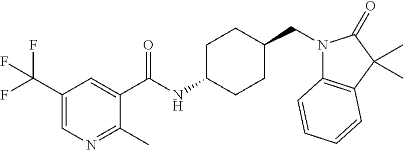 | Trans-N-[4-(3,3- Dimethyl-2-oxo-2,3- dihydro-indol-1- ylmethyl)-cyclohexyl]- 2-methyl-5- trifluoromethyl- nicotinamide | Rt = 2.64 min; [M + H]⁺ 460.38 | 1H NMR (400 MHz, CDCl3) δ 8.90 (1H, d), 8.48 (1H, d), 8.01 (1H, d), 7.33 (1H, m), 7.27 (1H, m), 7.08 (2H, m), 3.70 (1H, m), 3.52 (2H, d), 2.59 (3H, s), 1.92 (2H, m), 1.72 (1H, m), 1.69 (2H, m), 1.30 (6H, s), 1.17 (4H, m). |
| 2.53 | 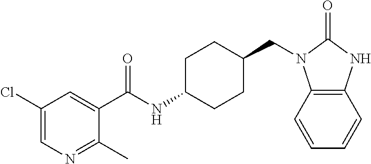 | Trans-5-Chloro-2- methyl-N-[4-(2-oxo-2,3- dihydro-benzoimidazol- 1-ylmethyl)-cyclohexyl]- nicotinamide | Rt = 2.43 min; [M + H]⁺ 399.91 | 1H NMR (400 MHz, DMSO) δ 10.81 (1H, s), 8.53 (1H, s), 8.39 (1H, d), 7.79 (1H, s), 7.15 (1H, d), 6.99 (3H, m), 3.68 (3H, m), 2.47 (3H, s), 1.90 (2H, m), 1.75 (1H, m), 1.67 (2H, m), 1.19 (4H, m). |
| 2.54 | 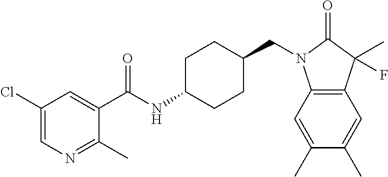 | Enantiomer 1 of Trans-5- Chloro-N-[4-(3-fluoro- 3,5,6-trimethyl-2-oxo- 2,3-dihydro-indol-1- ylmethyl)-cyclohexyl]-2- methyl-nicotinamide | Rt = 2.52 min; [M − F+]438.38 | 1H NMR (400 MHz, CDCl₃) 8.47 (1H, s), 7.74 (1H, s), 7.20 (1H, s), 6.64 (1H, s), 5.93 (1H, bs), 3.85- 4.01 (1H, m), 3.44- 3.63 (2H, m), 2.68 (3H, s), 2.32 (3H, s), 2.27 (3H, s), 2.08- 2.19 (2H, m), 1.75- 1.91 (3H, m), 1.59 (3H, s), 1.15-1.37 (4H, m) |
| 2.55 | 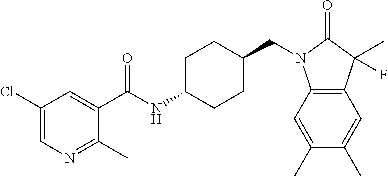 | Enantiomer 2 of Trans-5- Chloro-N-[4-(3-fluoro- 3,5,6-trimethyl-2-oxo- 2,3-dihydro-indol-1- ylmethyl)-cyclohexyl]-2- methyl-nicotinamide | Rt = 2.52 min; [M − F+]438.36 | 1H NMR (400 MHz, CDCl₃) 8.48 (1H, s), 7.67 (1H, s), 7.28 (1H, s), 6.64 (1H, s), 5.74 (1H, bs), 3.87- 4.01 (1H, m), 3.45- 3.63 (2H, m), 2.64 (3H, s), 2.32 (3H, s), 2.27 (3H, s), 2.08- 2.19 (2H, m), 1.75- 1.91 (3H, m), 1.59 (3H, s), 1.14-1.36 (4H, m) |
| 2.56 | 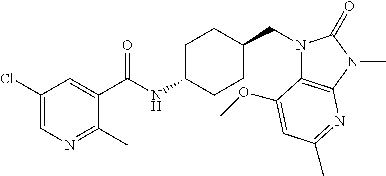 | Trans-5-Chloro-N-[4-(7- methoxy-3,5-dimethyl-2- oxo-2,3-dihydro- imidazo[4,5-b]pyridin-1- ylmethyl)-cyclohexyl]-2- methyl-nicotinamide | Rt = 2.52 min; [M + H]⁺ 458.44 | 1H NMR (400 MHz, DMSO) δ 8.52 (1H, s), 8.38 (1H, d), 7.79 (1H, s), 6.82 (1H, s), 3.95 (3H, s), 3.78 (2H, d), 3.69 (1H, m), 3.31 (3H, s), 2.47 (3H, s), 2.40 (3H, s), 1.90 (2H, m), 1.70 (1H, m), 1.61 (2H, m), 1.18 (4H, m). |
| 2.57 | 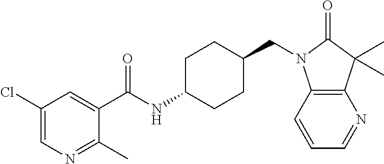 | Trans-5-Chloro-N-[4- (3,3-dimethyl-2-oxo-2,3- dihydro-pyrrolo[3,2- b]pyridin-1-ylmethyl)- cyclohexyl]-2-methyl- nicotinamide | Rt = 2.33 min; [M + H]⁺ 427.39 | 1H NMR (400 MHz, DMSO) δ 8.55 (1H, d), 8.40 (1H, d), 8.21 (1H, d), 7.81 (1H, d), 7.64 (1H, d), 7.39 (1H, m), 3.68 (1H, m), 3.59 (2H, d), 2.47 (3H, s), 1.89 (2H, m), 1.70 (3H, m), 1.34 (6H, s), 1.17 (4H, m). |
| 2.58 | 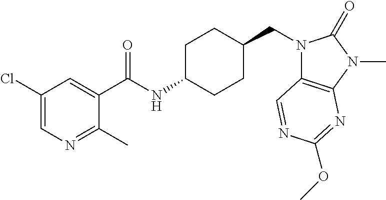 | Trans-5-Chloro-N-[4-(2- methoxy-9-methyl-8- oxo-8,9-dihydro-purin-7- ylmethyl)-cyclohexyl]-2- methyl-nicotinamide | Rt = 2.29 min; [M + H]⁺ 445.35 | 1H NMR (400 MHz, DMSO) δ 8.53 (1H, s), 8.39 (1H, d), 8.21 (1H, s), 7.80 (1H, d), 3.90 (3H, 6), 3.69 (3H, m), 3.30 (3H, s), 2.48 (3H, s), 1.90 (2H, m), 1.75 (1H, m), 1.69 (2H, m), 1.19 (4H, m). |
| 2.59 | 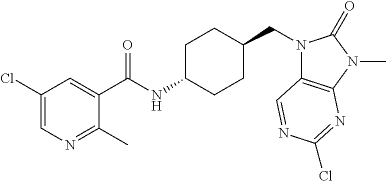 | Trans-5-Chloro-N-[4-(2- chloro-9-methyl-8-oxo- 8,9-dihydro-purin-7- ylmethyl)-cyclohexyl]-2- methyl-nicotinamide | Rt = 2.37 min; [M + H]⁺ 449.29 | 1H NMR (400 MHz, DMSO) δ 8.52 (1H, d), 8.41 (1H, s), 8.39 (1H, d), 7.79 (1H, d), 3.72 (2H, d), 3.69 (1H, m), 2.51 (3H, s), 2.47 (3H, s), 1.89 (2H, m), 1.78 (1H, m), 1.71 (2H, m), 1.19 (4H, m). |

*Prepared from tert-butyl 2-oxospiro[indoline-3,4′-piperidine]-1′-carboxylate followed by deprotection with TFA/DCM to afford Ex.2.21

### Example 3.1

### Trans-2-Chloro-N-[4-(5-chloro-2-oxo-2,3-dihydro-indol-1-ylmethyl)-cyclohexyl]-5-trifluoromethyl-benzamide

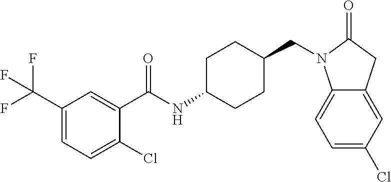

To a stirring solution of 5-chloro-2-oxindole (commercially available) (35.8 mg, 0.214 mmol) in DMF (2 ml) was added NaH (8.55 mg, 0.214 mmol). The mixture was stirred for 1.5 hours at RT and then treated with trifluoro-methanesulfonic acid 4-(2-chloro-5-trifluoromethyl-benzoylamino)-cyclohexylmethylester (Intermediate E) (50 mg, 0.107 mmol). After stirring at RT for 1 hour, the mixture was diluted with water and extracted with EtOAc (3×20 ml). The combined organic extracts were washed with water, brine, dried (MgSO₄) and concentrated in vacuo to yield an orange oil. Purification of the oil by preparative LC-MS afforded the title compound; LC-MS Rt 1.36 mins; MS m/z 485.2 [M+H]⁺; Method 2minLC_{—}30_v002. ¹H NMR (400 MHz, CDCl3) δ 7.90 (1H, s), 7.61 (1H, dd), 7.53 (1H, d), 7.26 (2H, m), 6.77 (1H, d), 5.99 (1H, d), 4.00 (1H, m), 3.56 (4H, m), 2.19 (2H, m), 1.82 (3H, m), 1.25 (2H, m).

The compounds of the following tabulated Examples (Table 3) were prepared by a similar method to that of Example 3.1 using the appropriate triflate and oxindole starting compounds, the preparations of which are described hereinafter (see ‘Intermediates’ section).

TABLE 3

| | | | Rention Time (min), | |
|---|---|---|---|---|
| | | | [M + H]⁺ (Method | |
| | | | LowpH_v002) (Unless | |
| Ex. | Structure | Name | otherwise specified) | ¹H NMR |
| 3.2 | 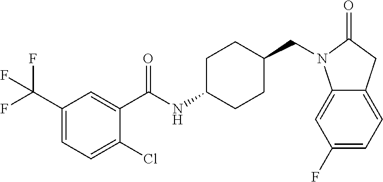 | Trans-2-Chloro-N- [4-(6-fluoro-2-oxo- 2,3-dihydro- indol-1- ylmethyl)- cyclohexyl]-5- trifluoromethyl- bcnzamide | Rt 1.32 min [M + H]⁺ 469.2 (Method 2 min LC_30_v002) | (400 MHz, CDCl3) δ 7.90 (1H, d), 7.61 (1H, dd), 7.53 (1H, d), 7.20 (1H, dd), 6.74 (1H, m), 6.58 (1H, dd), 6.02 (1H, d), 4.00 (1H, m), 3.54 (4H, m), 2.20 (2H, m), 1.83 (3H, m), 1.27 (4H, m). |
| 3.3 | 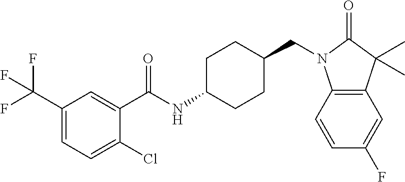 | Trans-2-Chloro-N- [4-(5-fluoro-3,3- dimethyl-2-oxo-2,3- dihydro-indol-1- ylmethyl)- cyclohexyl]-5- trifluoromethyl- benzamide | Rt 1.18 min [M + H]⁺ 497.4 (Method 2 min LC_30_v002) | (400 MHz, CDCl3) δ 7.9 (1H, s), 7.6 (1H, d), 7.5 (1H, d), 6.95 (2H, m), 6.75 (1H, m), 6.1 (1H, d), 3.95 (1H, m), 3.55 (2H, d), 2.15 (2H, m), 1.8 (3H, m), 1.4 (6H, s), 1.35 (4H, m). |
| 3.4 | 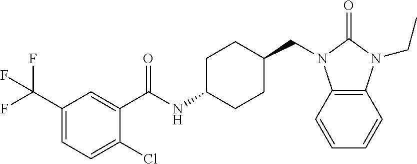 | Trans-2-Chloro-N- [4-(3-ethyl-2-oxo- 2,3-dihydro- bcnzoimidazol-1- ylmethyl)- cyclohexyl]-5- trifluoromethyl- benzamide | Rt 2.56 min [M + H]⁺ 480.36 | (400 MHz, DMSO) δ 8.48 (1H, d), 7.81 (1H, m), 7.72 (2H, m), 7.22 (2H, m), 7.05 (2H, m), 3.88 (2H, q), 3.69 (3H, m), 1.90 (2H, m), 1.74 (1H, m), 1.65 (2H, m), 1.23 (7H, m). |
| 3.5 | 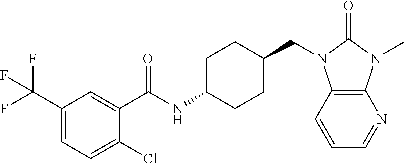 | Trans-2-Chloro-N- [4-(3-methyl-2- oxo-2,3-dihydro- imidazo[4,5- b]pyridin-1- ylmethyl)- cyclohexyl]-5- trifluoromethyl- benzamide | Rt 2.47 min [M + H]⁺ 467.32 | (400 MHz, DMSO) δ 8.49 (1H, d), 7.98 (1H, d), 7.81 (1H, m), 7.62 (2H, m), 7.55 (1H, d), 7.07 (1H, m), 3.72 (2H, d), 1.91 (2H, m), 1.75 (1H, m), 1.68 (2H, m), 1.18 (4H, m). |
| 3.6 | 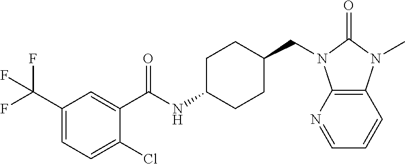 | Trans-2-Chloro-N- [4-(1-methyl-2- oxo-1,2-dihydro- imidazo[4,5- b]pyridin-3- ylmethyl)- cyclohexyl]-5- trifluoromethyl- benzamide | Rt 2.47 min [M + H]⁺ 467.32 | (400 MHz, DMSO) δ 8.46 (1H, d), 7.98 (1H, m), 7.80 (1H, m), 7.73 (2H, m), 7.49 (1H, m), 7.08 (1H, m), 3.73 (2H, d), 3.70 (1H, m), 3.35 (3H, s), 1.91 (2H, m), 1.86 (1H, m), 1.67 (2H, m), 1.17 (4H, m) |
| 3.7 * | 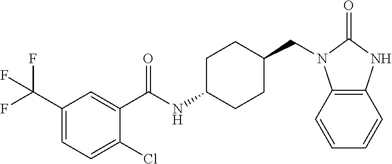 | Trans-2-Chloro-N- [4-(2-oxo-2,3- dihydro- bcnzoimidazol-1- ylmethyl)- cyclohexyl]-5- trifluoromethyl- benzamide | Rt 2.51 min [M + H]⁺ 452.3 | (400 MHz, DMSO) δ 10.80 (1H, s), 8.47 (1H, d), 7.79 (1H, m), 7.73 (2H, m), 7.14 (1H, m), 6.99 (3H, m), 3.70 (1H, m), 3.65 (2H, d), 1.91 (2H, m), 1.75 (1H, m), 1.67 (2H, m), 1.18 (4H, m). |
| 3.8 | 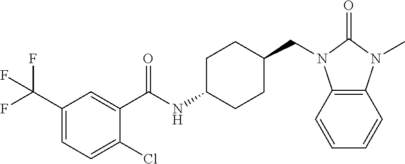 | Trans-2-Chloro-N- [4-(3-methyl-2- oxo-2,3-dihydro- benzoimidazol-1- ylmethyl)- cyclohexyl]-5- trifluoromethyl- benzamide | Rt 2.55 min [M + H]⁺ 466.54 | (400 MHz, DMSO) δ 8.48 (1H, d), 7.80 (1H, d), 7.73 (2H, d), 7.20 (1H, m), 7.14 (1H, m), 7.05 (2H, m), 3.32 (1H, s), 1.89 (4H, m), 1.77 (1H, m), 1.68 (4H, m). |

* This compound was prepared from trans-trifluoromethanesulfonic acid 4-(2-chloro-5-trifluoromethyl-benzoylamino)-cyclohexylmethylester (Intermediate E) and 2-Oxo-2,3-dihydro-benzoimidazole-1-carboxylic acid tert-butyl ester (Intermediate RF). Subsequent deprotection with 4M HCl/Dioxan/MeOH affords the final compound.

### Example 4.1

### Trans-4-Fluoro-N-[4-(3-methyl-2-oxo-2,3-dihydro-benzoimidazol-1-ylmethyl)-cyclohexyl]-3-trifluoromethyl-benzamide trifluoroacetate

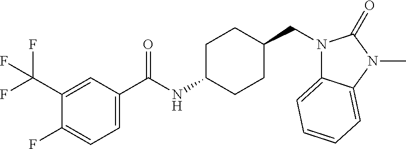

A solution of 4-fluoro-3-trifluoromethyl-benzoic acid (0.101 mmol) in DMF (1 ml) was treated with DIPEA (81 μl, 0.461 mmol) followed by HATU (52.8 mg. 0.139 mmol) and stirred at RT for 15 minutes. This mixture was added to a solution of 1-(4-amino-cyclohexylmethyl)-3-methyl-1,3-dihydro-benzo imidazol-2-one hydrochloride (Intermediate RR) (150 mg, 0.101 mmol) in DMF (5 ml) and stirred at RT for 2 hours. The solvent was removed in vacuo the resulting residue was dissolved in DCM (2 ml) and washed with water (2 ml). The organic portion was passed through a phase separator column and concentrated in vacuo. The residue was dissolved in DMSO and purification by preparative LC-MS eluting with MeCN (0.1% TFA) in water (0.1% TFA) afforded the title product; LC-MS Rt 1.22 mins; MS m/z 450.3 [M+H]+; Method 2minLC_v003

### Example 4.2

### Trans-2,5-Dichloro-N-[4-(3-methyl-2-oxo-2,3-dihydro-benzoimidazol-1-ylmethyl)-cyclohexyl]-benzamide trifluoroacetate

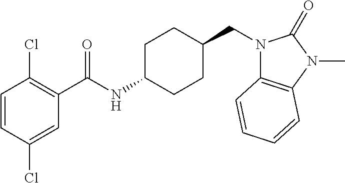

This compound was prepared analogously to Example 4.1 by replacing 4-fluoro-3-trifluoromethyl-benzoic acid with the appropriate acid; LC-MS Rt 1.18 mins; MS m/z 432.2 [M+H]+; Method 2minLC_v003.

### Example 5.1

### Trans-N-[4-(3,3-Dimethyl-2-oxo-2,3-dihydro-indol-1-ylmethyl)-cyclohexyl]-4-fluoro-3-trifluoromethyl-benzamide

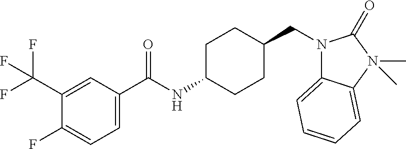

A suspension of 4-fluoro-3-trifluoromethyl-benzoyl chloride (33.3 mg, 0.147 mmol) in DCM (367 μl) was treated with a solution of 1-(4-amino-cyclohexylmethyl)-3,3-dimethyl-1,3-dihydro-indol-2-one (Intermediate RQ) (20 mg, 0.073 mmol) in DMF (467 μl) and pyridine (29.7 μl). The reaction mixture was shaken at RT overnight. The mixture was filtered and purification by preparative LC-MS eluting with MeCN (0.1% TFA) in water (0.1% TFA) afforded the title product; LC-MS Rt 1.31 mins; MS m/z 463.3 [M+H]+; Method 2minLC_v003.

### Example 5.2

### Trans-2,5-Dichloro-N-[4-(3,3-dimethyl-2-oxo-2,3-dihydro-indol-1-ylmethyl)-cyclohexyl]-benzamide

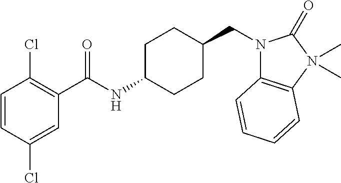

This compound was prepared analogously to Example 5.1 by replacing 4-fluoro-3-trifluoromethyl-benzoyl chloride with the appropriate acid chloride; LC-MS Rt 1.28 mins; 445.2 [M+H]+; Method 2minLC_v003.

### Example 5.3

### Trans-N-[4-(3,3-Dimethyl-2-oxo-2,3-dihydro-indol-1-ylmethyl)-cyclohexyl]-3-methoxy-benzamide

This compound was prepared analogously to Example 5.1 by replacing 4-fluoro-3-trifluoromethyl-benzoyl chloride with the appropriate acid chloride; LC-MS Rt 1.2 mins; 407.3 [M+H]+; Method 2minLC_v003.

### PREPARATION OF INTERMEDIATES

### Intermediate A

### Trans-toluene-4-sulfonic acid 4-[(5-chloro-2-methyl-pyridine-3-carbonyl)-amino]-cyclohexylmethyl ester

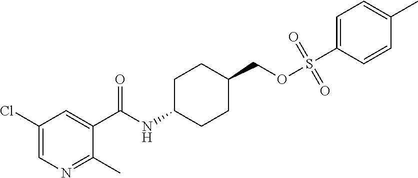

### Step 1: 5-Chloro-2-methylnicotinoyl chloride

5-Chloro-2-methyl-nicotinic acid (4.15 g, 24.2 mmol) was placed in a flask with DCM (100 ml) and oxalyl chloride (3.68 g, 29 mmol). DMF (200 μl) was added and the reaction mixture was stirred at RT for 1 hour (gas evolution). The mixture was filtered and the solvent was removed in vacuo to afford the title product which was used in the next step without further purification.

### Step 2: Trans-4-[(5-Chloro-2-methyl-pyridine-3-carbonyl)-amino]-cyclohexanecarboxylic acid methyl ester

Trans-4-amino-cyclohexanecarboxylic acid methyl ester (commercially available) (2.14 g, 11.05 mmol) was suspended in THF (50 ml) and Et₃N (2.79 g, 27.6 mmol) and cooled to 0° C. 5-Chloro-2-methylnicotinoyl chloride (step 1) (2.20 g, 11.05 mmol) was slowly added portionwise and the reaction mixture was stirred at RT for 2 hours. The reaction mixture was partitioned between EtOAc and 1M HCl. The organic phase was washed with water and brine, dried (MgSO₄) filtered and the solvent was removed in vacuo to afford the title product which was used in the next step without further purification. ¹H NMR (400 MHz, DMSO-d₆) δ 8.53 (1H, d), 7.42 (1H, 5), 7.80 (1H, d), 3.70 (1H, m), 3.60 (3H, s), 2.49 (3H, s), 2.29 (1H, m), 1.95 (4H, m), 1.42 (2H, m), 1.29 (2H, m); [M+H]+311.26.

### Step 3: Trans-5-Chloro-N-(4-hydroxymethyl-cyclohexyl)-2-methyl-nicotinamide

Trans-4-[(5-Chloro-2-methyl-pyridine-3-carbonyl)-amino]-cyclohexanecarboxylic acid methyl ester (step 2) (2.20 g, 7.08 mmol) was placed in a flask with dry THF (100 ml). This was cooled to 0° C. and lithium aluminum hydride (0.537 g, 14.16 mmol) was added. The reaction mixture was stirred at RT for 2 hours and then quenched with water (0.5 ml), 2M NaOH (0.5 ml) and then water again (1.5 ml). The solids were filtered off through Celite® (filter material) and the filtrate was partitioned between EtOAc and water. The organic phase was washed with water and brine, dried over MgSO₄, filtered and the solvent was removed in vacuo to afford the title product which was used in the next step without further purification. ¹H NMR (400 MHz, DMSO-d₆) δ 8.53 (1H, d), 8.38 *1H, d), 7.79 (1H, d), 4.40 (1H, t), 3.66 (1H, m), 3.21 (2H, t), 2.47 (3H, s), 1.92 (2H, m), 1.78 (2H, m), 1.31 (1H, m), 1.22 (2H, m), 0.98 (2H, m). [M+H]+ 283.30.

### Step 4: Trans-toluene-4-sulfonic acid 4-[(5-chloro-2-methyl-pyridine-3-carbonyl)-amino]-cyclohexylmethyl ester

To a stirring solution of trans-5-chloro-N-(4-hydroxymethyl-cyclohexyl)-2-methyl-nicotinamide (step 3) (250 mg, 0.884 mmol) in DCM (4 ml) was added pyridine (1 ml) followed by tosyl chloride (253 mg, 1.326 mmol). The reaction mixture was left to stir at RT overnight and then diluted with DCM. The mixture was washed with 1M HCl, water, brine, dried (MgSO₄) and concentrated in vacuo to afford a pale yellow solid. The solid was sonicated in 1:5 EtOAc:iso-hexane and more EtOAc was added until all solid went into solution. Iso-hexane was carefully added to give a cloudy suspension which was collected by filtration to give the title compound.

MS m/z 437.2 [M+H]+; Method 2minLC_{—}30_v002.

¹H NMR (400 MHz, CDCl3) δ 8.50 (1H, d), 7.80 (2H, d), 7.63 (1H, d), 7.39 (2H, d), 5.70 (1H, d), 3.89 (1H, m), 3.84 (2H, d), 2.63 (3H, s), 2.48 (3H, s), 2.13 (2H, m), 1.85 (2H, m), 1.71 (1H, m), 1.23 (2H, m), 1.11 (2H, m).

### Intermediate B

### Trans-toluene-4-sulfonic acid 4-(2-chloro-5-trifluoromethyl-benzoylamino)-cyclohexylmethyl ester

### Step 1: Trans-4-(2-Chloro-5-trifluoromethyl-benzoylamino)-cyclohexane carboxylic acid methyl ester

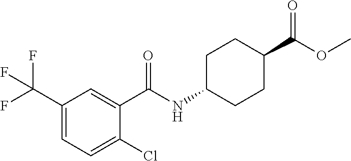

To a stirred suspension of trans-4-amino-cyclohexylcarboxylic acid methyl ester hydrochloride (6.7 g, 34.7 mmol) in dry THF (90 ml) under nitrogen atmosphere was added triethylamine (12 ml, 86.8 mmol). The suspension was cooled to 0° C. and 2-chloro-5-(trifluoromethyl)benzoyl chloride (8.85 g, 36.4 mmol) in dry THF (40 ml) was added dropwise over 20 minutes. The resulting thick, colourless slurry was stirred at 0-5° C. for 30 minutes and then allowed to warm to room temp and stirred at RT for 1 hour. The reaction was quenched by the dropwise addition of water (5 ml) in THF (45 ml) to give a clear solution. This was diluted with water (100 ml) and ethyl acetate (300 ml). The biphasic mixture was stirred for 5 minutes then the organic phase was separated and washed successively with water (100 ml), saturated sodium bicarbonate (100 ml) and saturated brine (100 ml), dried (MgSO₄), filtered and evaporated to give a colourless solid.; [M+H]+ 364.

### Step 2: Trans-2-Chloro-N-(4-hydroxymethyl-cyclohexyl)-5-trifluoromethyl-benzamide

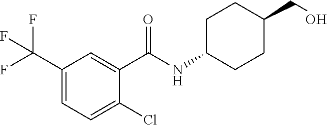

To a solution of trans-4-(2-chloro-5-trifluoromethyl-benzoylamino)-cyclohexane carboxylic acid methyl ester (step 1) (95.2 g, 0.26 mol) in dry THF (1 litre) under nitrogen at 0° C. was added lithium aluminium hydride pellets (20 g, 0.53 mol) portion wise over 3 hours. The reaction mixture was stirred at 0° C. for a further 2 hours and then carefully quenched at 0° C. by the addition of water (40 ml) in THF (60 ml) followed by further THF (500 ml) to maintain a mobile suspension. Finally, 1M sodium hydroxide solution (80 ml) was added at 0° C. resulting in a yellow solution containing a colourless suspension. The reaction was filtered through a Celite® pad (filter material) to remove inorganic salts. The Celite® pad/salts were washed with EtOAc (500 ml) then with EtOAc:THF (1:1; 300 ml). The organics were combined and diluted with further EtOAc (600 ml) and then washed with saturated brine (600 ml). The organic layer was dried (Na₂SO₄), filtered and concentrated under reduced pressure until a slurry was obtained. Et₂O was added to the slurry, which was then stirred for 5 minutes before being filtered to recover a colourless solid. The solid was washed with iso-hexane and then dried at 35° C. under vacuum to give the required product.

### Step 3: Trans-toluene-4-sulfonic acid 4-(2-chloro-5-trifluoromethyl-benzoylamino)-cyclohexylmethyl ester

Trans-2-Chloro-N-(4-hydroxymethyl-cyclohexyl)-5-trifluoromethyl-benzamide (step 2) (1 g, 2.98 mmol) was added to a mixture of DCM (12 ml) and pyridine (3.00 ml). Tosyl chloride (0.852 g, 4.47 mmol) was added and the mixture was stirred at RT. After diluting with DCM, the mixture was washed with 1M HCl, water, brine, dried (MgSO₄) and concentrated in vacuo to afford a pale yellow solid. The solid was triturated with EtOAc:iso-hexane to afford the titled product; LC-MS Rt 1.33 mins; MS m/z 490.1 [M+H]+; Method 2minLC_{—}30. ¹H NMR (400 MHz, DMSO-d6) δ 8.49 (1H, d), 7.80 (3H, m), 7.74 (2H, m), 7.50 (2H, m), 3.85 (2H, d), 3.62 (1H, m), 2.43 (3H, s), 1.89 (2H, m), 1.66 (2H, m), 1.58 (1H, m), 1.21 (2H, m), 1.02 (2H, m).

### Intermediate C

### Trans-methanesulfonic acid 4-(2-chloro-5-trifluoromethyl-benzoylamino)-cyclohexyl methyl ester

Trans-2-chloro-N-(4-hydroxymethyl-cyclohexyl)-5-trifluoromethyl-benzamide (Int. B, step 2) (1 g, 2.98 mmol) was suspended in DCM (25 ml). THF (6 ml) was added to solubilise the alcohol. The mixture was cooled to 0° C. and treated with triethylamine (0.623 ml, 4.47 mmol) followed by dropwise addition of methanesulfonyl chloride (0.255 ml, 3.28 mmol). The reaction mixture was allowed to warm to RT overnight. After diluting with DCM, the mixture was washed with 1M HCl, water, brine, dried (MgSO₄) and concentrated in vacuo to afford the title compound as a white solid; ¹H NMR (400 MHz, CDCl3) δ 7.82 (1H, s), 7.53 (1H, d), 7.45 (1H, d), 5.91 (1H, d), 4.00 (2H, d), 3.90 (1H, m), 2.94 (3H, s), 2.14 (2H, m), 1.86 (2H, m), 1.71 (1H, m), 1.19 (4H, m).

### Intermediate D

### Trans-Methanesulfonic acid 4-[(5-chloro-2-methyl-pyridine-3-carbonyl)-amino]-cyclohexylmethyl ester

A solution of trans-5-chloro-N-(4-hydroxymethyl-cyclohexyl)-2-methyl-nicotinamide (Int. A step 3) (100 mg, 0.354 mmol) and pyridine (3.6 ml) in dry DCM (3.5 ml) under nitrogen was cooled to approx. 0° C. using an ice-water bath. Methanesulfonyl chloride (0.030 ml, 0.389 mmol) was added dropwise. The reaction mixture was allowed to warm to RT and stirred for 4 hours. The reaction was quenched by the addition of sat. NH₄Cl at RT and then extracted with diethyl ether (3×20 ml). The Et₂O extracts were combined, washed with sat. brine (20 ml), dried (MgSO₄), filtered and evaporated to give the title compound as a colourless solid. LC-MS m/z 361.2/363.2 [M+H]+. ¹H NMR (400 MHz, CDCl₃) δ 8.52 (1H, d), 7.65 (1H, d), 5.68 (1H, br d), 4.09 (2H, d), 3.96 (1H, m), 3.04 (3H, s), 2.65 (3H, s), 2.21 (2H, m), 1.96 (2H, m), 1.79 (1H, m), 1.27 (4H, m).

### Intermediate E

### Trans-trifluoro-methanesulfonic acid 4-(2-chloro-5-trifluoromethyl-benzoylamino)-cyclohexylmethylester

Trans-2-Chloro-N-(4-hydroxymethyl-cyclohexyl)-5-trifluoromethyl-benzamide (Int. B, step 2) (2.00 g, 5.96 mmol) was placed in a flask with DCM (50 ml) and pyridine (0.56 g, 7.15 mol). The reaction mixture was cooled to 0° C. and then triflic anhydride (1.85 g, 6.55 mmol) was added dropwise. The mixture was stirred at 0° C. for 1 hour and partitioned between DCM and 1M HCl. The organic phase was dried (MgSO₄), filtered and the solvent was removed in vacuo on a ice-cold water bath to give a beige solid. The crude product was triturated in iso-hexane:Et₂O—2:1 to afford the title product. ¹H NMR (d6-DMSO, 400 MHz) δ 8.52 (1H, d), 7.81 (1H, m), 7.74 (2H, m), 4.11 (2H, d), 3.70 (1H, m), 1.95 (2H, m), 1.79 (2H, m), 1.67 (1H, m), 1.28 (2H, m), 1.11 (2H, m).

### Intermediate F

### 6-Chloro-3,3-difluoroindolin-2-one

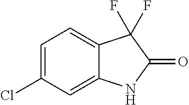

A suspension of 6-chloroindoline-2,3-dione (250 mg, 1.377 mmol) in DCM (14 ml) was treated with Deoxo-Fluoro(R) (50% in toluene, 1.099 ml, 3.44 mmol) over 10 minutes. After addition the suspension dissolved and the mixture was left at RT overnight. The reaction was quenched by addition of sat. aq. NaHCO₃ (6 ml) and the organic portion was separated and concentrated in vacuo. Purification by chromatography on silica eluting with DCM (100%) afforded the title product as an off white solid; ¹H NMR (400 MHz, d6-DMSO) δ 11.36 (1H, s), 7.70 (1H, dt), 7.22 (1H, dd), 7.03 (1H, m).

5-Chloro-3,3-difluoroindolin-2-one was made in an analogous way. ¹H NMR (400 MHz, d6-DMSO) δ 11.32 (1H, s), 7.84 (1H, m), 7.58 (1H, m), 7.01 (1H, m)]

### Intermediate G

### 3,3-Dimethyl-1,3-dihydro-pyrrolo[2,3-b]pyridin-2-one

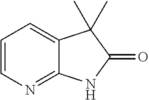

### Step 1: 3,3-Dibromo-1,3-dihydro-pyrrolo[2,3-b]pyridin-2-one

To a solution of 7-azaindole (2.51 g, 21.25 mmol) in tert-BuOH (150 ml) at 25° C. was added pyridine hydrobromide perbromide (23.03 g, 64.8 mmol) in portions over 30 minutes and stirred for 2.5 hours. The solvent was removed in vacuo diluted with EtOAc (400 ml) and washed with water (400 ml). The aqueous phase was back extracted with EtOAc (200 ml) and the combined organics were washed with brine (50 ml), dried (Na₂SO₄), filtered and concentrated in vacuo. The resulting solid was triturated with DCM (˜30 ml) and filtered to afford the title compound; LC-MS MS m/z 293.1 [M+H]+.

### Step 2: 1,3-Dihydro-pyrrolo[2,3-b]pyridin-2-one

To a solution of 3,3-dibromo-1,3-dihydro-pyrrolo[2,3-b]pyridin-2-one (4.6 g, 15.76 mmol) in AcOH (80 ml) under N₂ was added zinc powder (10.30 g, 10 eq). The mixture was stirred at RT for 30 min under N₂ and then filtered through Celite® (filter material) to remove the Zn. AcOH was removed in vacuo and the mixture was diluted with EtOAc and washed with NaHCO₃. The organic phase was separated dried (MgSO₄) and concentrated in vacuo to afford the title product; ¹H NMR (400 MHz, DMSO-d6) δ 10.95 (1H, s), 8.05 (1H, d), 7.55 (1H, d), 6.95 (1H, t), 3.55 (2H, s).

### Step 3: 3,3-Dimethyl-1,3-dihydro-pyrrolo[2,3-b]pyridin-2-one

1,3-dihydro-pyrrolo[2,3-b]pyridin-2-one (500 mg, 3.73 mmol) in dry THF (40 ml) under at atmosphere of nitrogen was treated with N1,N1,N2,N2-tetramethylethane-1,2-diamine (1.956 ml, 13.05 mmol). The mixture was cooled to −78° C. in an acetone/dry ice bath. n-BuLi (1.6M in Hexanes) (8.15 ml, 13.05 mmol) was added dropwise over 30 mins. After addition, the mixture was stirred for a further 30 mins and then treated dropwise with methyl iodide (0.816 ml, 13.05 mmol) and stirred at RT overnight. The reaction was quenched by careful addition of NH₄Cl (20 ml) and the mixture was extracted with EtOAc (2×75 ml). The organic portion was separated and washed with sat NaHCO₃, brine, dried (MgSO₄) and concentrated in vacuo to give a pale yellow powder. The methylation process was repeated twice to obtain the dimethylated product. Purification of the resulting solid by chromatography on silica eluting with 0% to 50% EtOAc in iso-hexane afforded the title product; ¹H NMR (400 MHz, DMSO-d6) δ 10.95 (1H, s), 8.05 (1H, dd), 7.65 (1H, dd), 6.95 (1H, dd), 1.25 (6H, s).

### Intermediate GB

### 6-chloro-3,3-dimethylindolin-2-one

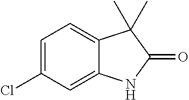

The title compound is prepared from commercially available 6-chloro-1,3-dihydro-indol-2-one analogously to 3,3-Dimethyl-1,3-dihydro-pyrrolo[2,3-b]pyridin-2-one (Intermediate G step 3); ¹H NMR (400 MHz, CDCl3) δ 7.50 (1H, br), 7.10 (1H, d), 7.05 (1H, d), 6.90 (1H, s), 1.40 (6H, s).

### Intermediate H

### 5′-Fluorospiro[cyclopropane-1,3′-indolin]-2′-one

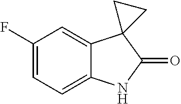

5-Fluoroindolin-2-one (500 mg, 3.31 mmol) was dissolved in dry THF (30 ml). To this was added N1,N1,N2,N2-tetramethylethane-1,2-diamine (1.091 ml, 7.28 mmol) and the mixture was cooled to −78° C. BuLi (1.6M in Hexanes) (4.14 ml, 6.62 mmol) was added dropwise and the contents left stirring for 20 mins. 1,2-Dibromoethane (0.342 ml, 3.97 mmol) was added dropwise and the mixture was stirred at −78° C. for 30 mins and allowed to warm to RT overnight. The reaction was quenched by addition of NH₄Cl (20 ml) and extracted into EtOAc. The solvent was removed in vacuo and purification by chromatography on silica eluting with acetone/iso-hexane afforded the title compound as a white solid; ¹H NMR (400 MHz, CDCl₃) δ 8.40 (1H, s), 6.90 (2H, m), 6.60 (1H, d), 1.80 (2H, m), 1.55 (2H, m).

### Intermediate I

### 5-Methoxy-3,3-dimethyl-1,3-dihydro-indol-2-one

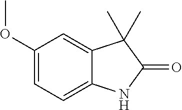

### Step 1: 5-Methoxy-indole-1-carboxylic acid tert-butyl ester

To a solution of 5-methoxyindole (4.11 g, 27.9 mmol) in MeCN was added di-tert-butyl dicarbonate (7.13 ml, 30.7 mmol) followed by DMAP (0.102 g, 0.838 mmol): The reaction mixture was stirred at RT for 64 hours. The reaction mixture was partitioned between EtOAc (75 ml) and cold 1M HCl (50 ml), extracted with EtOAc (2×50 ml) and the combined organics washed with brine (2×50 ml). The organic portion was dried (Na₂CO₃), filtered and concentrated in vacuo to yield a white solid. The solid was triturated with iso-hexane (15 ml) to afford the title compound as a white solid; ¹H NMR (400 MHz, CDCl3) δ 8.04 (1H, br d), 7.59 (1H, d), 7.05 (1H, d), 6.95 (1H, dd), 6.52 (1H, d), 3.88 (3H, s), 1.69 (9H, s).

### Step 2: 1-(tert-Butoxycarbonyl)-5-methoxy-1H-indol-2-ylboronic acid

LDA Solution: To a solution of diisopropylamine (3.82 ml, 26.8 mmol) at −78° C. in THF (3 ml) was added butyllithium (2.5M in hexane) (10.73 ml, 26.8 mmol). After 10 mins the solution was warmed to 0° C. and stirred at this temperature for 10 mins.

To a solution of 5-methoxy-indole-1-carboxylic acid tert-butyl ester (5.53 g, 22.36 mmol) and triisopropyl borate (7.79 ml, 33.5 mmol) in THF (17 ml) at 0° C. was added dropwise the LDA solution prepared above (added over 10 minutes). The reaction mixture was stirred at 0° C. for 1 hour and quenched by the addition of 2N HCl (35 ml). The reaction was extracted with dichloromethane (3×50 ml), washed brine (50 ml), dried (MgSO₄) and concentrated in vacuo. The resulting oil was dissolved in Et₂O: iso-hexane 1:1 and scratched to induce crystallization. The resulting solid was filtered off to yield the title product as a white solid: ¹H NMR (400 MHz, DMSO-d6) δ 8.16 (2H, s), 7.95 (1H, d), 7.08 (1H, d), 6.87 (1H, dd), 6.55 (1H, s), 3.77 (3H, s), 1.59 (9H, s).

### Step 3: 5-Methoxy-2-oxo-2,3-dihydro-indole-1-carboxylic acid tert-butyl ester

To a suspension of 1-(tert-butoxycarbonyl)-5-methoxy-1H-indol-2-ylboronic acid (2.10 g, 7.21 mmol) in acetone (16.00 ml), water (16 ml) and THF (8 ml) was added followed by sodium hydroxide (0.433 g, 10.82 mmol) and sodium bicarbonate (4.85 g, 57.7 mmol). The reaction mixture was cooled to 0° C., then oxone (4.43 g, 7.21 mmol) was added and the reaction stirred at 0° C. for 30 minutes. The reaction was quenched by the addition of 1M Na₂S₂O₅ (100 ml) and then the mixture was partitioned between EtOAc (100 ml), extracted with EtOAc (3×20 ml) and the combined organics washed with brine (1×75 ml). The organic portion was dried (MgSO₄), filtered and concentrated in vacuo to yield a yellow solid.

The residue was crystallized from hot EtOAc. On cooling, iso-hexane was added to yield the title compound as a cream solid; ¹H NMR (400 MHz, DMSO-d6) δ 7.60 (1H, d), 6.93 (1H, d), 6.86 (1H, dd), 3.74 (3H, s), 3.71 (2H, s), 1.55 (9H, s).

### Step 4: 5-Methoxy-1,3-dihydro-indol-2-one ester

To a solution of 5-methoxy-2-oxo-2,3-dihydro-indole-1-carboxylic acid tert-butyl ester (0.800 g, 3.04 mmol) in dichloromethane (6 ml) at 0° C. was added trifluoroacetic acid (0.234 ml, 3.04 mmol). The reaction mixture was stirred at 0° C. for 1 hour and quenched by pouring into sat NaHCO₃ (50 ml). The aqueous layer was back-extracted with EtOAc (3×35 ml). The combined organic extracts were washed with brine (1×50 ml) dried (MgSO₄) and concentrated in vacuo to yield a solid residue. The residue was crystallized from hot EtOAc (4 ml) and iso-hexane (2 ml) was added on cooling to yield the title compound as a pink solid: LC-MS Rt=0.54 mins; MS m/z 164.0 [M+H]+; Method 2minLC_{—}30_v002; ¹H NMR (400 MHz, DMSO-d6) δ 10.20 (1H, br s), 6.86 (1H, s), 6.72 (2H, m), 3.69 (3H, s), 3.43 (2H, s).

### Step 5: 5-Methoxy-3,3-dimethyl-1,3-dihydro-indol-2-one

The title compound was prepared from 5-methoxy-1,3-dihydro-indol-2-one ester analogously to 3,3-dimethyl-1,3-dihydro-pyrrolo[2,3-b]pyridin-2-one (Intermediate G step 3);). This step was repeated twice to obtain the dimethylated product. ¹H NMR (400 MHz, CDCl3) δ 7.80 (1H, br), 6.85 (2H, m), 6.70 (1H, d), 3.80 (3H, s), 1.40 (6H, s).

### Intermediate IB

### 3,3,7-Trimethylindolin-2-one

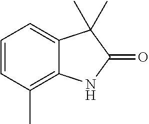

This compound was prepared analogously to Intermediate I by replacing 5-methoxyindole (step 1) with 7-methyl-1H-indole; LC-MS Rt=2.32 mins; MS m/z 176.16[M+H]+; Method LowpH_v002

### Intermediate IC

### 3,3,4-trimethylindolin-2-one

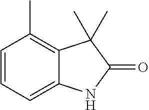

This compound was prepared analogously to Intermediate I by replacing 5-methoxyindole (step 1) with 4-methyl-1H-indole; 1HNMR (400 MHz, DMSO-d6) δ 10.3 (1H, s), 7.05 (1H, t), 6.7 (1H, d), 6.65 (1H, d), 2.3 (3H, s), 1.3 (6H, s).

### Intermediate ID

### 7-Chloro-3,3-dimethyl-1,3-dihydro-indol-2-one

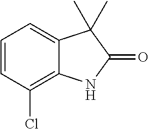

This compound was prepared analogously to Intermediate I by replacing 5-methoxyindole (step 1) with 7-chloroindole; 1H NMR (400 MHz, DMSO-d6) δ 10.75 (1H, s), 7.25 (2H, dd), 6.95 (1H, t), 1.25 (6H, s).

### Intermediate J

### 6-Methoxy-3,3-dimethyl-1,3-dihydro-pyrrolo[3,2-c]pyridin-2-one

The title compound was prepared from 6-methoxy-1H-pyrrolo[3,2-c]pyridin-2(3H)-one (comm. avail.) analogously to 3,3-dimethyl-1,3-dihydro-pyrrolo[2,3-b]pyridin-2-one (Intermediate G step 3). This step was repeated twice to obtain the dimethylated product. ¹H NMR (400 MHz, CDCl3). δ 7.90 (1H, s), 6.30 (1H, s), 3.95 (3H, s), 1.45 (6H, s).

### Intermediate KA and KB

### (R)-3-Fluoro-3-methyl-1,3-dihydro-indol-2-one and (S)-3-Fluoro-3-methyl-1,3-dihydro-indol-2-one

### Step 1: (R/S)-3-Hydroxy-3-methyl-1,3-dihydro-indol-2-one

A solution of isatin (3 g, 20.39 mmol) in THF (90 ml) was cooled in an acetone/dry ice bath and MeMgBr (3M, 20.39 ml) was added slowly over 15 minutes. The reaction mixture was stirred vigorously for 1 h 45 min and then removed from the acetone/dry ice bath and sat. aq. NH₄Cl (5 ml) was added. The reaction mixture was stirred until all the gas had evolved and a further sat. aq. NH₄Cl (15 ml) was added. Water was added to dissolve the solids and the mixture was extracted with EtOAc (70 ml). The combined organic extracts were washed with brine (30 ml), dried (Na₂SO₄) and concentrated in vacuo. To the resulting yellow solid was added DCM (15 ml) and the solid filtered off and dried in a vacuum oven at 40° C. for 1.5 h to afford the title compounds.

### Step 2: (R)-3-Fluoro-3-methyl-1,3-dihydro-indol-2-one and (S)-3-Fluoro-3-methyl-1,3-dihydro-indol-2-one

To a suspension of (R/S)-3-hydroxy-3-methyl-1,3-dihydro-indol-2-one (1 g, 6.13 mmol) in DCM (65 ml) at −78° C. was added Deoxo-Fluoro(R) (50% in toluene, 2.445 ml, 7.66 mmol) over 10 minutes and the mixture warmed to RT overnight. The reaction was quenched by addition of sat. aq. NaHCO₃ (6 ml) and the organic portion was separated and concentrated in vacuo. Purification by chromatography on silica eluting with 0-40% EtOAc in iso-hexanes afforded the title products as a mixture. The mixture was separated by chiral SFC to give the following compounds:

(R)-3-Fluoro-3-methyl-1,3-dihydro-indol-2-one data: ¹H NMR (400 MHz, d6-DMSO) δ 10.65 (1H, s), 7.49, (1H, d), 7.34 (1H, t), 7.05 (1H, t), 6.89 (1H, d), 1.66 (3H, d)

(S)-3-Fluoro-3-methyl-1,3-dihydro-indol-2-one ¹H NMR (400 MHz, d6-DMSO) δ 10.64 (1H, s), 7.49, (1H, d), 7.34 (1H, t), 7.05 (1H, t), 6.89 (1H, d), 1.66 (1H, d)

### Intermediate KC and KD

### (R)-3-Fluoro-3,5,6-trimethyl-1,3-dihydro-indol-2-one and (S)-3-Fluoro-3,5,6-trimethyl-1,3-dihydro-indol-2-one

The title compounds were prepared analogously to Intermediates KA and KB by replacing isatin with 5,6-dimethyl-1H-indole-2,3-dione; Separation by chiral SFC afforded the following compounds:

Intermediate KC (Enantiomer 1):

¹H NMR (400 MHz, CDCl₃) δ 8.07 (1H, bs), 7.10 (1H, s), 6.64 (1H, s), 2.19 (3H, app-s), 2.17 (3H, s), 1.68 (3H, d).

Intermediate KD (Enantiomer 2):

¹H NMR (400 MHz, CDCl₃) δ 8.10 (1H, bs), 7.10 (1H, s), 6.65 (1H, s), 2.19 (3H, app-s), 2.17 (3H, s), 1.68 (3H, d).

### Intermediate KE and KF

### (R)-6-chloro-3-fluoro-3-methylindolin-2-one and (S)-6-chloro-3-fluoro-3-methylindolin-2-one

The title compounds were prepared analogously to Intermediates KA and KB by replacing isatin with 6-chloroisatin. Separation by chiral SFC afforded the following compounds:

Intermediate KE (Enantiomer 1):

[M−F]+ ion 180.1 Rt 2.25 min

Intermediate KF (Enantiomer 2):

[M−F]+ ion 180.1 m/z at 2.25 min

### Intermediate KG

### 3,5,6-Trifluoro-3-methylindolin-2-one

The title compound is prepared from commercially available 5,6-difluoro-1H-indole-2,3-dione analogously to Intermediate KA steps 1 and 2. No chiral SFC required.

### Intermediate L

### 3,3-Dimethyl-5-trifluoromethoxy-1,3-dihydro-indol-2-one

The title compound was prepared from 5-(trifluoromethoxy)indolin-2-one (comm. avail.) analogously to 3,3-dimethyl-1,3-dihydro-pyrrolo[2,3-b]pyridin-2-one (Intermediate G step 3); ¹H NMR (400 MHz, CDCl3) δ 0.8.3 (1H, br), 7.10 (2H, m), 6.90 (1H, d), 1.40 (6H, s).

### Intermediate M

### 5-Fluoro-3,3-dimethyl-1,3-dihydro-indol-2-one

The title compound was prepared from 5-fluoroindolin-2-one (comm. avail.) analogously to 3,3-dimethyl-1,3-dihydro-pyrrolo[2,3-b]pyridin-2-one (Intermediate G step 3); ¹H NMR (400 MHz, CDCl3) δ 0.7.9 (1H, br), 6.9 (2H, m), 6.8 (1H, m), 1.4 (6H, s).

### Intermediate N

### 4-Methoxy-3,3-dimethyl-1,3-dihydro-indol-2-one

### Step 1: 4-Methoxy-1,3-dihydro-indol-2-one

This compound was prepared analogously to 5-methoxy-1,3-dihydro-indol-2-one ester (Intermediate I step 4) by replacing of 5-methoxyindole (step 1) with 4-methoxyindole; LC-MS Rt=1.85 mins; MS m/z 164.16 [M+H]+; Method LowpH_v002

### Step 2: 4-Methoxy-3,3-dimethyl-1,3-dihydro-indol-2-one

To a solution of 4-methoxy-1,3-dihydro-indol-2-one (1.556 g, 9.54 mmol) in THF (10 ml) at 0° C. was added methyl iodide (1.8 ml, 3 eq) followed by NaH (839 mg, 2.2 eq). The reaction mixture was stirred at 0° C. for 1 h. The reaction was quenched by the addition of sat NH₄Cl and partitioned between EtOAc (1 ml) and H₂O (5 ml). The mixture was extracted with EtOAc (2×10 ml) and the combined organic extracts were washed with brine (1×20 ml), dried (MgSO₄) and concentrated in vacuo. Purification of the crude residue by chromatography on silica eluting with

0% to 30% iso-hexane: EtOAc afforded the title product; ¹H NMR ((400 MHz, DMSO-d6) δ 10.25 (1H, s), 7.15 (1H, t), 6.65 (1H, d), 6.45 (1H, d), 3.8 (3H, s), 1.3 (6H, s).

### Intermediate O

### 7-Chloro-3,3-dimethyl-1,3-dihydro-indol-2-one

This compound was prepared analogously 4-methoxy-3,3-dimethyl-1,3-dihydro-indol-2-one (Intermediate N) by replacing of 4-methoxyindole (step 1) with 7-chloroindole; ¹H NMR ((400 MHz, DMSO-d6) δ 10.75 (1H, s), 7.25 (2H, dd), 6.95 (1H, t), 1.25 (6H, s).

### Intermediate P

### 3,3-Difluoro-1,3-dihydro-indol-2-one

This compound was prepared from 1H-Indole-2,3-dione analogously to 6-chloro-3,3-difluoroindolin-2-one (Intermediate F); ¹H NMR (400 MHz, d6-DMSO) δ 11.17 (1H, s), 7.64 (1H, dd), 7.52 (1H, t), 7.16 (1H, t), 6.99 (1H, m)

Further indol-2-ones used in the preparation of Examples were synthesised from commercially available starting compounds analogously to Intermediates F, G, H, I, J, K or N.

### Intermediate Q

### 5-Methoxy-3,4-dihydro-2H-isoquinolin-1-one

To a stirring solution of 5-hydroxy-3,4-dihydroisoquinolin-1(2H)-one (200 mg, 1.226 mmol) in DMF (8 ml) was added Cs₂CO₃ (599 mg, 1.839 mmol). The reaction mixture was left to stir for 20 minutes at 50° C. and then treated with methyl iodide (0.115 ml, 1.839 mmol). After stirring at 50° C. for 30 min, the mixture was diluted with EtOAc/water. The organic portion was separated and washed with water, brine, dried (MgSO₄) and concentrated in vacuo to afford the title compound as a pale yellow solid; LC-MS Rt=1.08 mins; MS m/z 178.1 [M+H]+; Method 2minLC_v002.

### Intermediate RA

### 1-Ethyl-1,3-dihydro-benzoimidazol-2-one

### Step 1: N-Ethyl-benzene-1,2-diamine

N-Ethyl-2-nitroaniline (1 g, 6.02 mmol) and ammonium formate (1.897 g, 30.1 mmol) were dissolved in ethanol (20 ml). Pd/C (10% Carbon on Pd, 100 mg, 0.094 mmol) was added and the reaction was heated at reflux for 1 hour. The mixture was filtered and washed through with MeOH. The filtrate was concentrated in vacuo to afford the title compound as an oil; ¹H NMR (400 MHz, CDCl3) δ 7.29 (1H, s), 6.85 (1H, m), 6.75 (1H, m), 6.70 (2H, m), 3.45 (2H, s, broad), 3.18 (2H, q), 1.33 (3H, t).

### Step 2: 1-Ethyl-1,3-dihydro-benzoimidazol-2-one

N-Ethyl-benzene-1,2-diamine (0.795 g, 5.84 mmol) was dissolved in THF (25 ml) and to this solution, CDI (0.947 g, 5.84 mmol) was added. The resulting solution was stirred at RT under an atmosphere of N₂ overnight and then at 50° C. for 3 hours. The solvent was removed in vacuo and the resulting crude was purified by chromatography on silica eluting with iso-hexane/EtOAc to afford the title product; ¹H NMR (400 MHz, DMSO) δ 10.89 (1H, s), 7.14 (1H, m), 6.97 (3H, m), 3.80 (2H, q), 1.18 (3H, t).

### Intermediates RB-RF

These intermediates namely,

4-Methyl-1,3-dihydro-imidazo[4,5-b]pyridin-2-one (Intermediate RB),

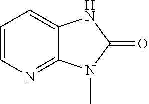

1,3-Dihydro-benzoimidazol-2-one (Intermediate RC),

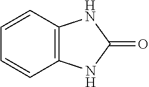

1-Methyl-1,3-dihydro-benzoimidazol-2-one (Intermediate RD),

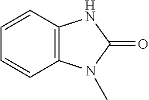

and 1-Ethyl-1,3-dihydro-benzoimidazol-2-one (Intermediate RE),

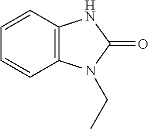

were prepared analogously to Intermediate RA by replacing N-ethyl-2-nitroaniline with the appropriate starting compound.

### Intermediate RF

### 2-Oxo-2,3-dihydro-benzoimidazole-1-carboxylic acid tert-butyl ester

1,3-Dihydro-benzoimidazol-2-one (Intermediate RD) (1 g, 7.46 mmol) was dissolved in dry DMF (20 ml) under nitrogen and treated with NaH (0.328 g, 8.20 mmol) portionwise. After 1.5 h, a solution of di-tert-butyl dicarbonate (1.627 g, 7.46 mmol) in DMF (10 ml) was added dropwise and the mixture stirred at RT for 4 h. The solvent was removed in vacuo and the mixture was partitioned between sat. NH₄Cl (˜50 ml) and EtOAc (˜100 ml). The aqueous portion was extracted with EtOAc (˜100 ml) and the combined organic extracts were dried (MgSO₄) and concentrated in vacuo. Trituration with ethyl acetate/iso-hexane afforded the title compound; ¹H NMR (400 MHz, DMSO) δ 11.21 (1H, s), 7.65 (1H, m), 7.14 (1H, m), 7.06 (1H, m), 6.99 (1H, m), 1.59 (9H, s).

### Intermediate RG

### 1-Methyl-1,3-dihydro-imidazo[4,5-b]pyridin-2-one

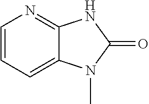

### Step 1: N-methyl-2-nitropyridin-3-amine

3-Methoxy-2-nitropyridine (2 g, 12.98 mmol) was dissolved in 2M methylamine in MeOH (30 ml, 60.0 mmol) and heated using microwave radiation at 120° C. for 2 hours. The solvent was removed in vacuo and the residue was partitioned between DCM and water. The organic portion was separated and the aqueous was extracted with DCM. The combined organic extracts were washed with saturated brine solution, dried (MgSO₄) and concentrated in vacuo. Purification of the crude residue by chromatography on silica eluting with DCM:MeOH followed by recrystallisation of the product from iso-hexane:EtOAc yielded the title compound as a solid; ¹H NMR (400 MHz, DMSO) δ 7.91 (1H, s), 7.82 (1H, d), 7.64 (1H, m), 7.56 (1H d), 2.94 (3H, d).

### Step 2: N3-methylpyridine-2,3-diamine

N-methyl-2-nitropyridin-3-amine (535 mg, 3.49 mmol) was dissolved in MeOH (50 ml) and treated with Pd—C (100 mg, 0.094 mmol). The solution was placed under a positive pressure of H₂ for 4 hours and then filtered. The filtrate was concentrated in vacuo to afford the title compound which was used in the next step without further purification; ¹H NMR (400 MHz, DMSO) δ 7.28 (1H, m), 6.50 (2H, m), 5.36 (2H, s), 4.84 (1H, m), 2.69 (3H, d).

### Step 3: 1-Methyl-1,3-dihydro-imidazo[4,5-b]pyridin-2-one

The title compound was prepared from N3-methylpyridine-2,3-diamine analogously to 1-ethyl-1,3-dihydro-benzoimidazol-2-one (Intermediate RA); ¹H NMR (400 MHz, DMSO) δ 11.49 (1H, s), 7.91 (1H, m), 7.41 (1H, m), 7.02 (1H, m), 3.30 (3H, s).

### Intermediate RH

### 3,6-Dimethyl-1,3-dihydro-imidazo[4,5-b]pyridin-2-one

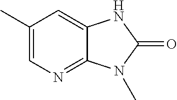

### Step 1: Methyl-(5-methyl-3-nitro-pyridin-2-yl)-amine

2-Chloro-5-methyl-3-nitropyridine (1.00 g, 5.79 mmol) was placed in a microwave vial with methylamine (2M in THF) (11.59 ml, 23.2 mmol) and the reaction mixture was heated using microwave radiation at 100° C. for 30 minutes. The solvent was removed in vacuo and the resulting residue was taken up in EtOAc. Any undissolved solid was filtered off and the solvent was removed in vacuo to afford the title compound which was used in the next step without further purification.

¹H NMR (400 MHz, DMSO) δ 8.40 (1H, s), 8.33 (1H, br), 8.25 (1H, s), 3.02 (3H, d), 2.21 (3H, s).

### Step 2: N2,5-dimethylpyridine-2,3-diamine

This compound was prepared from methyl-(5-methyl-3-nitro-pyridin-2-yl)-amine (Step 1) analogously to N-ethyl-benzene-1,2-diamine (Intermediate RA step 1).

¹H NMR (400 MHz, DMSO) δ 8.40 (1H, s), 8.33 (1H, br), 8.25 (1H, s), 3.02 (3H, d), 2.21 (3H, s).

### Step 3: 3,6-Dimethyl-1,3-dihydro-imidazo[4,5-b]pyridin-2-one

This compound was prepared from N2,5-dimethylpyridine-2,3-diamine (step 2) analogously to 1-ethyl-1,3-dihydro-benzoimidazol-2-one (Intermediate RA step 2).

¹H NMR (400 MHz, DMSO) δ 11.00 (1H, broad), 7.78 (1H, d), 7.12 (1H, d), 3.28 (3H, s), 2.29 (3H, s)

### Intermediate RI

### 3-Ethyl-1,3-dihydro-imidazo[4,5-c]pyridin-2-one

This compound was prepared from N3-ethylpyridine-3,4-diamine (commercially available) analogously to 1-ethyl-1,3-dihydro-benzoimidazol-2-one (Intermediate RA step 2). LC-MS Rt=1.74 mins; MS m/z 164.1 [M+H]+; Method LowpH_v002.

### Intermediate RJ

### 3,7-Dimethyl-1,3-dihydro-imidazo[4,5-b]pyridin-2-one

This compound was prepared from N2,4-dimethylpyridine-2,3-diamine (commercially available) analogously to 1-ethyl-1,3-dihydro-benzoimidazol-2-one (Intermediate RA step 2). LC-MS Rt=1.59 mins; MS m/z 164.06 [M+H]+; Method LowpH_v002.

### Intermediate RK-RN

These intermediates namely,

3,5-Dimethyl-1,3-dihydro-imidazo[4,5-b]pyridin-2-one (Intermediate RK),

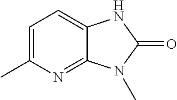

1,5-Dimethyl-1,3-dihydro-imidazo[4,5-b]pyridin-2-one (Intermediate RL),

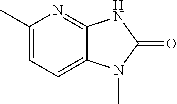

1-Ethyl-1,3-dihydro-benzoimidazol-2-one (Intermediate RM),

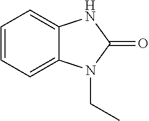

5-Methoxy-3-methyl-1,3-dihydro-imidazo[4,5-b]pyridin-2-one (Intermediate RN),

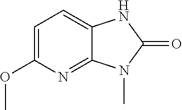

are prepared from the appropriate commercially available starting compound analogously to 1-ethyl-1,3-dihydro-benzoimidazol-2-one (Intermediate RA step 2).

### Intermediate RO

### 1-Isobutyl-1,3-dihydro-benzoimidazol-2-one

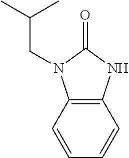

### Step 1: tert-Butyl 3-isobutyl-2-oxo-2,3-dihydro-1H-benzo[d]imidazole-1-carboxylate

2-Oxo-2,3-dihydro-benzoimidazole-1-carboxylic acid tert-butyl ester (Intermediate RA) (200 mg, 0.854 mmol) was dissolved in DMF (3 ml) and stirred under N₂ at RT. Sodium Hydride (60% in mineral oil) (41.0 mg, 1.025 mmol) was added and mixture was stirred for 20 mins. 1-Iodo-2-methylpropane (0.147 ml, 1.281 mmol) was added and the mixture was stirred at RT for 2 days. The mixture was heated to 50° C. After 2 hrs 1 equivalence of NaH was added followed by 0.5 equivalence of 1-iodo-2-methylpropane and stirring continued for a further 1 hr 30 mins. The solvent was removed in vacuo and the residue was partitioned between DCM (˜40 ml) and water (˜5 ml). The organic portion was passed through a phase separator and the solvent was removed in vacuo. Purification by chromatography on silica eluting with iso-hexane/EtOAc afforded the title compound.

¹H NMR (400 MHz, d6-DMSO) δ 7.72 (1H, d), 7.23 (2H, m), 7.12 (1H, t), 3.62 (2H, d), 2.11 (1H, m), 1.60 (9H, s), 0.90 (6H, d).

### Step 2: 1-Isobutyl-1,3-dihydro-benzoimidazol-2-one

### tert-Butyl 3-isobutyl-2-oxo-2,3-dihydro-1H-benzo[d]imidazole-1-carboxylate (151 mg, 0.520 mmol) in MeOH (4 ml) was treated with 4M HCl/dioxan (3 ml, 12.00 mmol) and the resulting mixture was stirred at RT for 2 hr 45 mins. The solvent was removed in vacuo to afford the title product as a hydrochloride salt; ¹H NMR (400 MHz, d6-DMSO) δ 10.79 (1H, s), 7.10 (1H, m), 6.98 (3H, m), 3.58 (2H, d), 2.09 (1H, m), 0.87 (6H, d).

### Intermediate RP

### 3,3,5-Trimethylindolin-2-one

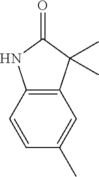

### Step 1: N-(2-bromo-4-methylphenyl)methacrylamide

2-Bromopyridin-3-amine (2.00 g, 11.56 mmol) in DCM (50 ml) and triethylamine (1.75 g, 17.34 mmol) was treated dropwise with methacryloyl chloride (1.33 g, 12.72 mmol) and stirred at RT for 1 hour. The mixture was partitioned between DCM and water. The organic phase was washed with water, brine, dried over MgSO₄, filtered and the solvent was removed in vacuo. The product was purified by chromatography on silica eluting with iso-hexane/EtOAc. The resulting residue was dissolved in MeOH and loaded onto a 10 g SCX cartridge, eluting with MeOH to afford the title compound;

¹H NMR (400 MHz, d6-DMSO) δ 9.35 (1H, s), 7.52 (1H, d), 7.39 (1H, d), 7.20 (1H, d of d), 5.90 (1H, s), 5.51 (1H, s), 2.30 (3H, s), 1.95 (3H, s).

### Step 2: N-(2-Bromo-4-methylphenyl)-N-((2-(trimethylsilyl)ethoxy)methyl)methacrylamide

N-(2-Bromo-4-methylphenyl)methacrylamide (step 1) (1.13 g, 4.45 mmol) in THF (50 ml) was treated with NaH (60% in oil) (0.233 g, 5.78 mmol) and stirred at RT for 10 minutes. SEM-Cl (0.89 g, 5.4 mmol) was added and the mixture was heated at reflux for 1 hour. After cooling to RT, the solvent was removed in vacuo and the residue was partitioned between DCM and water. The organic phase was washed with water, brine, dried over MgSO₄, filtered and the solvent was removed in vacuo. Purification by chromatography on silica eluting with iso-hexane/EtOAc afforded the title compound. ¹H NMR (400 MHz, d6-DMSO) δ 7.59 (1H, s), 7.28 (2H, m), 5.41 (1H, br), 5.05 (1H, br), 4.90 (1H, br), 4.60 (1H, br), 3.61 (2H, br), 2.32 (3H, s), 1.80 (3H, br), 0.89 (2H, m), 0.00 (9H, s).

### Step 3: 3,3,5-Trimethyl-1-((2-(trimethylsilyl)ethoxy)methyl)indolin-2-one

N-(2-Bromo-4-methylphenyl)-N-((2-(trimethylsilyl)ethoxy)methyl)methacrylamide (step 2) (610 mg, 1.59 mmol) in toluene (20 ml) was treated with tributyltin hydride (508 mg, 1.75 mmol) followed by 1,1′-azobis(cyclohexanecarbonitrile) (19.4 mg, 0.08 mmol). The resulting mixture was heated at reflux for 2 hours. After cooling to RT, the solvent was removed in vacuo and the residue was partitioned between DCM and water. The organic phase was washed with water, brine, dried over MgSO₄, filtered and the solvent was removed in vacuo. Purification by chromatography on silica eluting with iso-hexane/EtOAc afforded the title compound. ¹H NMR (400 MHz, d6-DMSO) δ 7.28 (1H, d), 7.13 (1H, m), 7.02 (1H, m), 5.17 (2H, s), 3.57 (2H, t), 2.39 (3H, s), 1.37 (6H, s), 0.91 (2H, t), 0.00 (9H, s).

### Step 4: 3,3,5-Trimethylindolin-2-one

A mixture comprising 3,3,5-trimethyl-1-((2-(trimethylsilyl)ethoxy)methyl)indolin-2-one (step 3) (360 mg, 1.18 mmol) and tetrabutylammonium fluoride (1M in THF) (2.36 ml, 2.36 mmol) was heated using microwave radiation at 120° C. for 1 hour followed by 140° C. for 1 hour. After cooling to RT, the solvent was removed in vacuo and the residue was partitioned between DCM and water. The organic phase was washed with water, brine, dried over MgSO₄, filtered and the solvent was removed in vacuo. Purification was carried out by chromatography on silica eluting with iso-hexane/EtOAc. The appropriate fractions were combined and concentrated in vacuo. The product crystallized and was triturated with iso-hexane to afford the title compound; LC-MS Rt 2.32 mins; MS m/z 176.12 [M+H]+; Method LowpH_v002. ¹HNMR (400 MHz, d6-DMSO) δ 10.20 (1H, s), 7.10 (1H, d), 6.97 (1H, d), 6.71 (1H, d), 2.25 (3H, s), 1.25 (6H, s).

### Intermediate RQ

### 1-(4-Amino-cyclohexylmethyl)-3,3-dimethyl-1,3-dihydro-indol-2-one

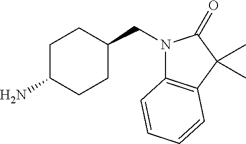

### Step 1: Methyl trans-4-(tert-butoxycarbonylamino)cyclohexanecarboxylate

Methyl trans-4-aminocyclohexanecarboxylate (43 g, 222 mmol) was added to MeOH (500 ml) to give a colourless solution. The solution was cooled to 10° C. and triethylamine (46.4 ml, 333 mmol) was added dropwise, followed by a solution of di-tert-butyldicarbonate (53.3 g, 244 mmol) in MeOH (400 ml) over 20 minutes. The reaction was allowed to warm to RT and stirred overnight. The mixture was evaporated to dryness under reduced pressure. The resulting colourless solid was dissolved in EtOAc (1000 ml) and the solution obtained was washed successively with 10% citric acid solution (100 ml), saturated sodium bicarbonate solution (2×100 ml) and saturated brine (100 ml), dried (MgSO₄) and evaporated under reduced pressure to give a colourless solid.

### Step 2: Trans-tert-butyl 4-(hydroxymethyl)cyclohexylcarbamate

Methyl trans-4-(tert-butoxycarbonylamino)cyclohexanecarboxylate (55.5 g, 216 mmol) was suspended in ethanol (900 ml) and THF (100 ml) and the mixture was cooled to 5° C. Granular calcium chloride (47.9 g, 43 μmol) was added portionwise to give a milky suspension. Sodium borohydride (32.6 g, 863 mmol) was added portionwise over 25 mins at 5° C. The reaction mixture (white emulsion) was stirred at 5° C. for 1 hour, the water bath was removed and then the reaction mixture was allowed to warm to room temperature and stirred at room temperature overnight. The reaction mixture was cooled to 10° C. and 5% potassium carbonate (200 ml) was added dropwise until the pH of the solution was pH11. A colourless precipitate formed which was filtered off. The solid was stirred with ethyl acetate (2000 ml) and water (500 ml). The organic layer was separated and washed with 0.5M HCl (200 ml), then washed with water (2×200 ml) and saturated brine (100 ml). The organic solution was dried over anhydrous MgSO₄, filtered and evaporated to give a white solid. The solid was dried under high vacuum overnight to constant weight; [M+H]+ 230.

### Step 3: Trans-trifluoro-methanesulfonic acid 4-tert-butoxycarbonylamino-cyclohexylmethyl ester

Trans-tert-butyl 4-(hydroxymethyl)cyclohexylcarbamate (step 1) (1.00 g, 4.36 mmol) was placed in a flask with DCM (50 ml) and pyridine (0.41 g, 5.23 mmol). The reaction mixture was cooled to 0° C. and then triflic anhydride (1.35 g, 4.80 mmol) was added dropwise. The reaction mixture was stirred at 0° C. for 1 hour and then partitioned between DCM and sat. ammonium chloride. The organic phase was dried over MgSO4, filtered and the solvent was removed in vacuo on an ice-cold water bath to give a beige solid. The product was purified by chromatography on silica eluting with iso-hexane/EtOAc to afford the title compound; ¹H NMR (d6-DMSO, 400 MHz) δ 6.72 (1H, d), 4.09 (2H, d), 3.03 (1H, m), 1.80 (2H, m), 1.70 (2H, m), 1.59 (1H, m), 1.38 (9H, s), 1.12 (2H, m), 1.01 (2H, m).

### Step 4: [4-(3,3-Dimethyl-2-oxo-2,3-dihydro-indol-1-ylmethyl)-cyclohexyl]-carbamic acid tert-butyl ester

3,3-Dimethyl-1,3-dihydro-indol-2-one (commercial) (268 mg, 1.66 mmol) in DMF (10 mL) was treated with NaH (60% in oil) (80 mg, 1.99 mmol) and the mixture was stirred at RT for 10 minutes. Trans-trifluoro-methanesulfonic acid 4-tert-butoxycarbonylamino-cyclohexylmethyl ester (600 mg, 1.66 mmol) was added and the reaction mixture was heated at 80° C. for 4 hours. The solvent was removed in vacuo and the residue was partitioned between DCM and water. The organic portion was passed through a phase separator and the solvent was removed in vacuo. Purification by chromatography on silica eluting with iso-hexane/EtOAc afforded the title compound. ¹H NMR (400 MHz, d6-DMSO) δ; 7.32 (1H, m), 7.22 (1H, m), 7.03 (1H, m), 3.49 (2H, d), 3.12 (1H, m), 1.73 (2H, m), 1.60 (3H, m), 1.37 (9H, s), 1.23 (6H, s), 1.02 (4H, m).

### Step 5: 1-(4-Amino-cyclohexylmethyl)-3,3-dimethyl-1,3-dihydro-indol-2-one

[4-(3,3-Dimethyl-2-oxo-2,3-dihydro-indol-1-ylmethyl)-cyclohexyl]-carbamic acid tert-butyl ester (step 4) in MeOH (2 mL) was treated with 4M HCl/dioxan (2 ml) and the resulting mixture was stirred at RT for 2 hours. The solvent was removed in vacuo and the residue was dissolved in MeOH and loaded onto a 10 g SCX cartridge. Eluting with MeOH followed by 2M ammonia/MeOH afforded the title product; LC-MS Rt 1.98 mins; MS m/z 273.3 [M+H]+; Method LowpH_v002. ¹H NMR (400 MHz, d6-DMSO) δ 7.32 (1H, d), 7.22 (1H, t), 7.03 (2H, m), 3.50 (2H, d), 3.31 (1H, m), 1.72 (2H, m), 1.67 (1H, m), 1.59 (2H, m), 1.27 (6H, s), 1.01 (2H, m), 0.91 (2H, m).

### Intermediate RR

### 1-(4-Amino-cyclohexylmethyl)-3-methyl-1,3-dihydro-benzoimidazol-2-one hydrochloride

This compound was prepared analogously to Intermediate RQ by replacing 3,3-dimethyl-1,3-dihydro-indol-2-one (commercial) (step 4) with 1-methyl-1H-benzo[d]imidazol-2(3H)-one (commercial).

LC-MS Rt 1.81 mins; MS m/z 260.23 [M+H]+; Method LowpH_v002.

¹H NMR (400 MHz, d6-DMSO) δ 7.92 (3H, br), 7.20 (1H, m), 7.13 (1H, m), 7.07 (2H, m), 3.69 (2H, d), 3.32 (3H, s), 2.92 (1H, m), 1.92 (2H, m), 1.73 (1H, m), 1.68 (2H, m), 1.23 (2H, m), 1.12 (2H, m).

### Intermediate RS

### 7-Methoxy-3,5-dimethyl-1,3-dihydro-imidazo[4,5-b]pyridin-2-one

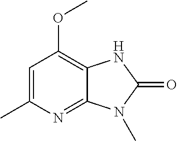

### Step 1: (4-Chloro-6-methyl-3-nitro-pyridin-2-yl)-methyl-amine

2,4-Dichloro-6-methyl-3-nitro-pyridine (1.00 g, 4.83 mmol) in methylamine (2M in THF) (9.66 ml, 19.3 mmol) under ice cooling and the reaction mixture was stirred at RT for 1 hour (exothermic reaction). The solvent was removed in vacuo and the residue was dissolved in EtOAc. The resulting ppt was filtered off and the solvent was removed in vacuo. Purification by chromatography on silica eluting with iso-hexane/EtOAc afforded the title product which was 60% pure. The compound was used in the next step without further purification.

### Step 2: (4-Methoxy-6-methyl-3-nitro-pyridin-2-yl)-methyl-amine

(4-Chloro-6-methyl-3-nitro-pyridin-2-yl)-methyl-amine (60% pure) (700 mg, 0.28 mmol) was dissolved in MeOH (10 ml). Sodium methoxide (25% in MeOH) (1.35 g, 6.25 mmol) was added and the reaction mixture was heated using microwave radiation at 100° C. for 1 hour. The resulting precipitate was filtered and purification by chromatography on silica eluting with iso-hexane/EtOAc afforded the title product;

¹H NMR (400 MHz, DMSO) δ 7.62 (1H, br), 6.40 (1H, s), 3.88 (3H, s), 2.85 (3H, d), 2.30 (3H, s).

### Step 3 and 4: 7-Methoxy-3,5-dimethyl-1,3-dihydro-imidazo[4,5-b]pyridin-2-one

The title compound was prepared analogously to Intermediate RA by replacing N-ethyl-2-nitroaniline with (4-methoxy-6-methyl-3-nitro-pyridin-2-yl)-methyl-amine (step 2). ¹H NMR (400 MHz, DMSO) δ 6.07 (1H, s), 5.29 (1H, m), 3.79 (3H, s), 3.72 (2H, s), 2.72 (3H, d), 2.20 (3H, s).

### Intermediate RT

### 2-Chloro-9-methyl-7,9-dihydro-purin-8-one

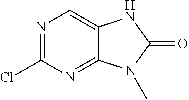

This compound was prepared analogously to 1-ethyl-1,3-dihydro-benzoimidazol-2-one (Intermediate RA) by replacing N-ethyl-benzene-1,2-diamine (Int RA, step 2) with 2-chloro-N4-methylpyrimidine-4,5-diamine. NMR (400 MHz, DMSO) δ 1.59 (1H, broad), 8.11 (1H, s), 3.28 (3H, s).

### Intermediate RU

### 2-Methoxy-9-methyl-7,9-dihydro-purin-8-one

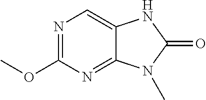

The title compound was prepared analogously to (4-methoxy-6-methyl-3-nitro-pyridin-2-yl)-methyl-amine (Intermediate RS, step 2) by replacing (4-chloro-6-methyl-3-nitro-pyridin-2-yl)-methyl-amine with 2-chloro-9-methyl-7,9-dihydro-purin-8-one (Int. RT); ¹H NMR (400 MHz, DMSO) δ 11.11 (1H, broad), 7.95 (1H, s), 3.85 (3H, s), 3.21 (3H, s).

### Intermediate RV

### 3,3-Dimethyl-1,3-dihydro-pyrrolo[3,2-b]pyridin-2-one

The title compound was prepared analogously to 3,3,5-trimethylindolin-2-one (Int. RP) by replacing 2-bromopyridin-3-amine (step 1) with 2-bromopyridin-3-amine. ¹H NMR (400 MHz, d6-DMSO) δ 9.57 (1H, s), 8.26 (1H, m), 7.96 (1H, m), 7.49 (1H, m), 5.95 (1H, s), 5.60 (1H, s), 1.98 (3H, s).

### Biological Data:

TABLE 1

| | CRF-1 IC50 |
|---|---|
| Example | (micromolar) |
| 1.1 | 0.065 |
| 2.1 | 0.149 |
| 2.10 | 0.201 |
| 2.3 | 0.242 |
| 2.4 | 0.900 |
| 2.42 | 0.275 |
| 2.43 | 0.416 |
| 2.5 | 0.009 |
| 2.52 | 0.612 |
| 2.7 | 0.166 |
| 2.8 | 0.347 |
| 2.9 | 0.016 |
| 3.1 | 0.205 |
| 3.3 | 0.028 |
| 3.6 | 0.145 |
| 3.7 | 0.091 |
| 3.8 | 0.037 |
| 4.2 | 0.118 |
| 5.2 | 0.126 |

TABLE 2

| | CRF-1 IC50 | CRF-2 IC50 |
|---|---|---|
| Example | (micromolar) | (micromolar) |
| 1.3 | 0.048 | 3.795 |
| 2.6 | 0.012 | 0.811 |
| 2.15 | 0.017 | 0.936 |
| 2.18 | 0.050 | 1.685 |
| 2.20 | 0.062 | 4.587 |
| 2.23 | 0.057 | 2.137 |
| 2.26 | 0.157 | 3.124 |
| 2.27 | 0.157 | 4.810 |
| 2.28 | 0.038 | 3.045 |
| 2.29 | 0.014 | 4.620 |
| 2.33 | 0.044 | 0.876 |
| 2.46 | 0.044 | 0.256 |
| 2.47 | 0.018 | 0.296 |
| 2.50 | 0.086 | 3.560 |
| 2.54 | 0.059 | 4.760 |
| 2.55 | 0.060 | 4.529 |
| 3.2 | 0.047 | 3.874 |
| 3.4 | 0.018 | 1.948 |

1. A compound of formula I; 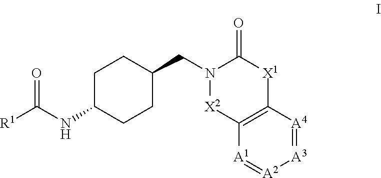
in which R¹ is phenyl or a 6-membered heteroaryl each of which may be optionally substituted by one or more substituents selected from the group alkyl C1 to 10, alkoxy C1 to 10, halogen and haloalkyl C1 to 10;
X¹ is a bond or is —CR²R³—, —NR⁴—, —O— or —CR⁵R⁶CR⁷R⁸—;
X² is a bond or is —CR⁹R¹⁰— or —CR¹¹R¹²CR¹³R¹⁴—;
provided that when X¹ is —CR⁵R⁶CR⁷R⁸— then X² is not —CR¹¹R¹²CR¹³R¹⁴— and only one of X¹ and X² may be a bond;
A¹ is —N— or CR¹⁵;
A² is CR¹⁶;
A³ is —N— or CR¹⁷;
A⁴ is —N— or CR¹⁸, provided that no more than two of A¹, A³ and A⁴ is —N—; or
R², R³, R⁵, R⁶, R⁷, R⁸, R⁹, R¹⁰, R¹¹, R¹², R¹³ and R¹⁴, which may be the same or different, are each hydrogen, alkyl C1 to 10 or halogen, or a pair of R² and R³, R⁵ and R⁶, R⁷ and R⁸, R⁹ and R¹⁰, R¹¹ and R¹², and R¹³ and R¹⁴, together form a 3- to 6-membered saturated carbocyclic or heterocyclic ring containing 1 or 2 heteroatoms
R⁴ is hydrogen or alkyl C1 to 10;
R¹⁵, R¹⁶, R¹⁷ and R¹⁸, which may be the same or different, are each hydrogen, alkyl C1 to 10, alkoxy C1 to 10, halogen or haloalkoxy C1 to 10;
and isomers thereof;
in free form or in salt form.

2. A compound according to claim 1 wherein the compound is of formula II; 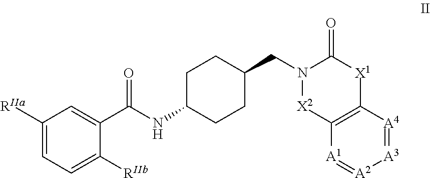
in which R^{IIa} and R^{IIb}, which may be the same or different, are each alkyl C1 to 10, halo or haloalkyl C1 to 10;
X¹, X², A¹, A², A³ and A⁴ are each as defined in claim 1;
and isomers thereof;
in free form or in salt form.

3. A compound according to claim 1 wherein the compound is of formula III; 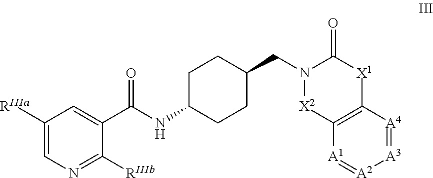
in which R^{IIIa} and R^{IIIb}, which may be the same or different, are each alkyl C1 to 10, halo or haloalkyl C1 to 10;
X¹, X², A¹, A², A³ and A⁴ are each as defined in claim 1;
and isomers thereof;
in free form or in salt form.

4. A compound according to claim 1 wherein the compound is of formula IV; 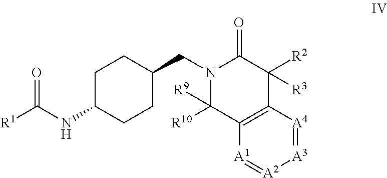
in which R¹, R², R³, R⁹, R¹⁰, A¹, A², A³ and A⁴ are each as defined in claim 1;
and isomers thereof;
in free form or in salt form.

5. A compound according to claim 1 wherein the compound is of formula V; 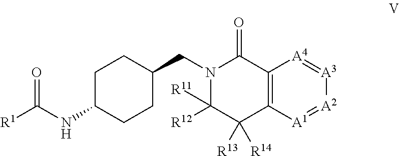
in which R¹, R¹¹, R¹², R¹³, R¹⁴, A¹, A², A³ and A⁴ are each as defined in claim 1;
and isomers thereof;
in free form or in salt form.

6. A compound according to claim 1 wherein the compound is of formula VI; 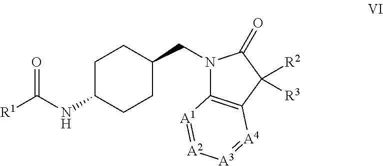
in which R¹, R², R³, A¹, A², A³ and A⁴ are each as defined in claim 1;
and isomers thereof;
in free form or in salt form.

7. A compound according to claim 1 which is selected from the group consisting of:
trans-2-chloro-N-[4-(6-chloro-2-oxo-2,3-dihydro-indol-1-ylmethyl)-cyclohexyl]-5-trifluoromethyl-benzamide;
trans-2-chloro-N-[4-(2-oxo-2,3-dihydro-indol-1-ylmethyl)-cyclohexyl]-5-trifluoromethyl-benzamide;
trans-5-chloro-N-[4-(3,3-dimethyl-2-oxo-2,3-dihydro-indol-1-ylmethyl)-cyclohexyl]-2-methyl-icotinamide;
trans-2-chloro-N-[4-(6-chloro-3,3-difluoro-2-oxo-2,3-dihydro-indol-1-ylmethyl)-cyclohexyl]-5-trifluoromethyl-benzamide;
trans-2-chloro-N-[4-(3,3-dimethyl-2-oxo-2,3-dihydro-pyrrolo[2,3-b]pyridin-1-ylmethyl)-cyclohexyl]-5-trifluoromethyl-benzamide;
trans-5-chloro-N-[4-(3,3-dimethyl-2-oxo-2,3-dihydro-pyrrolo[2,3-b]pyridin-1-ylmethyl)-cyclohexyl]-2-methyl-nicotinamide;
trans-2-chloro-N-[4-(2-oxo-2,3-dihydro-pyrrolo[2,3-b]pyridin-1-ylmethyl)-cyclohexyl]-5-trifluoromethyl-benzamide;
trans-2-chloro-N-{-4-((5′-fluoro-2′-oxospiro[cyclopropane-1,3′-indoline]-1′-yl)methyl)cyclohexyl}-5-(trifluoromethyl)benzamide;
trans-5-chloro-N-[-4-((5′-fluoro-2′-oxospiro[cyclopropane-1,3′-indoline]-1′-yl)methyl)cyclohexyl]-2-methyl nicotinamide;
trans-2-chloro-N-[4-(5-methoxy-3,3-dimethyl-2-oxo-2,3-dihydro-indol-1-ylmethyl)-cyclohexyl]-5-trifluoromethyl-benzamide;
trans-2-chloro-N-[4-(6-methoxy-3,3-dimethyl-2-oxo-2,3-dihydro-pyrrolo[3,2-c]pyridin-1-ylmethyl)-cyclohexyl]-5-trifluoromethyl-benzamide;
trans-2-chloro-N-[4-((R)-3-fluoro-3-methyl-2-oxo-2,3-dihydro-indol-1-ylmethyl)-cyclohexyl]-5-trifluoromethyl-benzamide;
trans-2-chloro-N-[4-((S)-3-fluoro-3-methyl-2-oxo-2,3-dihydro-indol-1-ylmethyl)-cyclohexyl]-5-trifluoro methyl-benzamide;
trans-2-chloro-N-[4-(3,3-dimethyl-2-oxo-2,3-dihydro-indol-1-ylmethyl)-cyclohexyl]-5-trifluoromethyl-benzamide;
trans-2-chloro-N-[4-(3,3-dimethyl-2-oxo-5-trifluoromethoxy-2,3-dihydro-indol-1-ylmethyl)-cyclohexyl]-5-trifluoromethyl-benzamide;
trans-5-chloro-N-[4-(5-fluoro-3,3-dimethyl-2-oxo-2,3-dihydro-indol-1-ylmethyl)-cyclohexyl]-2-methyl-nicotinamide;
trans-5-chloro-N-[4-(4-methoxy-3,3-dimethyl-2-oxo-2,3-dihydro-indol-1-ylmethyl)-cyclohexyl]-2-methyl-nicotinamide;
trans-5-chloro-N-[4-(7-chloro-3,3-dimethyl-2-oxo-2,3-dihydro-indol-1-ylmethyl)-cyclohexyl]-2-methyl-nicotinamide;
trans-5-chloro-N-[4-(3,3-difluoro-2-oxo-2,3-dihydro-indol-1-ylmethyl)-cyclohexyl]-2-methyl-nicotinamide;
trans-2-chloro-N-[4-(3,3-difluoro-2-oxo-2,3-dihydro-indol-1-ylmethyl)-cyclohexyl]-5-trifluoromethyl-benzamide;
trans-2-chloro-N-[4-(7-methoxy-3,3-dimethyl-2-oxo-2,3-dihydro-indol-1-ylmethyl)-cyclohexyl]-5-trifluoromethyl-benzamide;
trans-5-chloro-N-[4-((R)-3-fluoro-3-methyl-2-oxo-2,3-dihydro-indol-1-ylmethyl)-cyclohexyl]-2-methyl-nicotinamide;
trans-5-chloro-N-[4-((S)-3-fluoro-3-methyl-2-oxo-2,3-dihydro-indol-1-ylmethyl)-cyclohexyl]-2-methyl-nicotinamide;
trans-5-chloro-2-methyl-N-(-4-((2-oxospiro[indoline-3,4′-piperidine]-1-yl)methyl)cyclohexyl)nicotinamide;
trans-2-chloro-N-[4-(6-methoxy-3,3-dimethyl-2-oxo-2,3-dihydro-indol-1-ylmethyl)-cyclohexyl]-5-trifluoromethyl-benzamide;
trans-2-chloro-5-trifluoromethyl-N-[4-(3,3,7-trimethyl-2-oxo-2,3-dihydro-indol-1-ylmethyl)-cyclohexyl]-benzamide;
trans-2-chloro-5-trifluoromethyl-N-[4-(3,3,4-trimethyl-2-oxo-2,3-dihydro-indol-1-ylmethyl)-cyclohexyl]-benzamide;
trans-5-chloro-N-[4-(4-chloro-3,3-dimethyl-2-oxo-2,3-dihydro-indol-1-ylmethyl)-cyclohexyl]-2-methyl-nicotinamide;
trans-5-chloro-N-[4-(6-chloro-3,3-difluoro-2-oxo-2,3-dihydro-indol-1-ylmethyl)-cyclohexyl]-2-methyl-nicotinamide;
trans-5-chloro-2-methyl-N-[4-(3,3,4-trimethyl-2-oxo-2,3-dihydro-indol-1-ylmethyl)-cyclohexyl]-nicotinamide;
trans-5-chloro-N-[4-(6-methoxy-3,3-dimethyl-2-oxo-2,3-dihydro-indol-1-ylmethyl)-cyclohexyl]-2-methyl-nicotinamide;
trans-5-chloro-N-[4-(6-chloro-3,3-dimethyl-2-oxo-2,3-dihydro-indol-1-ylmethyl)-cyclohexyl]-2-methyl-nicotinamide;
trans-5-chloro-N-[4-(6-methoxy-3,3-dimethyl-2-oxo-2,3-dihydro-pyrrolo[3,2-c]pyridin-1-ylmethyl)-cyclohexyl]-2-methyl-nicotinamide;
trans-5-chloro-N-[4-(-3-fluoro-3,5-dimethyl-2-oxo-2,3-dihydro-indol-1-ylmethyl)-cyclohexyl]-2-methyl-nicotinamide;
trans-5-chloro-N-[4-(-3-fluoro-3,5-dimethyl-2-oxo-2,3-dihydro-indol-1-ylmethyl)-cyclohexyl]-2-methyl-nicotinamide;
trans-5-chloro-N-[4-(-6-chloro-3-fluoro-3-methyl-2-oxo-2,3-dihydro-indol-1-ylmethyl)-cyclohexyl]-2-methyl-nicotinamide;
trans-5-chloro-N-[4-(-6-chloro-3-fluoro-3-methyl-2-oxo-2,3-dihydro-indol-1-ylmethyl)-cyclohexyl]-2-methyl-nicotinamide;
trans-2-chloro-N-[4-(5-methoxy-1-oxo-3,4-dihydro-1H-isoquinolin-2-ylmethyl)-cyclohexyl]-5-trifluoromethyl-benzamide;
trans-2-chloro-N-[4-(3-oxo-3,4-dihydro-1H-isoquinolin-2-ylmethyl)-cyclohexyl]-5-trifluoromethyl-benzamide;
trans-5-chloro-2-methyl-N-[4-(-3,5,6-trifluoro-3-methyl-2-oxo-2,3-dihydro-indol-1-ylmethyl)-cyclohexyl]-nicotinamide;
trans-5-chloro-2-methyl-N-[4-(-3,5,6-trifluoro-3-methyl-2-oxo-2,3-dihydro-indol-1-ylmethyl)-cyclohexyl]-nicotinamide;
trans-5-chloro-2-methyl-N-[4-(2-oxo-oxazolo[4,5-b]pyridin-3-ylmethyl)-cyclohexyl]-nicotinamide;
trans-5-chloro-2-methyl-N-[4-(2-oxo-benzooxazol-3-ylmethyl)-cyclohexyl]-nicotinamide;
trans-5-chloro-N-[4-(3,6-dimethyl-2-oxo-2,3-dihydro-imidazo[4,5-b]pyridin-1-ylmethyl)-cyclohexyl]-2-methyl-nicotinamide;
trans-5-chloro-N-[4-(3-ethyl-2-oxo-2,3-dihydro-imidazo[4,5-c]pyridin-1-ylmethyl)-cyclohexyl]-2-methyl-nicotinamide;
trans-5-chloro-N-[4-(3,7-dimethyl-2-oxo-2,3-dihydro-imidazo[4,5-b]pyridin-1-ylmethyl)-cyclohexyl]-2-methyl-nicotinamide;
trans-5-chloro-N-[4-(3,5-dimethyl-2-oxo-2,3-dihydro-imidazo[4,5-b]pyridin-1-ylmethyl)-cyclohexyl]-2-methyl-nicotinamide;
trans-5-chloro-N-[4-(1,5-dimethyl-2-oxo-1,2-dihydro-imidazo[4,5-b]pyridin-3-ylmethyl)-cyclohexyl]-2-methyl-nicotinamide;
trans-5-chloro-N-[4-(3-ethyl-2-oxo-2,3-dihydro-benzoimidazol-1-ylmethyl)-cyclohexyl]-2-methyl-nicotinamide;
trans-5-chloro-N-[4-(3-isobutyl-2-oxo-2,3-dihydro-benzoimidazol-1-ylmethyl)-cyclohexyl]-2-methyl-nicotinamide;
trans-5-chloro-N-[4-(5-methoxy-3-methyl-2-oxo-2,3-dihydro-imidazo[4,5-b]pyridin-1-ylmethyl)-cyclohexyl]-2-methyl-nicotinamide;
trans-5-chloro-2-methyl-N-[4-(3,3,5-trimethyl-2-oxo-2,3-dihydro-indol-1-ylmethyl)-cyclohexyl]-nicotinamide;
trans-5-chloro-2-methyl-N-[4-(3-methyl-2-oxo-2,3-dihydro-benzoimidazol-1-ylmethyl)-cyclohexyl]-nicotinamide;
trans-5-chloro-2-methyl-N-[4-(1-methyl-2-oxo-1,2-dihydro-imidazo[4,5-b]pyridin-3-ylmethyl)-cyclohexyl]-nicotinamide;
trans-N-[4-(3,3-Dimethyl-2-oxo-2,3-dihydro-indol-1-ylmethyl)-cyclohexyl]-2-methyl-5-trifluoromethyl-nicotinamide;
trans-5-chloro-2-methyl-N-[4-(2-oxo-2,3-dihydro-benzoimidazol-1-ylmethyl)-cyclohexyl]-nicotinamide;
Enantiomer 1 of trans-5-chloro-N-[4-(3-fluoro-3,5,6-trimethyl-2-oxo-2,3-dihydro-indol-1-ylmethyl)-cyclohexyl]-2-methyl-nicotinamide;
Enantiomer 2 of trans-5-chloro-N-[4-(3-fluoro-3,5,6-trimethyl-2-oxo-2,3-dihydro-indol-1-ylmethyl)-cyclohexyl]-2-methyl-nicotinamide;
trans-5-chloro-N-[4-(7-methoxy-3,5-dimethyl-2-oxo-2,3-dihydro-imidazo[4,5-b]pyridin-1-ylmethyl)-cyclohexyl]-2-methyl-nicotinamide;
trans-5-chloro-N-[4-(3,3-dimethyl-2-oxo-2,3-dihydro-pyrrolo[3,2-b]pyridin-1-ylmethyl)-cyclohexyl]-2-methyl-nicotinamide;
trans-5-chloro-N-[4-(2-methoxy-9-methyl-8-oxo-8,9-dihydro-purin-7-ylmethyl)-cyclohexyl]-2-methyl-nicotinamide;
trans-5-chloro-N-[4-(2-chloro-9-methyl-8-oxo-8,9-dihydro-purin-7-ylmethyl)-cyclohexyl]-2-methyl-nicotinamide;
trans-2-chloro-N-[4-(5-chloro-2-oxo-2,3-dihydro-indol-1-ylmethyl)-cyclohexyl]-5-trifluoromethyl-benzamide;
trans-2-chloro-N-[4-(6-fluoro-2-oxo-2,3-dihydro-indol-1-ylmethyl)-cyclohexyl]-5-trifluoromethyl-benzamide;
trans-2-chloro-N-[4-(5-fluoro-3,3-dimethyl-2-oxo-2,3-dihydro-indol-1-ylmethyl)-cyclohexyl]-5-trifluoromethyl-benzamide;
trans-2-chloro-N-[4-(3-ethyl-2-oxo-2,3-dihydro-benzoimidazol-1-ylmethyl)-cyclohexyl]-5-trifluoromethyl-benzamide;
trans-2-chloro-N-[4-(3-methyl-2-oxo-2,3-dihydro-imidazo[4,5-b]pyridin-1-ylmethyl)-cyclohexyl]-5-trifluoromethyl-benzamide;
trans-2-chloro-N-[4-(1-methyl-2-oxo-1,2-dihydro-imidazo[4,5-b]pyridin-3-ylmethyl)-cyclohexyl]-5-trifluoromethyl-benzamide;
trans-2-chloro-N-[4-(2-oxo-2,3-dihydro-benzoimidazol-1-ylmethyl)-cyclohexyl]-5-trifluoromethyl-benzamide;
trans-2-chloro-N-[4-(3-methyl-2-oxo-2,3-dihydro-benzoimidazol-1-ylmethyl)-cyclohexyl]-5-trifluoromethyl-benzamide;
trans-4-Fluoro-N-[4-(3-methyl-2-oxo-2,3-dihydro-benzoimidazol-1-ylmethyl)-cyclohexyl]-3-trifluoromethyl-benzamide trifluoroacetate;
trans-2,5-Dichloro-N-[4-(3-methyl-2-oxo-2,3-dihydro-benzoimidazol-1-ylmethyl)-cyclohexyl]-benzamide trifluoroacetate;
trans-N-[4-(3,3-Dimethyl-2-oxo-2,3-dihydro-indol-1-ylmethyl)-cyclohexyl]-4-fluoro-3-trifluoromethyl-benzamide;
trans-2,5-Dichloro-N-[4-(3,3-dimethyl-2-oxo-2,3-dihydro-indol-1-ylmethyl)-cyclohexyl]-benzamide; and
trans-N-[4-(3,3-Dimethyl-2-oxo-2,3-dihydro-indol-1-ylmethyl)-cyclohexyl]-3-methoxy-benzamide;
and isomers thereof;
in free or in salt form.

8. A method to treat a condition characterized by increased endogenous levels of CRF, which comprises administering to a subject in need thereof an effective amount of a compound according to claim 1 in free form or in pharmaceutically acceptable salt form.

9. The method of claim 8, wherein the condition is a gastrointestinal disorder, a depressive disorder, a mood disorder, a schizophrenic disorder, a neurodegenerative disorder, or pain.

10. The method of claim 8, wherein the condition is an appetite dysfunction, a sleep disorder, a cognitive disorder, a memory condition, substance dependence, inflammation, a fertility problem, an allergic disorder, emesis, or neurotoxic injury.

11. A method to treat a condition wherein the hypothalamic pituitary axis (HPA) is disregulated, which comprises administering to a subject in need thereof an effective amount of a compound according to claim 1 in free form or in pharmaceutically acceptable salt form.

12. A method to treat a condition characterized by a barrier dysfunction of mucous epithelia, epidermis or endothelia, which comprises administering an effective amount of a dual antagonist of corticotropin releasing factor receptor 1 (CRF-1) and corticotropin releasing factor receptor 2 (CRF-2).

13. A pharmaceutical composition comprising a compound of formula I according to claim 1 in free form or in pharmaceutically acceptable salt form, in association with a pharmaceutically acceptable adjuvant, diluent or carrier.

14. A pharmaceutical composition comprising a compound of formula I according to claim 1 in free form or in pharmaceutically acceptable salt form, in combination with another therapeutically active ingredient, optionally in association with a pharmaceutically acceptable adjuvant, diluent or carrier.

15. A compound of formula VII; 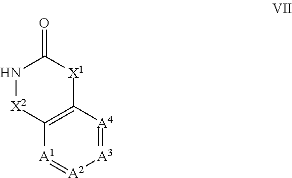
in which X¹, X², A¹, A², A³ and A⁴ are each as defined in claim 1;
and isomers thereof;
in free form or in salt form.
